(19) United States
(12) Patent Application Publication
(10) Pub. No.: US 2025/0391558 A1
(43) Pub. Date: Dec. 25, 2025

(54) HEALTH INFORMATION USER INTERFACES

(71) Applicant: Apple Inc., Cupertino, CA (US)

(72) Inventors: Nicholas D. FELTON, Sunnyvale, CA (US); Niharika BEDEKAR, San Francisco, CA (US); Margaret A. BIGNELL, San Francisco, CA (US); Asha N. CHESNUTT, Portland, OR (US); Jessica FICKLIN, Encinitas, CA (US); Michael PANGBURN, San Jose, CA (US); Nathan G. RACKLYEFT, Carlsbad, CA (US); Catherine S. THOMAS, San Diego, CA (US); Gary I. BUTCHER, Los Gatos, CA (US)

(21) Appl. No.: 19/224,076

(22) Filed: May 30, 2025

Related U.S. Application Data
(60) Provisional application No. 63/657,923, filed on Jun. 9, 2024.

(51) Int. Cl. G16H 40/67; G16H 50/30

(52) U.S. Cl. CPC G16H 40/67; G16H 50/30

(57) ABSTRACT

The present disclosure generally relates to tracking and presenting user health metrics.

### CROSS-REFERENCE TO RELATED APPLICATIONS

This application claims priority to U.S. Provisional Patent Application Ser. No. 63/657,923, entitled “HEALTH INFORMATION USER INTERFACES,” filed on Jun. 9, 2024, the contents of which are hereby incorporated by reference in their entirety.

### FIELD

The present disclosure relates generally to computer user interfaces, and more specifically to techniques for tracking and presenting user health metrics.

### BACKGROUND

Electronic devices can be used to track health metrics and display information pertaining to health metrics of a user.

### BRIEF SUMMARY

Some techniques for tracking and presenting user health metrics using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for tracking and presenting user health metrics. Such methods and interfaces optionally complement or replace other methods for tracking and presenting user health metrics. Such methods and interfaces reduce the cognitive burden on a user and produce a more efficient human-machine interface. For battery-operated computing devices, such methods and interfaces conserve power and increase the time between battery charges.

In accordance with some embodiments, a method is described. The method is performed at a computer system that is in communication with one or more display generation components and one or more input devices, and comprises: displaying, via the one or more display generation components, a daily metrics user interface, including displaying, within the daily metrics user interface, first vital metric information that is representative of a first day, wherein the first vital metric information includes two or more vital metrics; while displaying the daily metrics user interface, detecting, via the one or more input devices, a first user input; and in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a non-transitory computer-readable storage medium is described. The non-transitory computer-readable storage medium stores one or more programs configured to be executed by one or more processors of a computer system that is in communication with one or more display generation components and one or more input devices, and the one or more programs include instructions for: displaying, via the one or more display generation components, a daily metrics user interface, including displaying, within the daily metrics user interface, first vital metric information that is representative of a first day, wherein the first vital metric information includes two or more vital metrics; while displaying the daily metrics user interface, detecting, via the one or more input devices, a first user input; and in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a transitory computer-readable storage medium is described. The transitory computer-readable storage medium stores one or more programs configured to be executed by one or more processors of a computer system that is in communication with one or more display generation components and one or more input devices, and the one or more programs include instructions for: displaying, via the one or more display generation components, a daily metrics user interface, including displaying, within the daily metrics user interface, first vital metric information that is representative of a first day, wherein the first vital metric information includes two or more vital metrics; while displaying the daily metrics user interface, detecting, via the one or more input devices, a first user input; and in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a computer system is described. The computer system is configured to communicate with one or more display generation components and one or more input devices, and comprises: one or more processors; and memory storing one or more programs configured to be executed by the one or more processors, the one or more programs including instructions for: displaying, via the one or more display generation components, a daily metrics user interface, including displaying, within the daily metrics user interface, first vital metric information that is representative of a first day, wherein the first vital metric information includes two or more vital metrics; while displaying the daily metrics user interface, detecting, via the one or more input devices, a first user input; and in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a computer system is described. The computer system is configured to communicate with one or more display generation components and one or more input devices, and comprises: means for displaying, via the one or more display generation components, a daily metrics user interface, including displaying, within the daily metrics user interface, first vital metric information that is representative of a first day, wherein the first vital metric information includes two or more vital metrics; means for, while displaying the daily metrics user interface, detecting, via the one or more input devices, a first user input; and means for, in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a computer program product is described. The computer program product comprises one or more programs configured to be executed by one or more processors of a computer system that is in communication with one or more display generation components and one or more input devices, the one or more programs including instructions for: displaying, via the one or more display generation components, a daily metrics user interface, including displaying, within the daily metrics user interface, first vital metric information that is representative of a first day, wherein the first vital metric information includes two or more vital metrics; while displaying the daily metrics user interface, detecting, via the one or more input devices, a first user input; and in response to detecting the first user input: in accordance with a determination that a first vital metric of the two or more vital metrics was selected when the first user input was received, displaying, via the one or more display generation components, a first weekly metrics user interface, wherein the first weekly metrics user interface includes first weekly vital metric information that is representative of a first week and displays information pertaining to the first vital metric for the first week without displaying information corresponding to a second vital metric different from the first vital metric; and in accordance with a determination that the second vital metric different from the first vital metric was selected when the first user input was received, displaying, via the one or more display generation components, a second weekly metrics user interface, wherein the second weekly metrics user interface includes second weekly vital metric information that is representative of the first week and displays information pertaining to the second vital metric for the first week without displaying information corresponding to first vital metric.

In accordance with some embodiments, a method is described. The method is performed at a computer system that is in communication with one or more display generation components, and comprises: displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a non-transitory computer-readable storage medium is described. The non-transitory computer-readable storage medium stores one or more programs configured to be executed by one or more processors of a computer system that is in communication with one or more display generation components, the one or more programs including instructions for: displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a transitory computer-readable storage medium is described. The transitory computer-readable storage medium stores one or more programs configured to be executed by one or more processors of a computer system that is in communication with one or more display generation components, the one or more programs including instructions for: displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a computer system is described. The computer system is configured to communicate with one or more display generation components, and comprises: one or more processors; and memory storing one or more programs configured to be executed by the one or more processors, the one or more programs including instructions for: displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a computer system is described. The computer system is configured to communicate with one or more display generation components, and comprises: means for displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a computer program product is described. The computer program product comprises one or more programs configured to be executed by one or more processors of a computer system that is in communication with one or more display generation components, the one or more programs including instructions for: displaying, via the one or more display generation components, a metrics user interface that is representative of a first duration of time, wherein: the metrics user interface includes a first region that is representative of a first day of the first duration of time; and the first region includes a first set of objects that are representative of a set of vital metrics for the first day, wherein the first set of vital metrics includes two or more vital metrics; and displaying the metrics user interface includes: in accordance with a determination that at least one or more vital metrics of the set of vital metrics are within a threshold range for the first day, displaying a first subset of the first set of objects within a first section of the first region, wherein the first section of the first region is representative of the threshold range; and in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

In accordance with some embodiments, a method is described. The method is performed at a computer system that is in communication with one or more display generation components and one or more input devices, and comprises: receiving, via the one or more input devices, first vital metric information pertaining to a user, wherein the first vital metric information pertaining to the user corresponds to two or more vital metrics; determining, based on the first vital metric information, baseline value ranges for the two or more vital metrics, including a first baseline value range for a first vital metric of the two or more vital metrics and a second baseline value range for a second vital metric of the two or more vital metrics; subsequent to determining the baseline value ranges for the two or more vital metrics, receiving, via the one or more input devices, second vital metric information pertaining to the user; and in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a non-transitory computer-readable storage medium is described. The non-transitory computer-readable storage medium stores one or more programs configured to be executed by one or more processors of a computer system that is in communication with one or more display generation components and one or more input devices, the one or more programs including instructions for: receiving, via the one or more input devices, first vital metric information pertaining to a user, wherein the first vital metric information pertaining to the user corresponds to two or more vital metrics; determining, based on the first vital metric information, baseline value ranges for the two or more vital metrics, including a first baseline value range for a first vital metric of the two or more vital metrics and a second baseline value range for a second vital metric of the two or more vital metrics; subsequent to determining the baseline value ranges for the two or more vital metrics, receiving, via the one or more input devices, second vital metric information pertaining to the user; and in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a transitory computer-readable storage medium is described. The transitory computer-readable storage medium stores one or more programs configured to be executed by one or more processors of a computer system that is in communication with one or more display generation components and one or more input devices, the one or more programs including instructions for: receiving, via the one or more input devices, first vital metric information pertaining to a user, wherein the first vital metric information pertaining to the user corresponds to two or more vital metrics; determining, based on the first vital metric information, baseline value ranges for the two or more vital metrics, including a first baseline value range for a first vital metric of the two or more vital metrics and a second baseline value range for a second vital metric of the two or more vital metrics; subsequent to determining the baseline value ranges for the two or more vital metrics, receiving, via the one or more input devices, second vital metric information pertaining to the user; and in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a computer system is described. The computer system is configured to communicate with one or more display generation components and one or more input devices, and comprises: one or more processors; and memory storing one or more programs configured to be executed by the one or more processors, the one or more programs including instructions for: receiving, via the one or more input devices, first vital metric information pertaining to a user, wherein the first vital metric information pertaining to the user corresponds to two or more vital metrics; determining, based on the first vital metric information, baseline value ranges for the two or more vital metrics, including a first baseline value range for a first vital metric of the two or more vital metrics and a second baseline value range for a second vital metric of the two or more vital metrics; subsequent to determining the baseline value ranges for the two or more vital metrics, receiving, via the one or more input devices, second vital metric information pertaining to the user; and in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a computer system is described. The computer system is configured to communicate with one or more display generation components and one or more input devices, and comprises: means for receiving, via the one or more input devices, first vital metric information pertaining to a user, wherein the first vital metric information pertaining to the user corresponds to two or more vital metrics; means for determining, based on the first vital metric information, baseline value ranges for the two or more vital metrics, including a first baseline value range for a first vital metric of the two or more vital metrics and a second baseline value range for a second vital metric of the two or more vital metrics; means for, subsequent to determining the baseline value ranges for the two or more vital metrics, receiving, via the one or more input devices, second vital metric information pertaining to the user; and means for, in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a computer program product is described. The computer program product comprises one or more programs configured to be executed by one or more processors of a computer system that is in communication with one or more display generation components and one or more input devices, the one or more programs including instructions for: receiving, via the one or more input devices, first vital metric information pertaining to a user, wherein the first vital metric information pertaining to the user corresponds to two or more vital metrics; determining, based on the first vital metric information, baseline value ranges for the two or more vital metrics, including a first baseline value range for a first vital metric of the two or more vital metrics and a second baseline value range for a second vital metric of the two or more vital metrics; subsequent to determining the baseline value ranges for the two or more vital metrics, receiving, via the one or more input devices, second vital metric information pertaining to the user; and in response to receiving the second vital metric information pertaining to the user: in accordance with a determination that the second vital metric information is indicative of the first vital metric falling outside of the first baseline value range, displaying, via the one or more display generation components, a first alert that is indicative of the first vital metric falling outside of the first threshold range, wherein the first alert identifies a first potential reason for the first vital metric falling outside of the first threshold range.

In accordance with some embodiments, a method is described. The method is performed at a computer system that is in communication with one or more display generation components and one or more input devices, and comprises: detecting, via the one or more input devices, that a user has completed a first workout; subsequent to detecting that the user has completed the first workout, detecting, via the one or more input devices, one or more user inputs corresponding to user entry of a first workout effort value for a workout effort metric corresponding to the first workout; and after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

In accordance with some embodiments, a non-transitory computer-readable storage medium is described. The non-transitory computer-readable storage medium stores one or more programs configured to be executed by one or more processors of a computer system that is in communication with one or more display generation components and one or more input devices, the one or more programs including instructions for: detecting, via the one or more input devices, that a user has completed a first workout; subsequent to detecting that the user has completed the first workout, detecting, via the one or more input devices, one or more user inputs corresponding to user entry of a first workout effort value for a workout effort metric corresponding to the first workout; and after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

In accordance with some embodiments, a transitory computer-readable storage medium is described. The transitory computer-readable storage medium stores one or more programs configured to be executed by one or more processors of a computer system that is in communication with one or more display generation components and one or more input devices, the one or more programs including instructions for: detecting, via the one or more input devices, that a user has completed a first workout; subsequent to detecting that the user has completed the first workout, detecting, via the one or more input devices, one or more user inputs corresponding to user entry of a first workout effort value for a workout effort metric corresponding to the first workout; and after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

In accordance with some embodiments, a computer system is described. The computer system is configured to communicate with one or more display generation components and one or more input devices, and comprises: one or more processors; and memory storing one or more programs configured to be executed by the one or more processors, the one or more programs including instructions for: detecting, via the one or more input devices, that a user has completed a first workout; subsequent to detecting that the user has completed the first workout, detecting, via the one or more input devices, one or more user inputs corresponding to user entry of a first workout effort value for a workout effort metric corresponding to the first workout; and after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

In accordance with some embodiments, a computer system is described. The computer system is configured to communicate with one or more display generation components and one or more input devices, and comprises: means for detecting, via the one or more input devices, that a user has completed a first workout; means for, subsequent to detecting that the user has completed the first workout, detecting, via the one or more input devices, one or more user inputs corresponding to user entry of a first workout effort value for a workout effort metric corresponding to the first workout; and means for, after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

In accordance with some embodiments, a computer program product is described. The computer program product comprises one or more programs configured to be executed by one or more processors of a computer system that is in communication with one or more display generation components and one or more input devices, the one or more programs including instructions for: detecting, via the one or more input devices, that a user has completed a first workout; subsequent to detecting that the user has completed the first workout, detecting, via the one or more input devices, one or more user inputs corresponding to user entry of a first workout effort value for a workout effort metric corresponding to the first workout; and after detecting the one or more user inputs corresponding to user entry of the first workout effort value for the workout effort metric corresponding to the first workout, displaying, via the one or more display generation components, a workload user interface, wherein: the workload user interface displays a first workload indication that is representative of a first day and is indicative of a first workload value that is calculated for the user for the first day, wherein the first workload indication and the first workload value are determined based on the first workout effort value for the workout effort metric corresponding to the first workout that was entered by the user.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for tracking and presenting user health metrics, thereby increasing the effectiveness, efficiency, and user satisfaction with such devices. Such methods and interfaces may complement or replace other methods for tracking and presenting user health metrics.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3A is a block diagram of an exemplary multifunction device with a display and a touch-sensitive surface in accordance with some embodiments.

FIGS. 3B-3G illustrate the use of Application Programming Interfaces (APIs) to perform operations.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6AC illustrate exemplary user interfaces for tracking and providing user health metrics, in accordance with some embodiments.

FIG. 7 illustrates a flow diagram depicting a method for tracking and providing user health metrics, in accordance with some embodiments.

FIG. 8 illustrates a flow diagram depicting a method for tracking and providing user health metrics, in accordance with some embodiments.

FIG. 9 illustrates a flow diagram depicting a method for tracking and providing user health metrics, in accordance with some embodiments.

FIGS. 10A-10AG illustrate exemplary user interfaces for tracking and providing user health metrics, in accordance with some embodiments.

FIG. 11 illustrates a flow diagram depicting a method for tracking and providing user health metrics, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for tracking and presenting user health metrics. Such techniques can reduce the cognitive burden on a user who tracks and/or accesses health data, thereby enhancing productivity. Further, such techniques can reduce processor and battery power otherwise wasted on redundant user inputs.

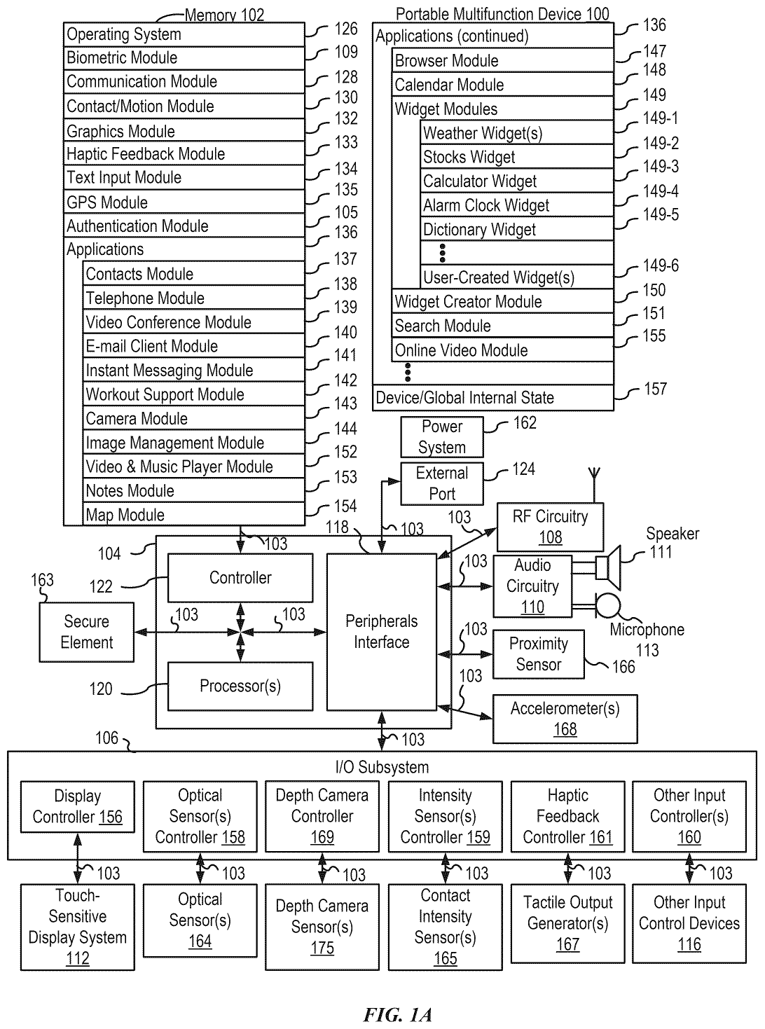

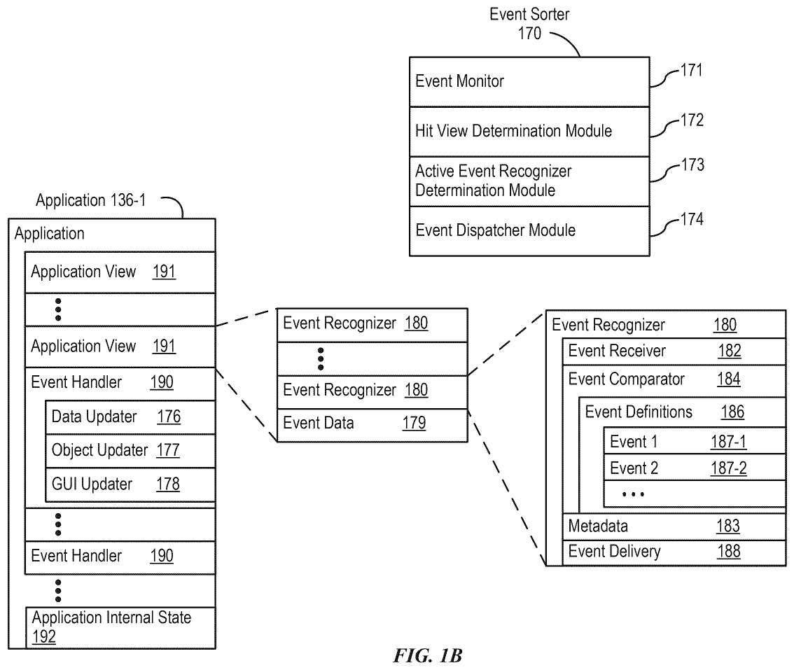

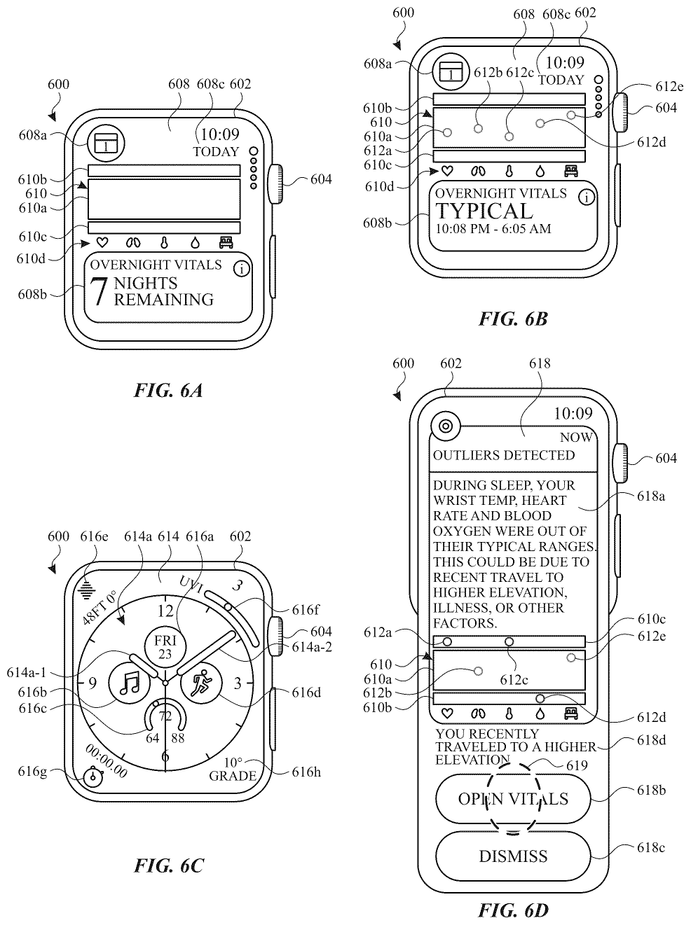

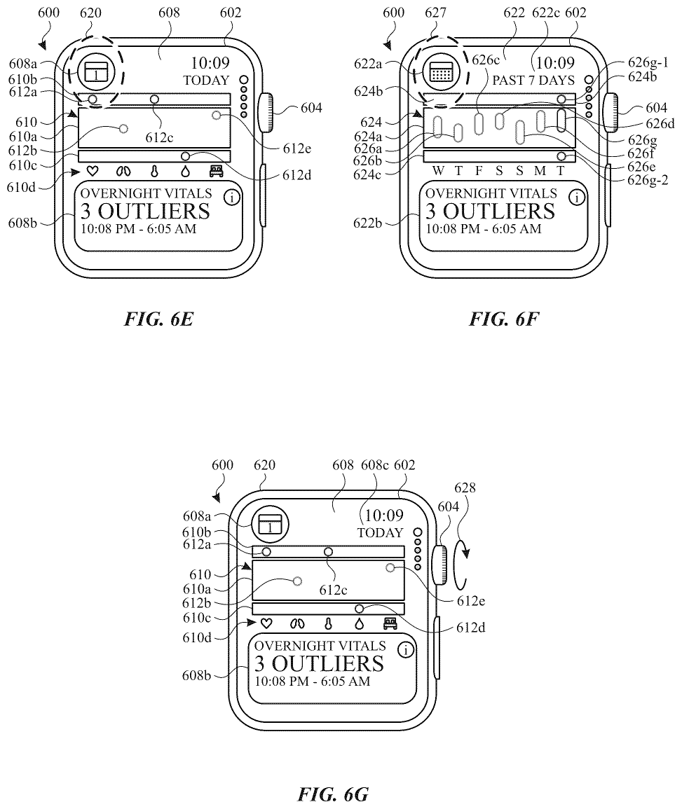

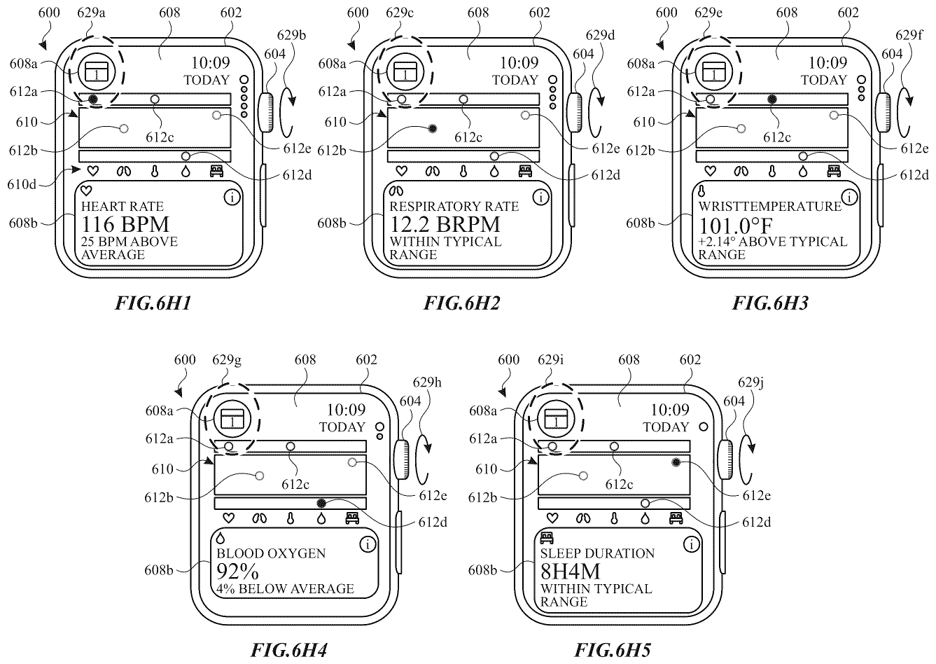

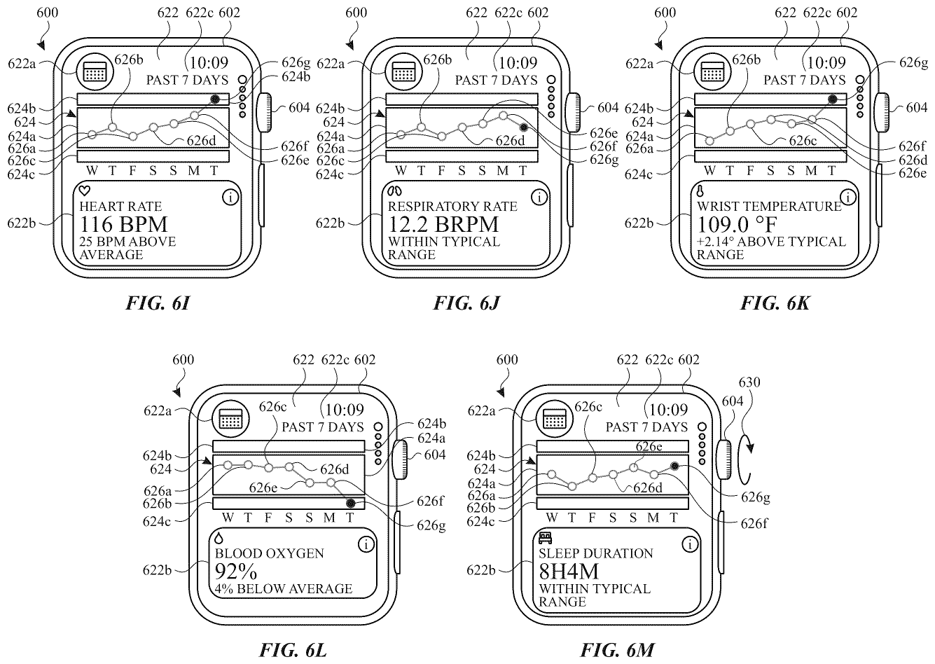

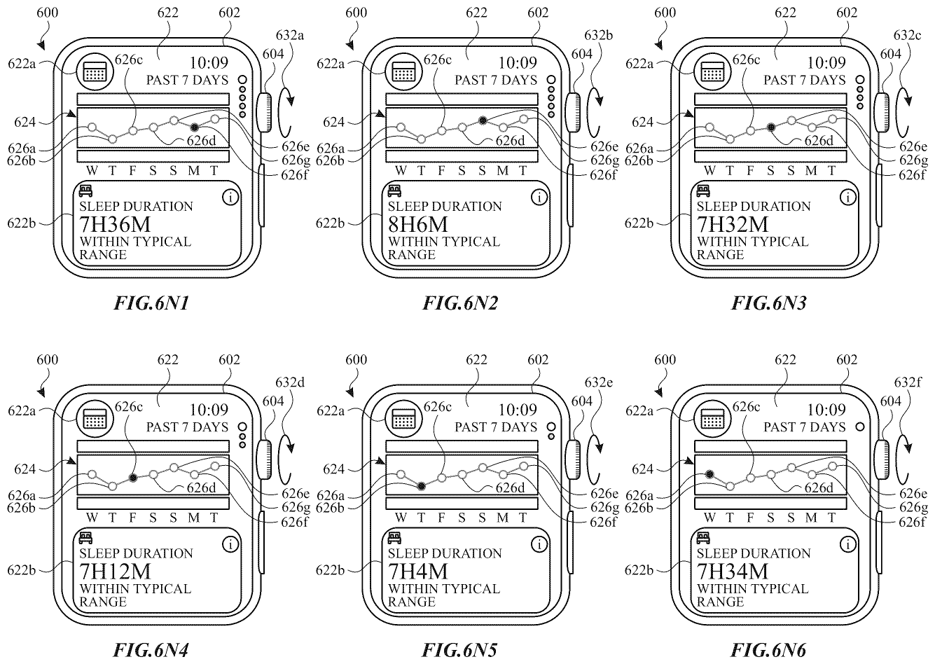

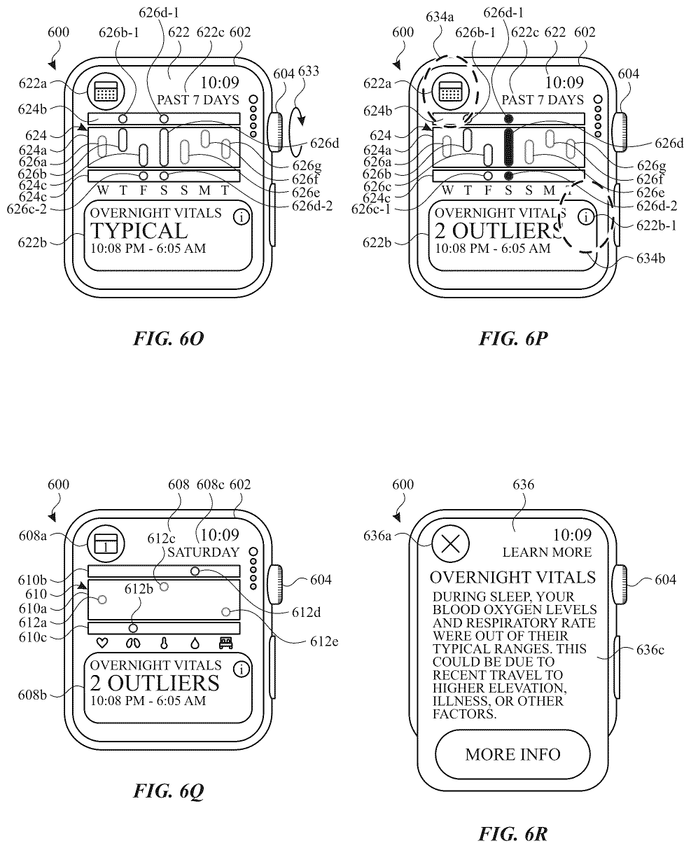

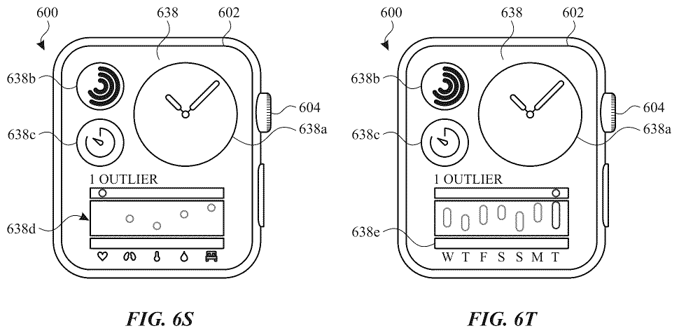

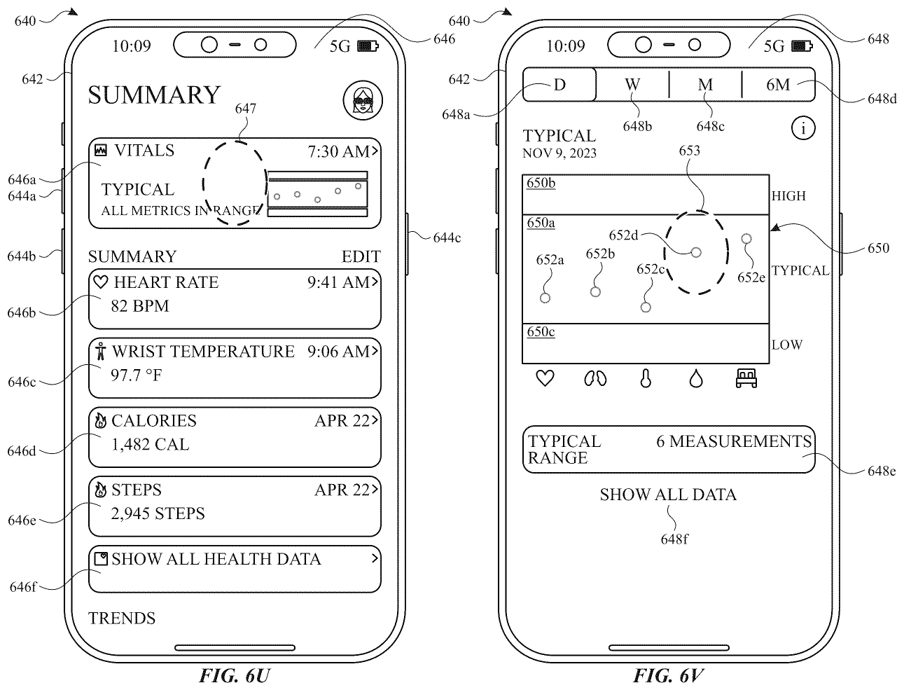

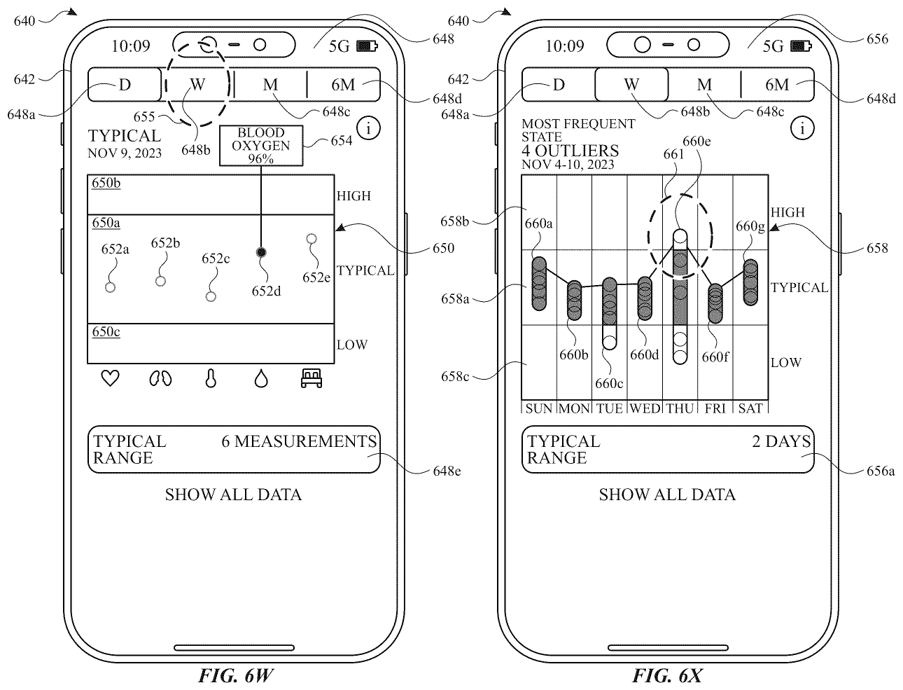

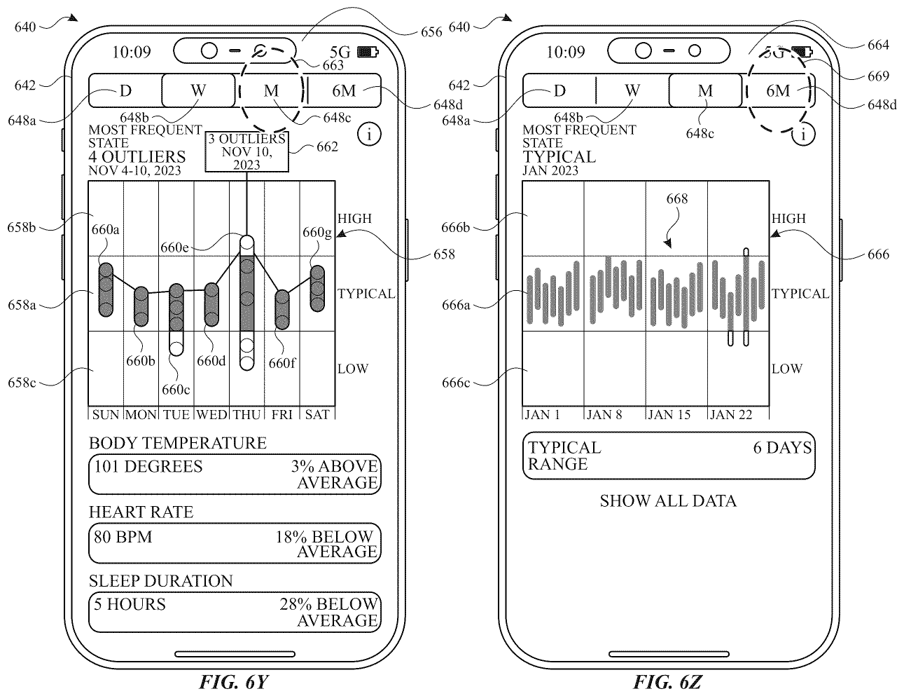

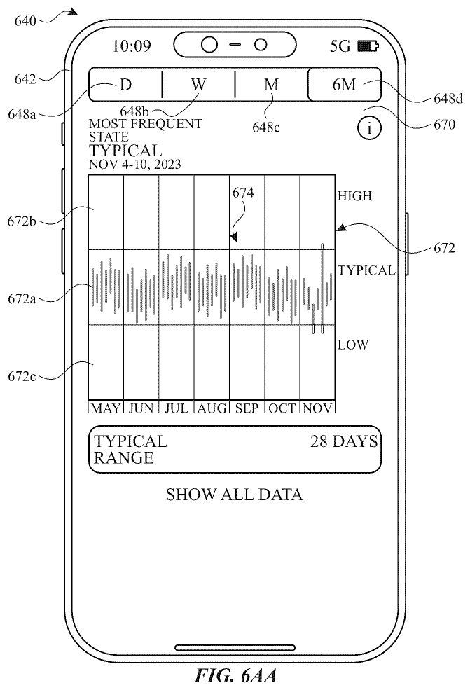

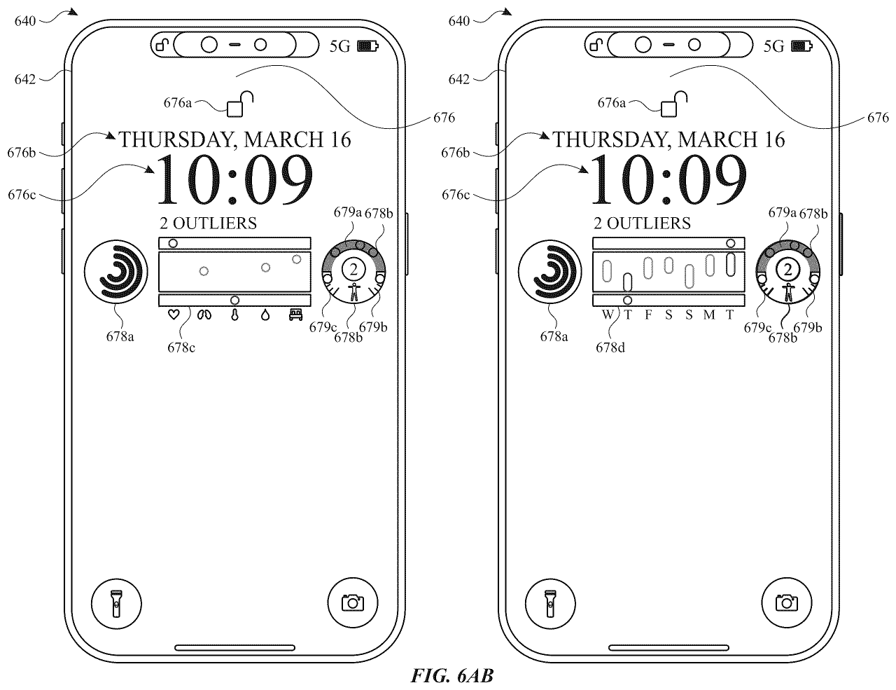

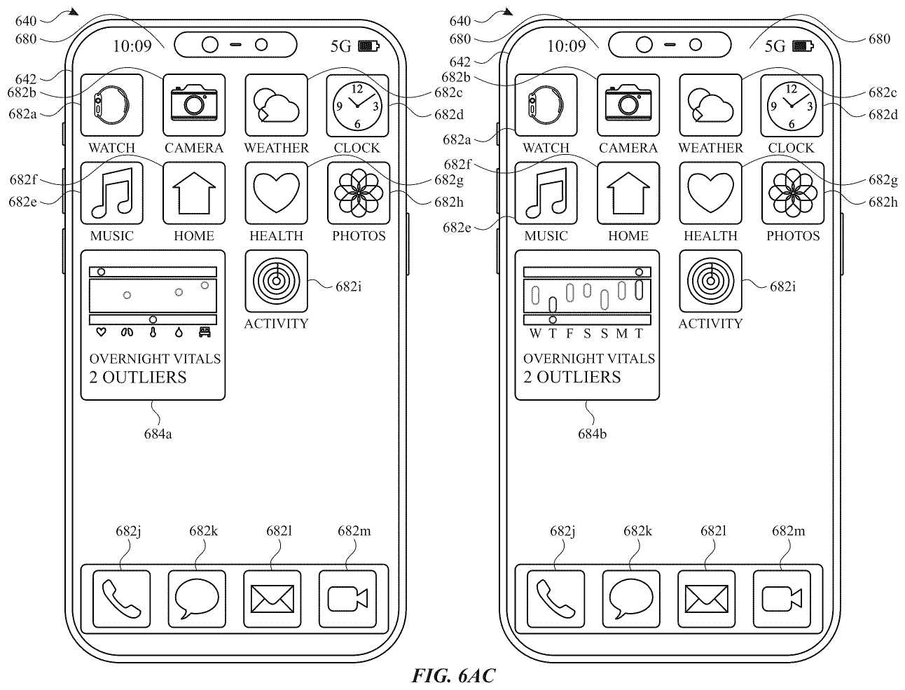

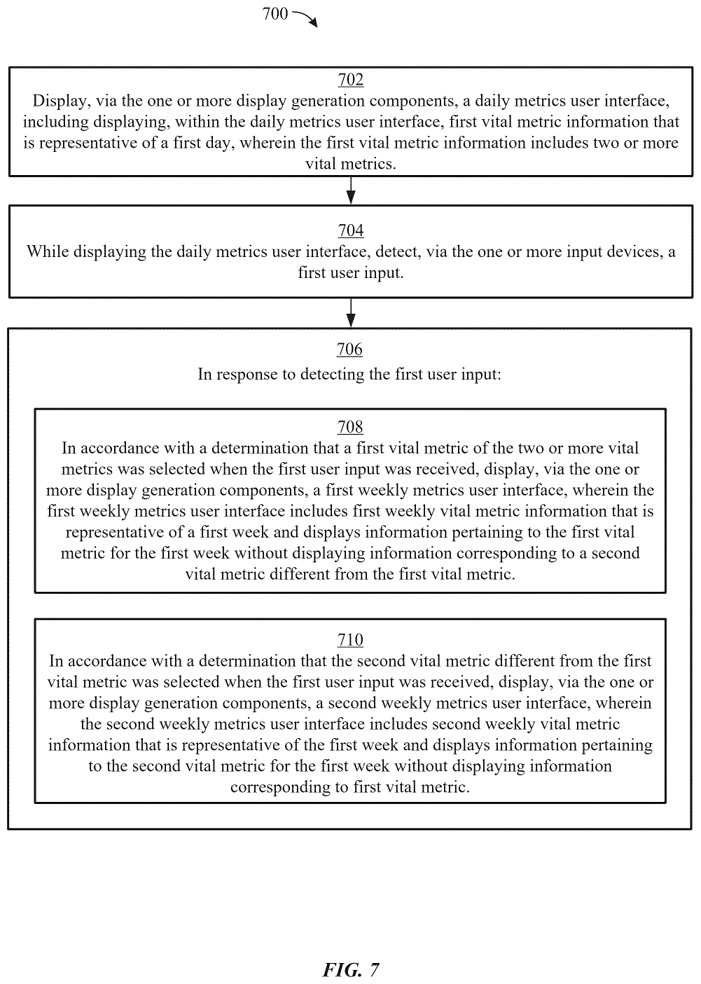

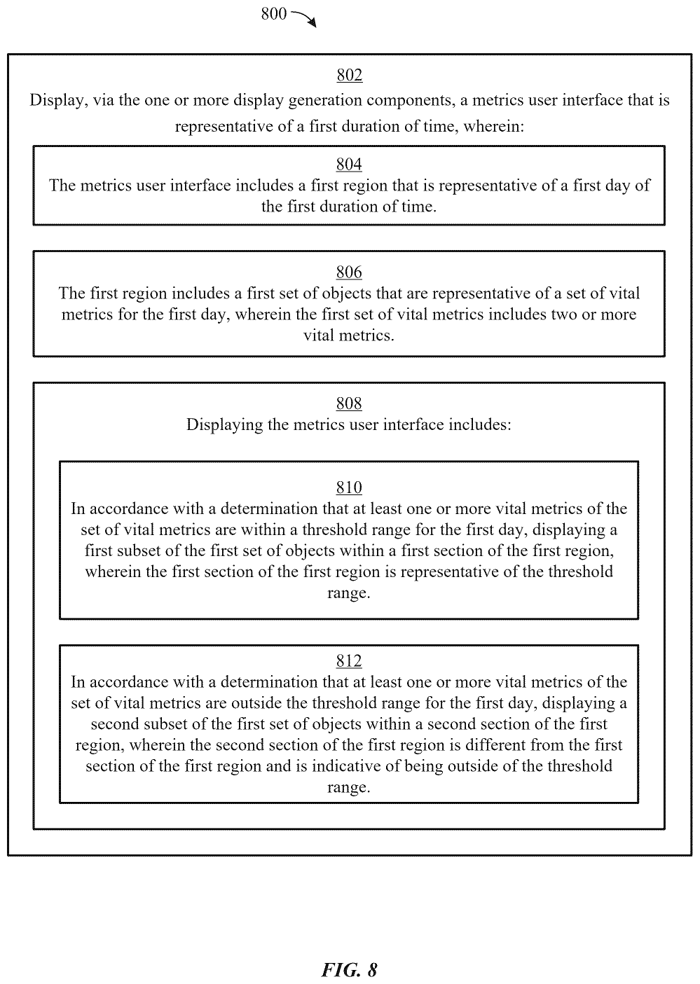

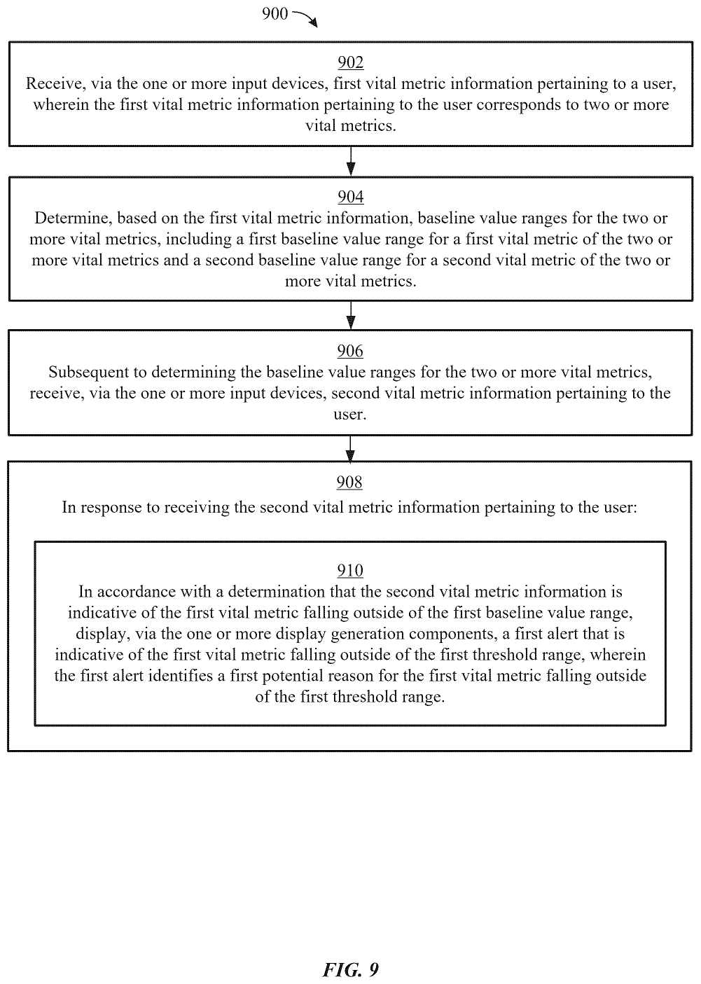

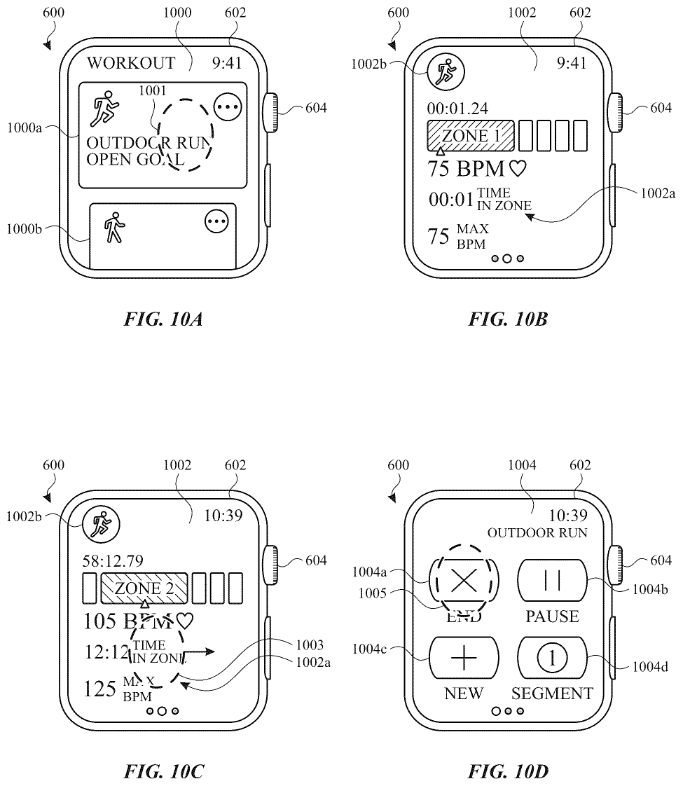

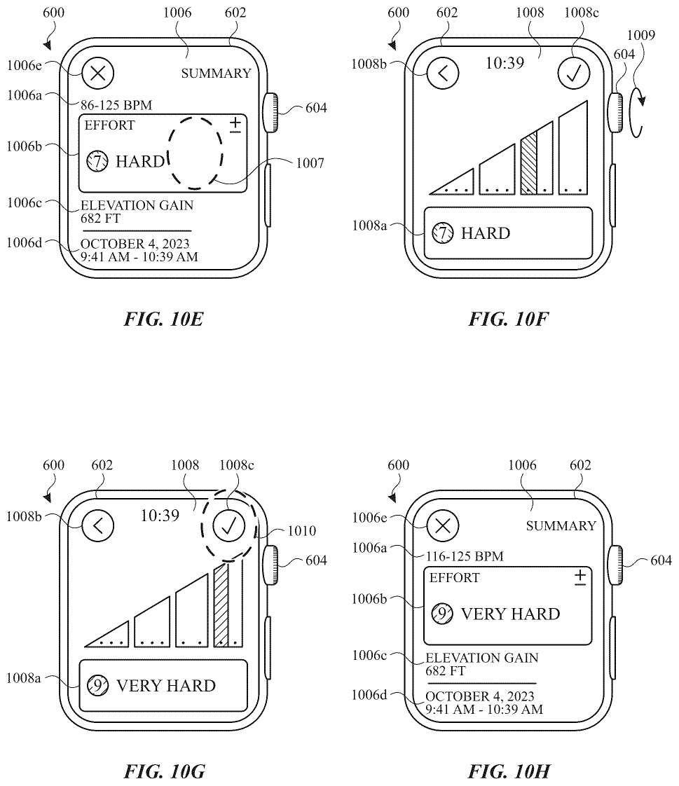

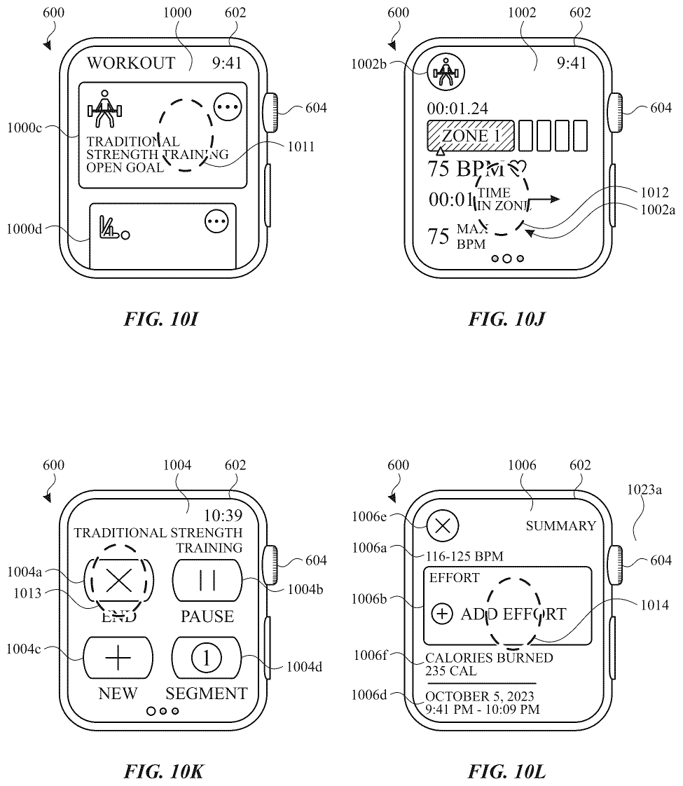

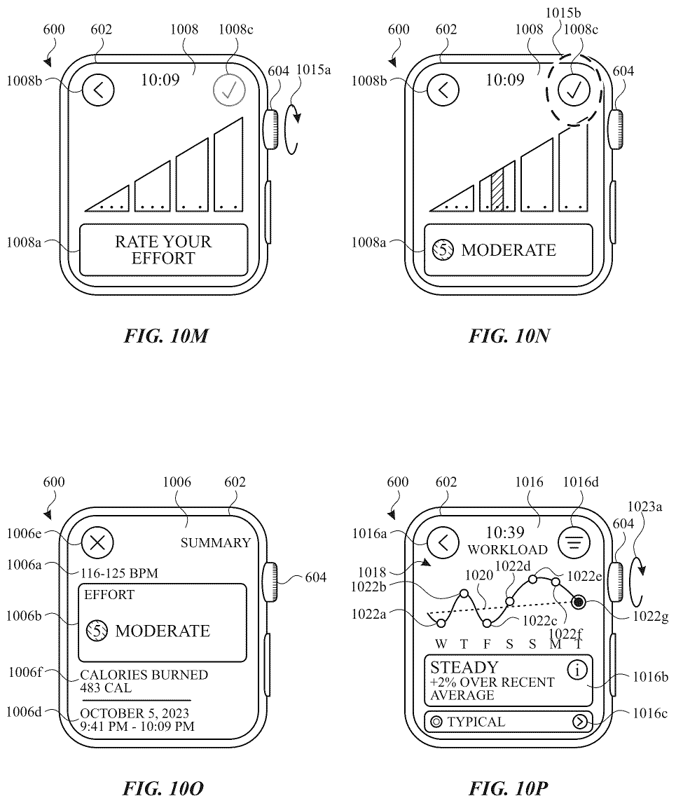

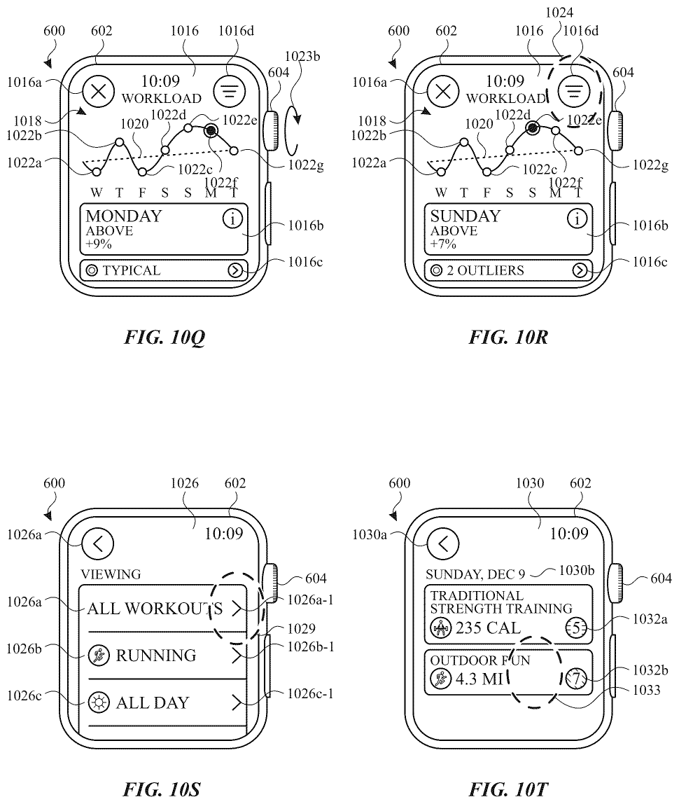

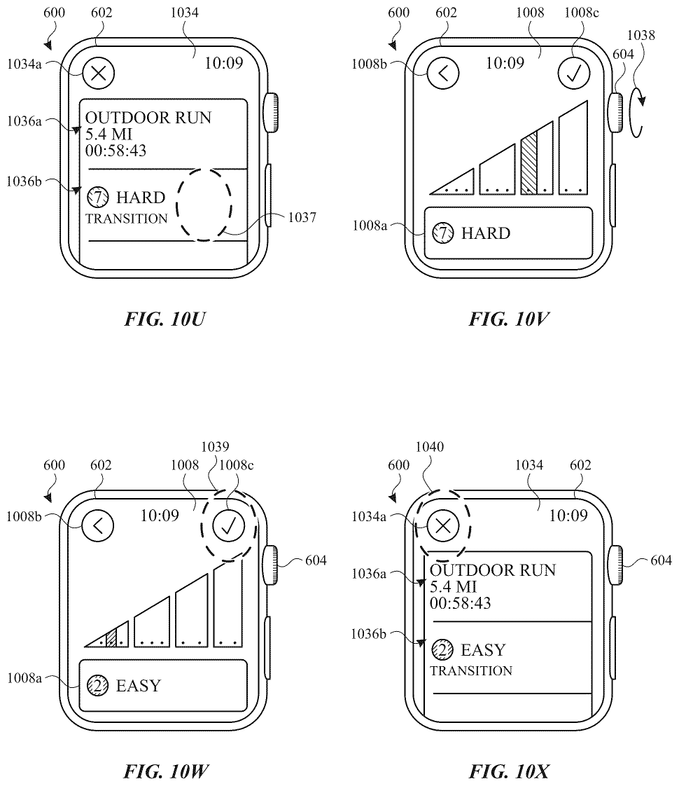

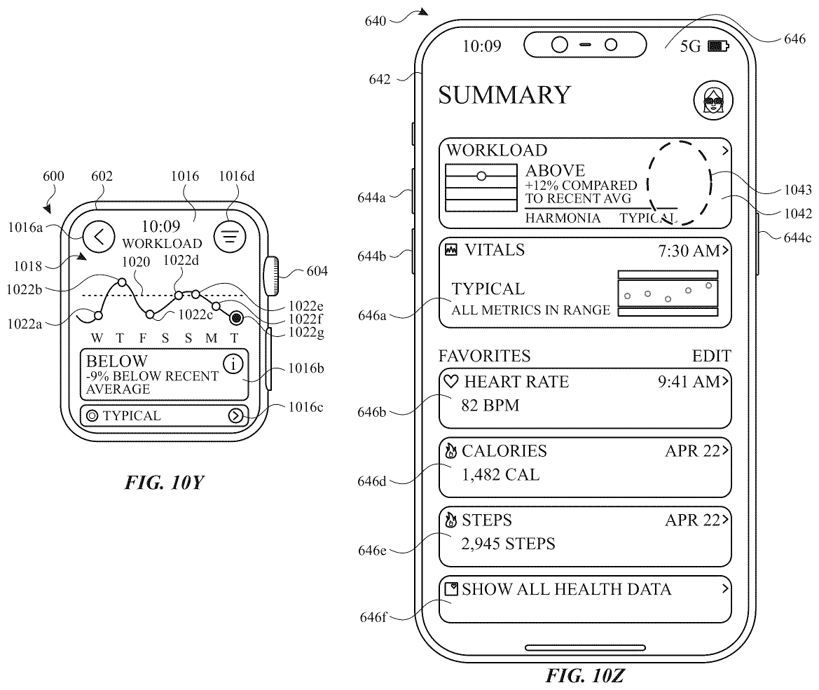

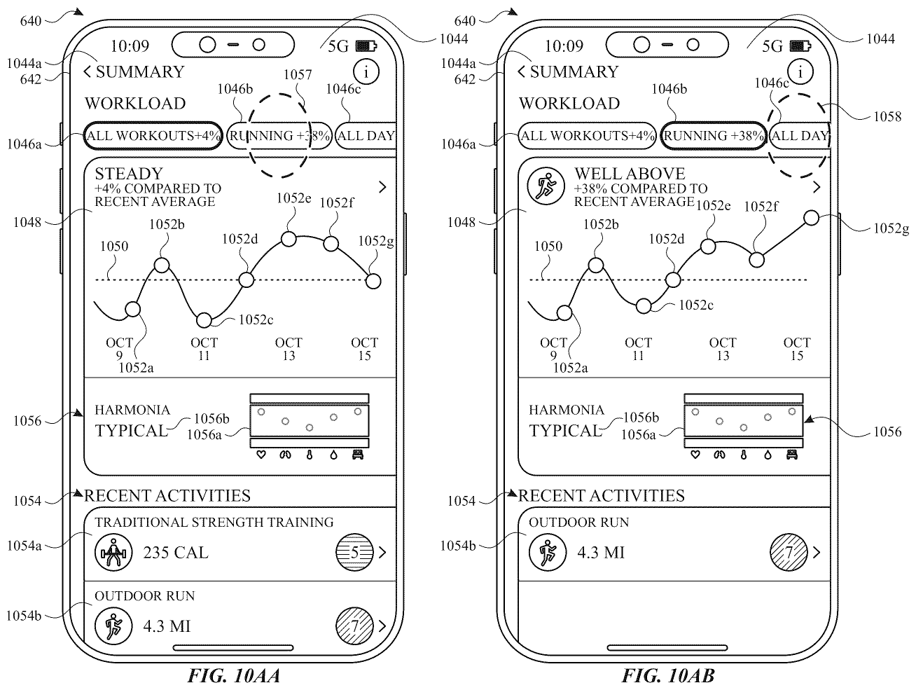

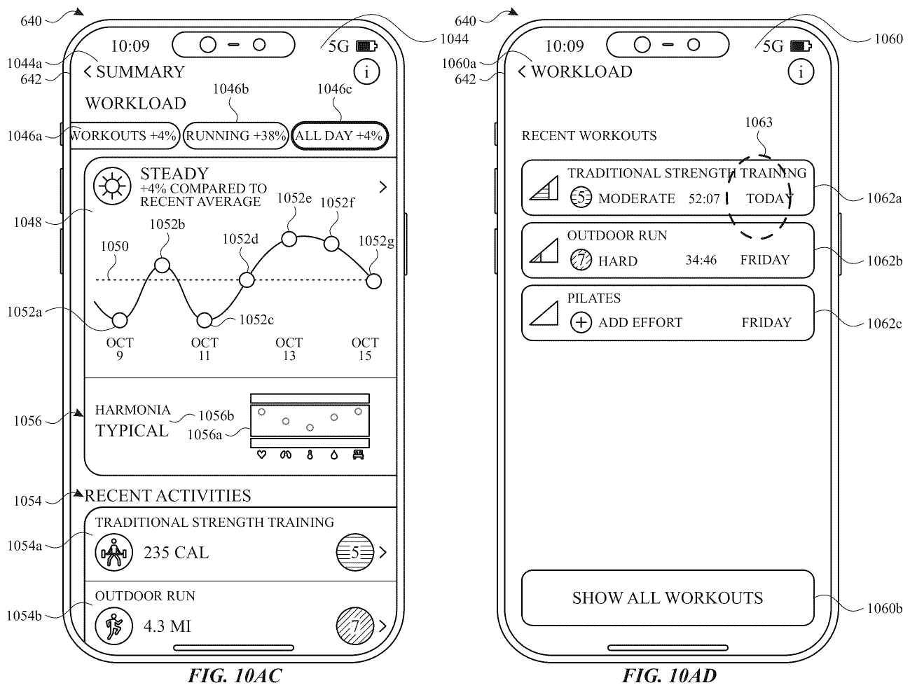

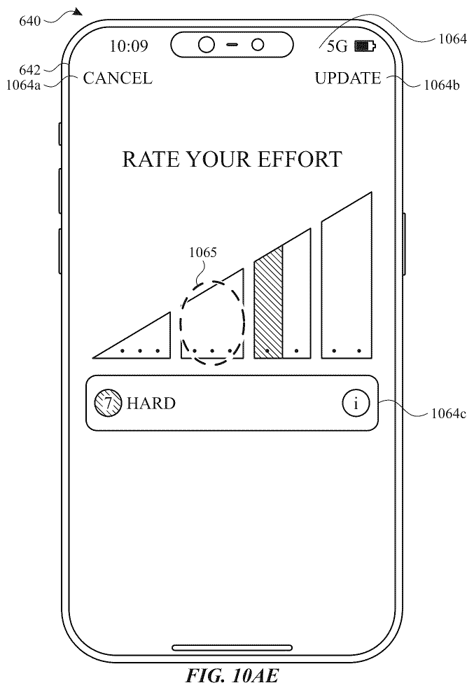

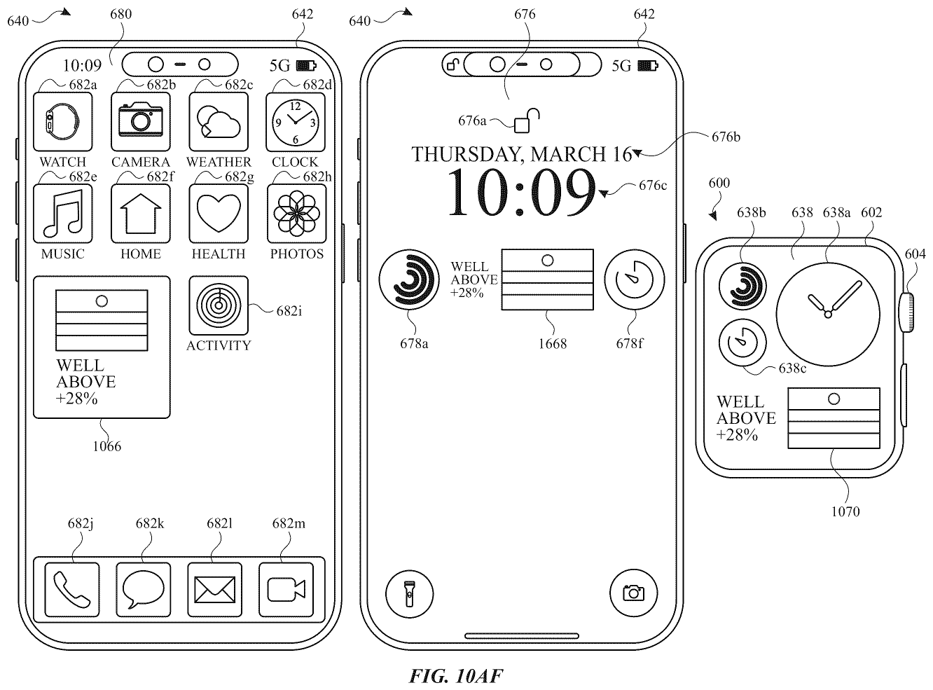

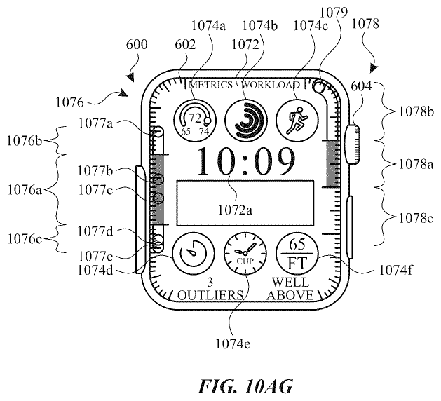

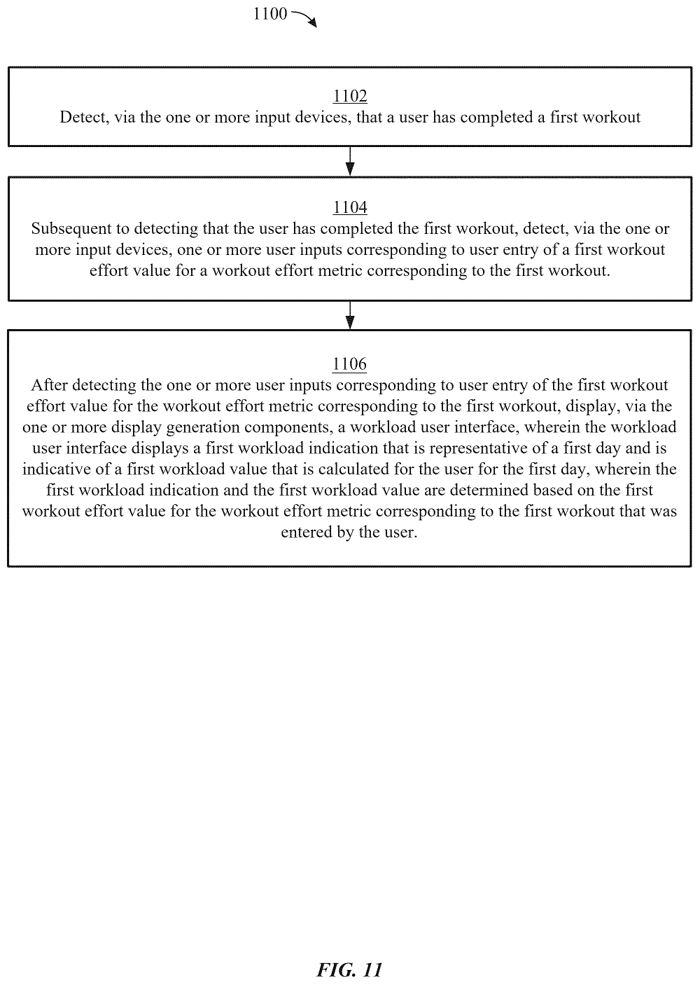

Below, FIGS. 1A-1B, 2, 3A-3G, 4A-4B, and 5A-5B provide a description of exemplary devices for performing the techniques for tracking and providing user health metrics. FIGS. 6A-6AC illustrate exemplary user interfaces for tracking and providing user health metrics. FIG. 7 is a flow diagram illustrating methods of tracking and providing user health metrics in accordance with some embodiments. FIG. 8 is a flow diagram illustrating methods of tracking and providing user health metrics in accordance with some embodiments. FIG. 9 is a flow diagram illustrating methods of tracking and providing user health metrics in accordance with some embodiments. The user interfaces in FIGS. 6A-6AC are used to illustrate the processes described below, including the processes in FIG. 7, FIG. 8, and FIG. 9. FIGS. 10A-10AG illustrate exemplary user interfaces for FIG. 7 is a flow diagram illustrating methods of tracking and providing user health metrics in accordance with some embodiments. FIG. 11 is a flow diagram illustrating methods of tracking and providing user health metrics in accordance with some embodiments. The user interfaces in FIGS. 10A-10AG are used to illustrate the processes described below, including the processes in FIG. 11.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

As used herein, the phrase “one or more of A and/or B” is construed to include all combinations of A and B, including, but not limited to: A individually without B; B individually without A; as well as a combination of A and B. The phrase “one or more of A, B, and/or C” is construed to include all combinations of A, B, and C, including, but not limited to: A individually without B and C; B individually without A and C; C individually without A and B; as well as any combinations of A, B, and/or C (e.g., A and B without C; A and C without B; B and C without A; and/or A, B, and C). Additionally, as used herein, the phrase “selected from the group consisting of A, B, C, and a combination thereof” and the phrase “at least one of A, B, and C” shall be construed to have the same meaning as the phrase “one or more of A, B, and/or C” as defined above. As used herein, the phrase “at least one of A, B, or C” and “one or more of A, B, or C” shall be construed to have the same meaning as the phrase “one or more of A, B, and/or C” as defined above. As used herein, the phrase “a combination including all of A, B, and C” is construed to include a combination of all the elements listed (e.g., a combination of A, B, and C).

The terminology used in the description of the various described embodiments herein is for the purpose of describing particular embodiments only and is not intended to be limiting.

As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component (e.g., a display device such as a head-mounted display (HMD), a display, a projector, a touch-sensitive display, or other device or component that presents visual content to a user, for example on or in the display generation component itself or produced from the display generation component and visible elsewhere). The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage media), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio.

The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11ac), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

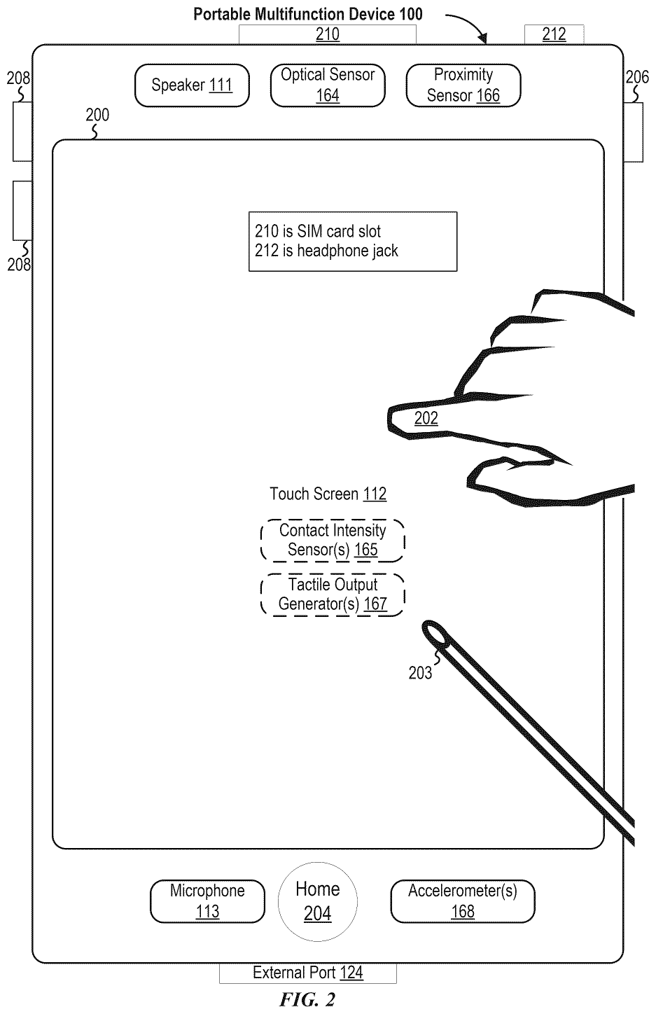

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both cars) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes secure element 163 for securely storing information. In some embodiments, secure element 163 is a hardware component (e.g., a secure microcontroller chip) configured to securely store data or an algorithm. In some embodiments, secure element 163 provides (e.g., releases) secure information (e.g., payment information (e.g., an account number and/or a transaction-specific dynamic security code), identification information (e.g., credentials of a state-approved digital identification), and/or authentication information (e.g., data generated using a cryptography engine and/or by performing asymmetric cryptography operations)). In some embodiments, secure element 163 provides (or releases) the secure information in response to device 100 receiving authorization, such as a user authentication (e.g., fingerprint authentication; passcode authentication; detecting double-press of a hardware button when device 100 is in an unlocked state, and optionally, while device 100 has been continuously on a user's wrist since device 100 was unlocked by providing authentication credentials to device 100, where the continuous presence of device 100 on the user's wrist is determined by periodically checking that the device is in contact with the user's skin). For example, device 100 detects a fingerprint at a fingerprint sensor (e.g., a fingerprint sensor integrated into a button) of device 100. Device 100 determines whether the detected fingerprint is consistent with an enrolled fingerprint. In accordance with a determination that the fingerprint is consistent with the enrolled fingerprint, secure element 163 provides (e.g., releases) the secure information. In accordance with a determination that the fingerprint is not consistent with the enrolled fingerprint, secure element 163 forgoes providing (e.g., releasing) the secure information.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

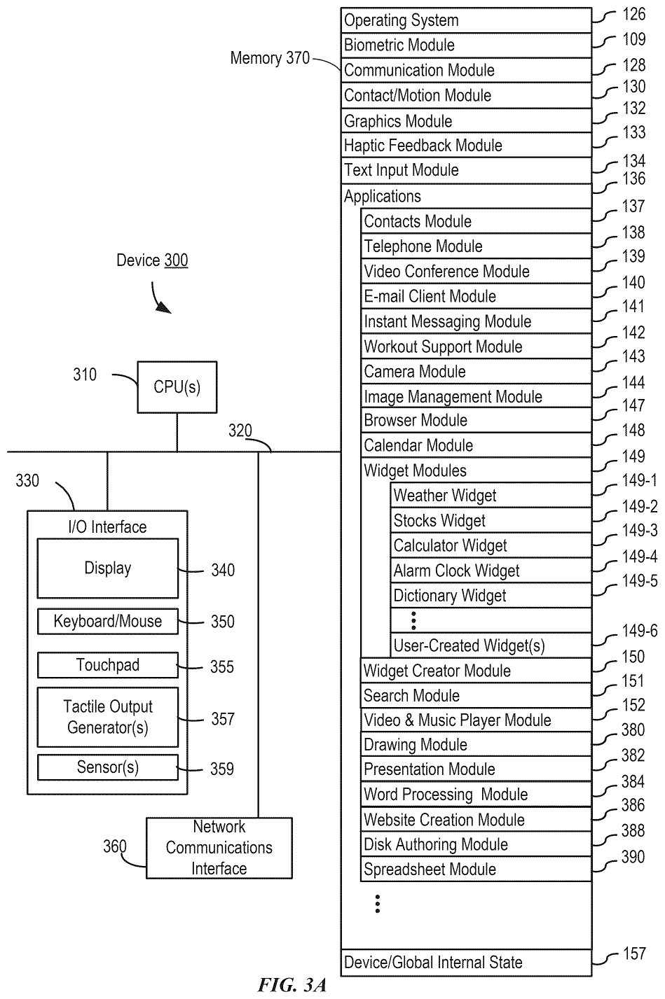

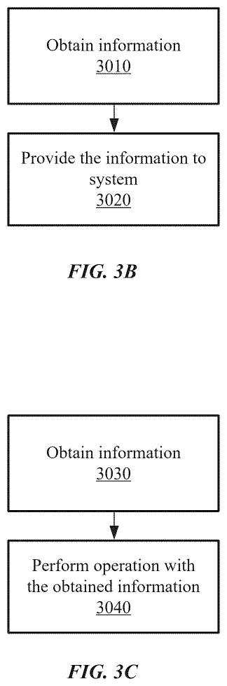

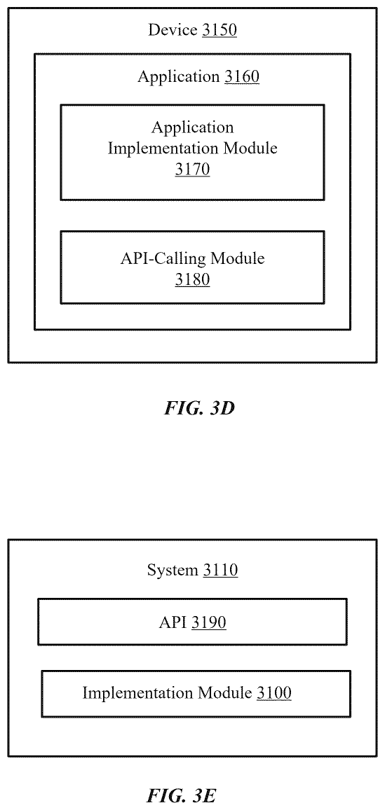

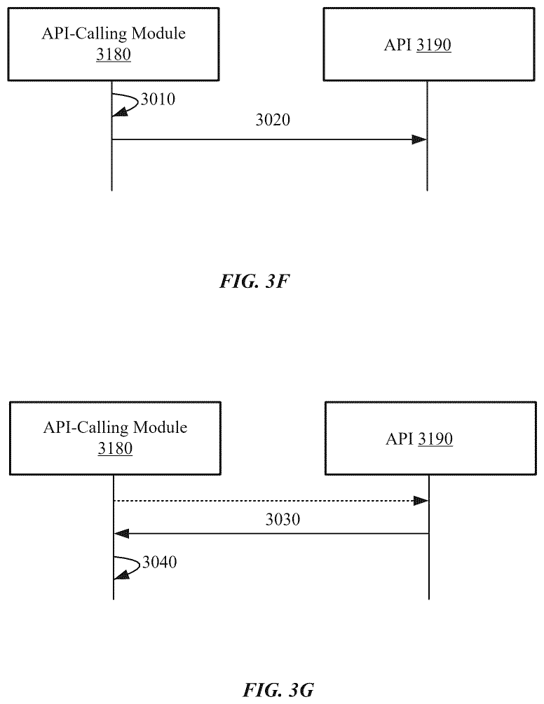

In some embodiments, the software components stored in memory 102 include operating system 126, biometric module 109, communication module (or set of instructions) 128, contact/motion module (or set of instructions) 130, graphics module (or set of instructions) 132, text input module (or set of instructions) 134, Global Positioning System (GPS) module (or set of instructions) 135, authentication module 105, and applications (or sets of instructions) 136. Furthermore, in some embodiments, memory 102 (FIG. 1A) or 370 (FIG. 3A) stores device/global internal state 157, as shown in FIGS. 1A and 3A. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, IOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE®, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Biometric module 109 optionally stores information about one or more enrolled biometric features (e.g., fingerprint feature information, facial recognition feature information, eye and/or iris feature information) for use to verify whether received biometric information matches the enrolled biometric features. In some embodiments, the information stored about the one or more enrolled biometric features includes data that enables the comparison between the stored information and received biometric information without including enough information to reproduce the enrolled biometric features. In some embodiments, biometric module 109 stores the information about the enrolled biometric features in association with a user account of device 100. In some embodiments, biometric module 109 compares the received biometric information to an enrolled biometric feature to determine whether the received biometric information matches the enrolled biometric feature.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts module 137, e-mail client module 140, IM module 141, browser module 147, and any other application that needs text input).

GPS module 135 determines the location of the device and provides this information for use in various applications (e.g., to telephone module 138 for use in location-based dialing; to camera module 143 as picture/video metadata; and to applications that provide location-based services such as weather widgets, local yellow page widgets, and map/navigation widgets).

Authentication module 105 determines whether a requested operation (e.g., requested by an application of applications 136) is authorized to be performed. In some embodiments, authentication module 105 receives for an operation to be perform that optionally requires authentication. Authentication module 105 determines whether the operation is authorized to be performed, such as based on a series of factors, including the lock status of device 100, the location of device 100, whether a security delay has elapsed, whether received biometric information matches enrolled biometric features, and/or other factors. Once authentication module 105 determines that the operation is authorized to be performed, authentication module 105 triggers performance of the operation.

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone module 138, video conference module 139, e-mail client module 140, or IM module 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript® file. In some embodiments, a widget includes an XML (Extensible Markup Language) file and a JavaScript® file (e.g., Yahoo!® Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3A) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3A is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3A is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Implementations within the scope of the present disclosure can be partially or entirely realized using a tangible computer-readable storage medium (or multiple tangible computer-readable storage media of one or more types) encoding one or more computer-readable instructions. It should be recognized that computer-readable instructions can be organized in any format, including applications, widgets, processes, software, and/or components.

Implementations within the scope of the present disclosure include a computer-readable storage medium that encodes instructions organized as an application (e.g., application 3160) that, when executed by one or more processing units, control an electronic device (e.g., device 3150) to perform the method of FIG. 3B, the method of FIG. 3C, and/or one or more other processes and/or methods described herein.

It should be recognized that application 3160 (shown in FIG. 3D) can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application. In some embodiments, application 3160 is an application that is pre-installed on device 3150 at purchase (e.g., a first-party application). In some embodiments, application 3160 is an application that is provided to device 3150 via an operating system update file (e.g., a first-party application or a second-party application). In some embodiments, application 3160 is an application that is provided via an application store. In some embodiments, the application store can be an application store that is pre-installed on device 3150 at purchase (e.g., a first-party application store). In some embodiments, the application store is a third-party application store (e.g., an application store that is provided by another application store, downloaded via a network, and/or read from a storage device).

Referring to FIG. 3B and FIG. 3F, application 3160 obtains information (e.g., 3010). In some embodiments, at 3010, information is obtained from at least one hardware component of device 3150. In some embodiments, at 3010, information is obtained from at least one software module of device 3150. In some embodiments, at 3010, information is obtained from at least one hardware component external to device 3150 (e.g., a peripheral device, an accessory device, and/or a server). In some embodiments, the information obtained at 3010 includes positional information, time information, notification information, user information, environment information, electronic device state information, weather information, media information, historical information, event information, hardware information, and/or motion information. In some embodiments, in response to and/or after obtaining the information at 3010, application 3160 provides the information to a system (e.g., 3020).

In some embodiments, the system (e.g., 3110 shown in FIG. 3E) is an operating system hosted on device 3150. In some embodiments, the system (e.g., 3110 shown in FIG. 3E) is an external device (e.g., a server, a peripheral device, an accessory, and/or a personal computing device) that includes an operating system.

Referring to FIG. 3C and FIG. 3G, application 3160 obtains information (e.g., 3030). In some embodiments, the information obtained at 3030 includes positional information, time information, notification information, user information, environment information electronic device state information, weather information, media information, historical information, event information, hardware information, and/or motion information. In response to and/or after obtaining the information at 3030, application 3160 performs an operation with the information (e.g., 3040). In some embodiments, the operation performed at 3040 includes: providing a notification based on the information, sending a message based on the information, displaying the information, controlling a user interface of a fitness application based on the information, controlling a user interface of a health application based on the information, controlling a focus mode based on the information, setting a reminder based on the information, adding a calendar entry based on the information, and/or calling an API of system 3110 based on the information.

In some embodiments, one or more steps of the method of FIG. 3B and/or the method of FIG. 3C is performed in response to a trigger. In some embodiments, the trigger includes detection of an event, a notification received from system 3110, a user input, and/or a response to a call to an API provided by system 3110.

In some embodiments, the instructions of application 3160, when executed, control device 3150 to perform the method of FIG. 3B and/or the method of FIG. 3C by calling an application programming interface (API) (e.g., API 3190) provided by system 3110. In some embodiments, application 3160 performs at least a portion of the method of FIG. 3B and/or the method of FIG. 3C without calling API 3190.

In some embodiments, one or more steps of the method of FIG. 3B and/or the method of FIG. 3C includes calling an API (e.g., API 3190) using one or more parameters defined by the API. In some embodiments, the one or more parameters include a constant, a key, a data structure, an object, an object class, a variable, a data type, a pointer, an array, a list or a pointer to a function or method, and/or another way to reference a data or other item to be passed via the API.

Referring to FIG. 3D, device 3150 is illustrated. In some embodiments, device 3150 is a personal computing device, a smart phone, a smart watch, a fitness tracker, a head mounted display (HMD) device, a media device, a communal device, a speaker, a television, and/or a tablet. As illustrated in FIG. 3D, device 3150 includes application 3160 and an operating system (e.g., system 3110 shown in FIG. 3E). Application 3160 includes application implementation module 3170 and API-calling module 3180. System 3110 includes API 3190 and implementation module 3100. It should be recognized that device 3150, application 3160, and/or system 3110 can include more, fewer, and/or different components than illustrated in FIGS. 3D and 3E.

In some embodiments, application implementation module 3170 includes a set of one or more instructions corresponding to one or more operations performed by application 3160. For example, when application 3160 is a messaging application, application implementation module 3170 can include operations to receive and send messages. In some embodiments, application implementation module 3170 communicates with API-calling module 3180 to communicate with system 3110 via API 3190 (shown in FIG. 3E).

In some embodiments, API 3190 is a software module (e.g., a collection of computer-readable instructions) that provides an interface that allows a different module (e.g., API-calling module 3180) to access and/or use one or more functions, methods, procedures, data structures, classes, and/or other services provided by implementation module 3100 of system 3110. For example, API-calling module 3180 can access a feature of implementation module 3100 through one or more API calls or invocations (e.g., embodied by a function or a method call) exposed by API 3190 (e.g., a software and/or hardware module that can receive API calls, respond to API calls, and/or send API calls) and can pass data and/or control information using one or more parameters via the API calls or invocations. In some embodiments, API 3190 allows application 3160 to use a service provided by a Software Development Kit (SDK) library. In some embodiments, application 3160 incorporates a call to a function or method provided by the SDK library and provided by API 3190 or uses data types or objects defined in the SDK library and provided by API 3190. In some embodiments, API-calling module 3180 makes an API call via API 3190 to access and use a feature of implementation module 3100 that is specified by API 3190. In such embodiments, implementation module 3100 can return a value via API 3190 to API-calling module 3180 in response to the API call. The value can report to application 3160 the capabilities or state of a hardware component of device 3150, including those related to aspects such as input capabilities and state, output capabilities and state, processing capability, power state, storage capacity and state, and/or communications capability. In some embodiments, API 3190 is implemented in part by firmware, microcode, or other low level logic that executes in part on the hardware component.

In some embodiments, API 3190 allows a developer of API-calling module 3180 (which can be a third-party developer) to leverage a feature provided by implementation module 3100. In such embodiments, there can be one or more API-calling modules (e.g., including API-calling module 3180) that communicate with implementation module 3100. In some embodiments, API 3190 allows multiple API-calling modules written in different programming languages to communicate with implementation module 3100 (e.g., API 3190 can include features for translating calls and returns between implementation module 3100 and API-calling module 3180) while API 3190 is implemented in terms of a specific programming language. In some embodiments, API-calling module 3180 calls APIs from different providers such as a set of APIs from an OS provider, another set of APIs from a plug-in provider, and/or another set of APIs from another provider (e.g., the provider of a software library) or creator of the another set of APIs.

Examples of API 3190 can include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, photos API, camera API, and/or image processing API. In some embodiments, the sensor API is an API for accessing data associated with a sensor of device 3150. For example, the sensor API can provide access to raw sensor data. For another example, the sensor API can provide data derived (and/or generated) from the raw sensor data. In some embodiments, the sensor data includes temperature data, image data, video data, audio data, heart rate data, IMU (inertial measurement unit) data, lidar data, location data, GPS data, and/or camera data. In some embodiments, the sensor includes one or more of an accelerometer, temperature sensor, infrared sensor, optical sensor, heartrate sensor, barometer, gyroscope, proximity sensor, temperature sensor, and/or biometric sensor.

In some embodiments, implementation module 3100 is a system (e.g., operating system and/or server system) software module (e.g., a collection of computer-readable instructions) that is constructed to perform an operation in response to receiving an API call via API 3190. In some embodiments, implementation module 3100 is constructed to provide an API response (via API 3190) as a result of processing an API call. By way of example, implementation module 3100 and API-calling module 3180 can each be any one of an operating system, a library, a device driver, an API, an application program, or other module. It should be understood that implementation module 3100 and API-calling module 3180 can be the same or different type of module from each other. In some embodiments, implementation module 3100 is embodied at least in part in firmware, microcode, or hardware logic.

In some embodiments, implementation module 3100 returns a value through API 3190 in response to an API call from API-calling module 3180. While API 3190 defines the syntax and result of an API call (e.g., how to invoke the API call and what the API call does), API 3190 might not reveal how implementation module 3100 accomplishes the function specified by the API call. Various API calls are transferred via the one or more application programming interfaces between API-calling module 3180 and implementation module 3100. Transferring the API calls can include issuing, initiating, invoking, calling, receiving, returning, and/or responding to the function calls or messages. In other words, transferring can describe actions by either of API-calling module 3180 or implementation module 3100. In some embodiments, a function call or other invocation of API 3190 sends and/or receives one or more parameters through a parameter list or other structure.

In some embodiments, implementation module 3100 provides more than one API, each providing a different view of or with different aspects of functionality implemented by implementation module 3100. For example, one API of implementation module 3100 can provide a first set of functions and can be exposed to third-party developers, and another API of implementation module 3100 can be hidden (e.g., not exposed) and provide a subset of the first set of functions and also provide another set of functions, such as testing or debugging functions which are not in the first set of functions. In some embodiments, implementation module 3100 calls one or more other components via an underlying API and thus is both an API-calling module and an implementation module. It should be recognized that implementation module 3100 can include additional functions, methods, classes, data structures, and/or other features that are not specified through API 3190 and are not available to API-calling module 3180. It should also be recognized that API-calling module 3180 can be on the same system as implementation module 3100 or can be located remotely and access implementation module 3100 using API 3190 over a network. In some embodiments, implementation module 3100, API 3190, and/or API-calling module 3180 is stored in a machine-readable medium, which includes any mechanism for storing information in a form readable by a machine (e.g., a computer or other data processing system). For example, a machine-readable medium can include magnetic disks, optical disks, random access memory; read only memory, and/or flash memory devices.

An application programming interface (API) is an interface between a first software process and a second software process that specifies a format for communication between the first software process and the second software process. Limited APIs (e.g., private APIs or partner APIs) are APIs that are accessible to a limited set of software processes (e.g., only software processes within an operating system or only software processes that are approved to access the limited APIs). Public APIs that are accessible to a wider set of software processes. Some APIs enable software processes to communicate about or set a state of one or more input devices (e.g., one or more touch sensors, proximity sensors, visual sensors, motion/orientation sensors, pressure sensors, intensity sensors, sound sensors, wireless proximity sensors, biometric sensors, buttons, switches, rotatable elements, and/or external controllers). Some APIs enable software processes to communicate about and/or set a state of one or more output generation components (e.g., one or more audio output generation components, one or more display generation components, and/or one or more tactile output generation components). Some APIs enable particular capabilities (e.g., scrolling, handwriting, text entry, image editing, and/or image creation) to be accessed, performed, and/or used by a software process (e.g., generating outputs for use by a software process based on input from the software process). Some APIs enable content from a software process to be inserted into a template and displayed in a user interface that has a layout and/or behaviors that are specified by the template.

Many software platforms include a set of frameworks that provides the core objects and core behaviors that a software developer needs to build software applications that can be used on the software platform. Software developers use these objects to display content onscreen, to interact with that content, and to manage interactions with the software platform. Software applications rely on the set of frameworks for their basic behavior, and the set of frameworks provides many ways for the software developer to customize the behavior of the application to match the specific needs of the software application. Many of these core objects and core behaviors are accessed via an API. An API will typically specify a format for communication between software processes, including specifying and grouping available variables, functions, and protocols. An API call (sometimes referred to as an API request) will typically be sent from a sending software process to a receiving software process as a way to accomplish one or more of the following: the sending software process requesting information from the receiving software process (e.g., for the sending software process to take action on), the sending software process providing information to the receiving software process (e.g., for the receiving software process to take action on), the sending software process requesting action by the receiving software process, or the sending software process providing information to the receiving software process about action taken by the sending software process. Interaction with a device (e.g., using a user interface) will in some circumstances include the transfer and/or receipt of one or more API calls (e.g., multiple API calls) between multiple different software processes (e.g., different portions of an operating system, an application and an operating system, or different applications) via one or more APIs (e.g., via multiple different APIs). For example, when an input is detected the direct sensor data is frequently processed into one or more input events that are provided (e.g., via an API) to a receiving software process that makes some determination based on the input events, and then sends (e.g., via an API) information to a software process to perform an operation (e.g., change a device state and/or user interface) based on the determination. While a determination and an operation performed in response could be made by the same software process, alternatively the determination could be made in a first software process and relayed (e.g., via an API) to a second software process, that is different from the first software process, that causes the operation to be performed by the second software process. Alternatively, the second software process could relay instructions (e.g., via an API) to a third software process that is different from the first software process and/or the second software process to perform the operation. It should be understood that some or all user interactions with a computer system could involve one or more API calls within a step of interacting with the computer system (e.g., between different software components of the computer system or between a software component of the computer system and a software component of one or more remote computer systems). It should be understood that some or all user interactions with a computer system could involve one or more API calls between steps of interacting with the computer system (e.g., between different software components of the computer system or between a software component of the computer system and a software component of one or more remote computer systems).

In some embodiments, the application can be any suitable type of application, including, for example, one or more of: a browser application, an application that functions as an execution environment for plug-ins, widgets or other applications, a fitness application, a health application, a digital payments application, a media application, a social network application, a messaging application, and/or a maps application.

In some embodiments, the application is an application that is pre-installed on the first computer system at purchase (e.g., a first-party application). In some embodiments, the application is an application that is provided to the first computer system via an operating system update file (e.g., a first-party application). In some embodiments, the application is an application that is provided via an application store. In some embodiments, the application store is pre-installed on the first computer system at purchase (e.g., a first-party application store) and allows download of one or more applications. In some embodiments, the application store is a third-party application store (e.g., an application store that is provided by another device, downloaded via a network, and/or read from a storage device). In some embodiments, the application is a third-party application (e.g., an app that is provided by an application store, downloaded via a network, and/or read from a storage device). In some embodiments, the application controls the first computer system to perform method 700 (FIG. 7), method 800 (FIG. 8), method 900 (FIG. 9), and/or method 1100 (FIG. 11) by calling an application programming interface (API) provided by the system process using one or more parameters.

In some embodiments, exemplary APIs provided by the system process include one or more of: a pairing API (e.g., for establishing secure connection, e.g., with an accessory), a device detection API (e.g., for locating nearby devices, e.g., media devices and/or smartphone), a payment API, a UIKit API (e.g., for generating user interfaces), a location detection API, a locator API, a maps API, a health sensor API, a sensor API, a messaging API, a push notification API, a streaming API, a collaboration API, a video conferencing API, an application store API, an advertising services API, a web browser API (e.g., WebKit API), a vehicle API, a networking API, a WiFi API, a Bluetooth API, an NFC API, a UWB API, a fitness API, a smart home API, contact transfer API, a photos API, a camera API, and/or an image processing API.

In some embodiments, at least one API is a software module (e.g., a collection of computer-readable instructions) that provides an interface that allows a different module (e.g., API-calling module 3180) to access and use one or more functions, methods, procedures, data structures, classes, and/or other services provided by an implementation module of the system process. The API can define one or more parameters that are passed between the API-calling module and the implementation module. In some embodiments, API 3190 defines a first API call that can be provided by API-calling module 3180. The implementation module is a system software module (e.g., a collection of computer-readable instructions) that is constructed to perform an operation in response to receiving an API call via the API. In some embodiments, the implementation module is constructed to provide an API response (via the API) as a result of processing an API call. In some embodiments, the implementation module is included in the device (e.g., 3150) that runs the application. In some embodiments, the implementation module is included in an electronic device that is separate from the device that runs the application.

Attention is now directed towards embodiments of user interfaces that are, optionally, implemented on, for example, portable multifunction device 100.

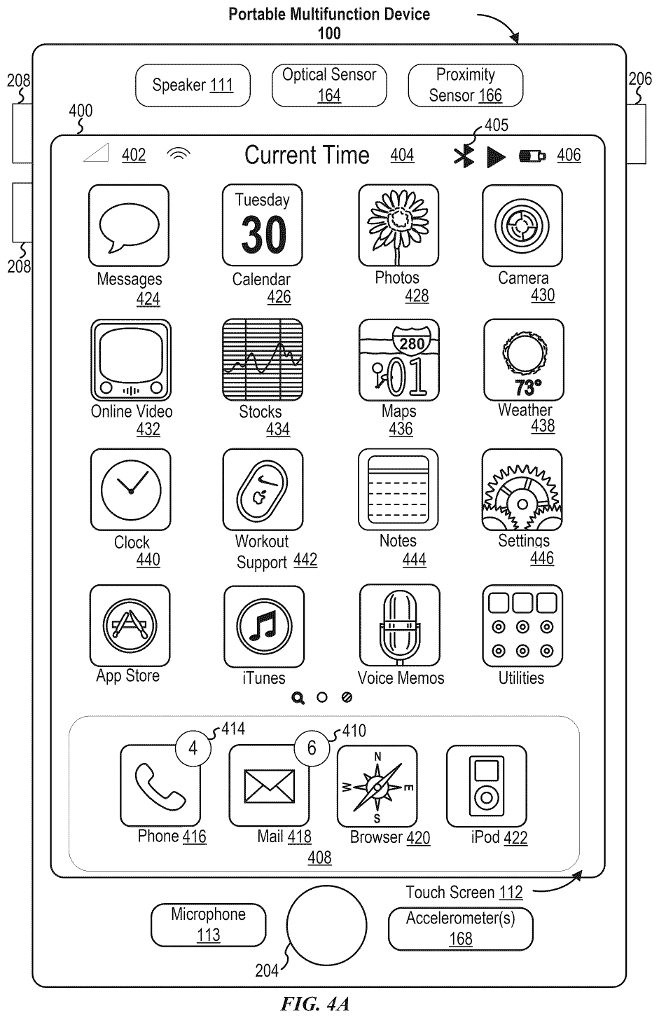

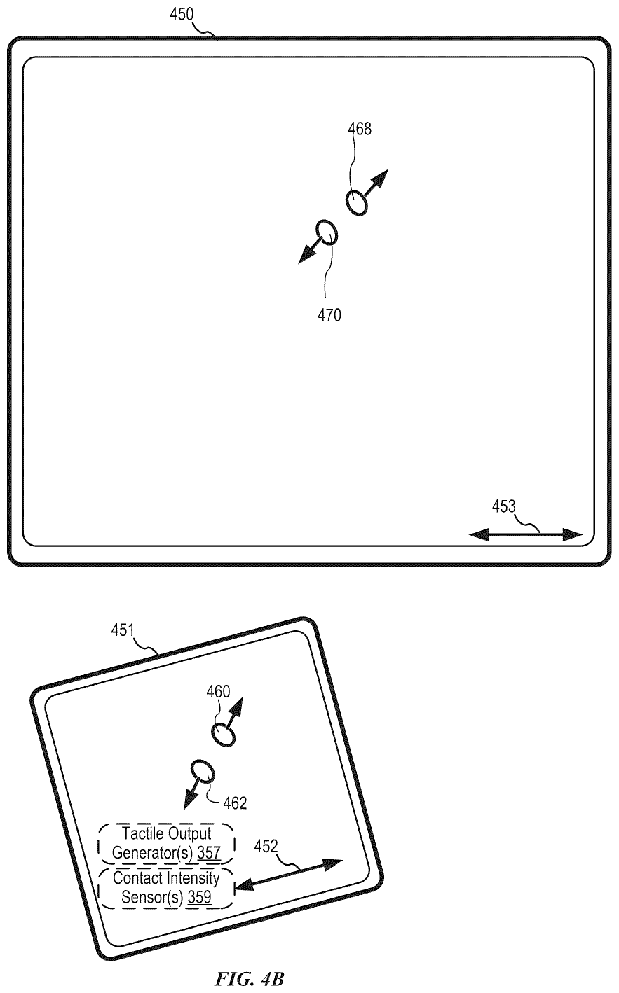

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3A) with a touch-sensitive surface 451 (e.g., a tablet or touchpad 355, FIG. 3A) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., touch-sensitive surface 451 in FIG. 4B) has a primary axis (e.g., 452 in FIG. 4B) that corresponds to a primary axis (e.g., 453 in FIG. 4B) on the display (e.g., display 450). In accordance with these embodiments, the device detects contacts (e.g., contact 460 and contact 462 in FIG. 4B) with the touch-sensitive surface 451 at locations that correspond to respective locations on the display (e.g., in FIG. 4B, contact 460 corresponds to 468 and contact 462 corresponds to 470). In this way, user inputs (e.g., contacts 460 and 462, and movements thereof) detected by the device on the touch-sensitive surface (e.g., touch-sensitive surface 451 in FIG. 4B) are used by the device to manipulate the user interface on the display (e.g., display 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

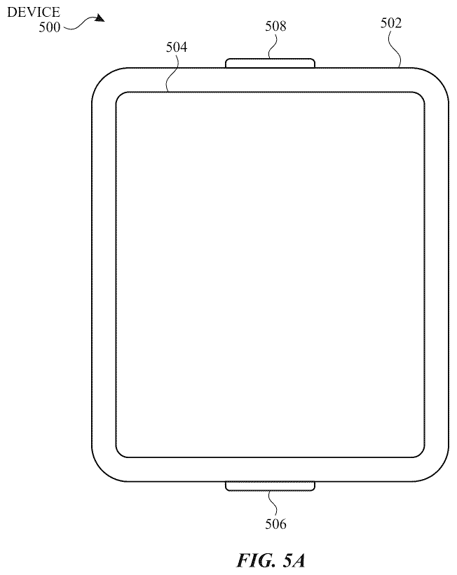

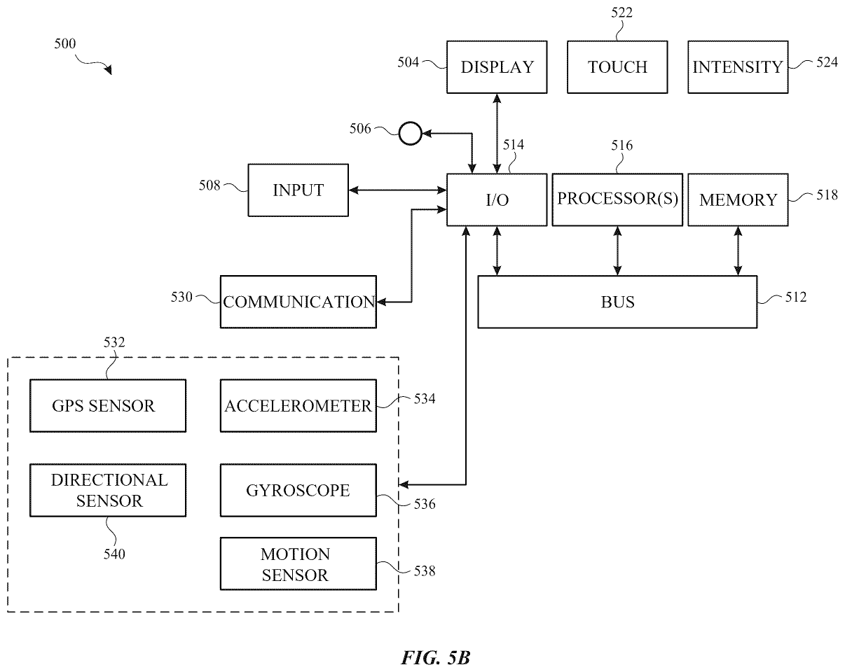

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1B, and 3A. Device 500 has bus 512 that operatively couples I/O section 514 with one or more computer processors 516 and memory 518. I/O section 514 can be connected to display screen 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage media, for storing computer-executable instructions, which, when executed by one or more computer processors 516, for example, can cause the computer processors to perform the techniques described below, including processes 700-900 and 1100 (FIGS. 7, 8, 9, and 11). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3A, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3A or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

As used herein, an “installed application” refers to a software application that has been downloaded onto an electronic device (e.g., devices 100, 300, and/or 500) and is ready to be launched (e.g., become opened) on the device. In some embodiments, a downloaded application becomes an installed application by way of an installation program that extracts program portions from a downloaded package and integrates the extracted portions with the operating system of the computer system.

As used herein, the terms “open application” or “executing application” refer to a software application with retained state information (e.g., as part of device/global internal state 157 and/or application internal state 192). An open or executing application is, optionally, any one of the following types of applications:
an active application, which is currently displayed on a display screen of the device that the application is being used on;
a background application (or background processes), which is not currently displayed, but one or more processes for the application are being processed by one or more processors; and
a suspended or hibernated application, which is not running, but has state information that is stored in memory (volatile and non-volatile, respectively) and that can be used to resume execution of the application.

As used herein, the term “closed application” refers to software applications without retained state information (e.g., state information for closed applications is not stored in a memory of the device). Accordingly, closing an application includes stopping and/or removing application processes for the application and removing state information for the application from the memory of the device. Generally, opening a second application while in a first application does not close the first application. When the second application is displayed and the first application ceases to be displayed, the first application becomes a background application.

In some embodiments, the computer system is in a locked state or an unlocked state. In the locked state, the computer system is powered on and operational but is prevented from performing a predefined set of operations in response to user input. The predefined set of operations optionally includes navigation between user interfaces, activation or deactivation of a predefined set of functions, and activation or deactivation of certain applications. The locked state can be used to prevent unintentional or unauthorized use of some functionality of the computer system or activation or deactivation of some functions on the computer system. In some embodiments, in the unlocked state, the computer system is powered on and operational and is not prevented from performing at least a portion of the predefined set of operations that cannot be performed while in the locked state. When the computer system is in the locked state, the computer system is said to be locked. When the computer system is in the unlocked state, the computer is said to be unlocked. In some embodiments, the computer system in the locked state optionally responds to a limited set of user inputs, including input that corresponds to an attempt to transition the computer system to the unlocked state or input that corresponds to powering the computer system off.

Attention is now directed towards embodiments of user interfaces (“UI”) and associated processes that are implemented on an electronic device, such as portable multifunction device 100, device 300, or device 500.

FIGS. 6A-6AC illustrate exemplary user interfaces for tracking and providing user health metrics, in accordance with some embodiments. The user interfaces in these figures are used to illustrate the processes described below, including the processes in FIG. 7, FIG. 8, and FIG. 9.

FIG. 6A illustrates computer system 600, which is a smart watch with touch-sensitive display 602 and rotatable and depressible input mechanism 604 (e.g., a rotatable and depressible crown). In various embodiments disclosed herein, computer system 600 measures, tracks, and/or presents various health metrics of the user of computer system 600. For example, in some embodiments, computer system 600 measures and/or tracks (e.g., using one or more internal and/or external sensors) the user's heart rate, respiratory rate, body temperature, blood oxygen level, and sleep duration. In some embodiments, computer system 600 and/or one or more other devices measure the user's health metrics while the user sleeps. In FIGS. 6A and 6B, computer system 600 displays user interface 608. In the depicted embodiments, user interface 608 is a daily metrics user interface that displays health metrics for the user for a particular day (e.g., a particular 24-hour period or for a particular calendar day). In some embodiments in which the user's health metrics are measured while the user is sleeping, user interface 608 displays the health metrics that were measured for the user the previous night. For example, in FIG. 6B, user interface 608 indicates that it is displaying health metrics for “TODAY” (e.g., selected day indication 608c) which, in some embodiments, means that user interface 608 is displaying health metrics that were recorded for the user the previous night while the user was sleeping.

User interface 608 includes option 608a, platter 608b, selected day indication 608c, and chart 610. Option 608a, when selected, causes computer system 600 to switch from displaying user interface 608a (e.g., a daily metrics user interface) to displaying a different health metrics user interface (e.g., a weekly health metrics user interface), as will be described in greater detail below. Selected day indication 608c indicates the day for which health data is being displayed in user interface 608. For example, in FIGS. 6A-6B, selected day indication 608c indicates that health metrics are being displayed for “TODAY.” As will be described in greater detail below, the user of computer system 600 is able to select different days to view health metrics for those different days, and selected day indication 608c is updated accordingly.

Chart 610 displays health metric information for the selected day. In the depicted embodiments, chart 610 displays an indication of the user's measured heart rate (e.g., in the area of chart 610 directly above the heart icon that is the leftmost icon in region 610d); respiratory rate (e.g., in the area of chart 610 directly above the lungs icon that is the second icon from the left in region 610d); body temperature (e.g., in the area of chart 610 directly above the thermometer icon that is the middle icon in region 610d); bloody oxygen level (e.g., in the area of chart 610 directly above the drop icon that is the second icon from the right in region 610d); and sleep duration (e.g., in the area of chart 610 directly above the bed icon that is the rightmost icon in region 610d). In FIG. 6A, chart 610 does not display health metric information, while in FIG. 6B, chart 610 does display health metric information. In some embodiments, computer system 600 measures health metrics for the user over a period of time and determines an average and/or typical range for the user for each metrics, e.g., a baseline range for each metric. In some embodiments, computer system 600 collects data for a threshold number of days (e.g., 5 days, 7 days, 10 days, 14 days) in order to determine the baseline range for the user for each of the health metrics. In FIG. 6A, computer system 600 does not yet have enough data to generate a baseline range for the health metrics, and displays an indication within platter 608b that computer system 600 must collect health metrics for seven more nights in order to generate the baseline range for the user.

At FIG. 6B, computer system 600 has collected data for the required threshold duration of time and/or the threshold number of days, and now displays the user's health metrics for the current day. In FIG. 6B, computer system 600 displays, within chart 610: object 612a, which is representative of the user's measured heart rate for the selected day; object 612b, which is representative of the user's measured respiratory rate for the selected day; object 612c, which is representative of the user's measured body temperature for the selected day; object 612d, which is representative of the user's measured blood oxygen level for the selected day; and object 612e, which is representative of the user's sleep duration for the selected day. Chart 610 includes three different regions, 610a, 610b, 610c. Region 610a is representative of the user's baseline range, such that an object displayed within region 610a indicates that the measured health metric is within the user's calculated baseline range. Region 610b is representative of a health metric being above the user's baseline range, such that an object displayed within region 610b indicates that the health metric corresponding to the object was measured and/or determined to be above the user's typical baseline range. Region 610c is representative of a health metric being below the user's baseline range, such that an object displayed within region 610c indicates that the health metric corresponding to the object was measured and/or determined to be below the user's typical baseline range.

While the various health metrics have different absolute values (e.g., body temperature can range from 90 degrees to 110 degrees while sleep duration can range from 0 hours to 24 hours), in some embodiments the different health metrics are charted on the same chart 610 by charting them based on spread from a median or average value. For example, in some embodiments, the center line of region 610a between the upper boundary and lower boundary of region 610a represents a median value for each health metric, such that an object displayed at the very center line indicates that the measured health metric is exactly at the user's median value or mean value for that metric. In some embodiments, the upper boundary of region 610a represents the third quartile+1.5 interquartile range (IQR) (e.g., Q3+1.5IQR), and the lower boundary of region 610a represents the first quartile−1.5 IQR (e.g., Q1−1.5 IQR). In such embodiments, objects displayed in the upper half of region 610a represent a value that is between the median and Q3+1.5IQR, while objects displayed in the lower half of region 610a represent a value that is between the median and Q1−1.5IQR. Objects displayed in region 610b are outliers that are above Q3+1.5IQR, and objects displayed in region 610c are outliers that are below Q1−1.5IQR. Accordingly, in some embodiments, a measurement for a respective health metric is determined to be outside of the user's baseline range if the measurement is determined to be above Q3+1.5IQR or below Q1−1.5IQR for the respective health metric. In FIG. 6B, all five health metrics (heart rate, respiratory rate, body temperature, blood oxygen level, and sleep duration) are within the user's baseline range. Object 612a, representative of the user's heart rate, is positioned in the lower half of region 610a, indicating that the user's measured heart rate for the selected day was below the user's median heart rate value, but above Q1−1.5IQR for heart rate. Object 612b, representative of the user's respiratory rate, is positioned in the center of region 610a, indicating that the user's measured respiratory rate for the selected day was at the user's median value for respiratory rate. Object 612c, representative of the user's body temperature, is positioned in the lower half of region 610a, indicating that the user's measured body temperature was below the user's median body temperature, but above Q1−1.5IQR for body temperature. Object 612d, representative of the user's blood oxygen level, is in the top half of region 610a, indicating that the user's measured blood oxygen level for the selected day was above the user's median blood oxygen level, but below Q3+1.5IQR for blood oxygen level. Object 612e, representative of the user's sleep duration, is in the top half of region 610a, indicating that the user's measured sleep duration for the selected day was above the user's median sleep duration, but below Q3+1.5IQR for sleep duration. In FIG. 6B, based on a determination that all five of the health metrics are within the baseline range for the selected day, platter 608b indicates that the user's overnight vitals are “typical” (e.g., within the baseline range). Additionally, platter 608b displays the time range during which the user's health metrics were collected on the selected day (e.g., 10:08 PM-6:05 AM). In some embodiments, the user's baseline range is adjusted over time as more data is collected. In some embodiments, the user's baseline range is calculated using a weighted average and/or a moving weighted average (e.g., over 30 days, 60 days, 90 days, 120 days, 240 days, or 365 days). In some embodiments, the weighted average gives greater weight to more recent days and measurements.

At FIG. 6C, computer system 600 displays time user interface 614. Time user interface 614 is a watch face that includes indication of time 614a, which includes hour hand 614a-1 and minute hand 614a-2. Time user interface 614 also includes complications 616a-616h, which correspond to different applications. For example, complication 616a corresponds to a calendar application and, when selected, causes computer system 600 to open the calendar application; complication 616b corresponds to a music application and, when selected, causes computer system 600 to open a music application; complication 616c corresponds to a weather application and, when selected, causes computer system 600 to open the weather application; and complication 616d corresponds to a fitness application and, when selected, causes computer system 600 to open the fitness application. At FIG. 6C, computer system 600 determines that the user's measured health metrics from the previous night include one or more health metrics that are outside of the user's baseline range.

At FIG. 6D, in response to the determination that the user's measured health metrics from the previous night include one or more health metrics that are outside of the user's baseline range, computer system 600 displays notification 618. Notification 618 indicates that during sleep, the user's body temperature, heart rate, and bloody oxygen levels were outside of their baseline ranges. Notification 618 further includes chart 610. In chart 610, object 612a, representative of the user's heart rate, and object 612c, representative of the user's body temperature, are displayed in region 610b, indicating that those two health metrics were measured to be above the user's baseline range. Object 612d, representative of the user's blood oxygen level, is displayed in region 610c, indicating that that health metric was measured below the user's baseline range.

In FIG. 6D, computer system 600 has detected that the user recently traveled to a higher elevation. Based on this determination, computer system 600 displays, within notification 618, information 618d, which indicates that the user recently traveled to a higher elevation. In some embodiments, when computer system 600 displays notification 618 indicating that one or more of the user's health metrics are outside of baseline, computer system 600 also determines whether one or more potential health factors are relevant to the user (e.g., pregnancy, travel, change in elevation, and/or lack of sleep), and if potential relevant health factors are present, the potential relevant health factors are displayed in notification 618.

User interface 618 includes options 618b-618c. Option 618b, when selected, causes computer system 600 to display user interface 608. Option 618c, when selected, causes computer system 600 to cease display of notification 618 and return to a previously-displayed user interface (e.g., time user interface 614 in FIGS. 6C-6D). At FIG. 6D, computer system 600 detects user input 619, which is a touch input (e.g., a tap input) corresponding to selection of option 618b.

At FIG. 6E, in response to user input 619, computer system 600 displays user interface 608. As discussed above, user interface 608 now shows that the user's heart rate, body temperature, and blood oxygen level (represented by objects 612a, 612c, and 612d, respectively) are outside of the user's baseline range. Based on a determination that there are three health metrics that are outside of the user's baseline range, platter 608b now shows that there are “3 outliers.” At FIG. 6E, computer system 600 detects user input 620, which is a touch input (e.g., a tap input) corresponding to selection of option 608a.

At FIG. 6F, in response to user input 620, computer system 600 displays user interface 622. As discussed above, in some embodiments, user interface 608 is a daily metrics user interface that displays health metrics for a particular day. In some embodiments, user interface 622 is a weekly metrics user interface that displays health metrics for a particular week (e.g., a particular 7-day period and/or a particular calendar week). User interface includes option 622a that, when selected, causes computer system to display user interface 608. User interface 622 also includes platter 622b, and chart 624. Like chart 610, chart 624 is split into three regions, 624a, 624b, 624c. Region 624a (like region 610a) represents the baseline range calculated for the user (e.g., from Q1−1.5IQR to Q3+1.5IQR), region 624b (like region 610b) represents being above the baseline range, and region 624c (like region 610c) represents being below the baseline range. While different vertical slices of chart 610 represented different health metrics, in user interface 622, different vertical slices of chart 624 represent different days of the week (e.g., Wednesday, Thursday, Friday, Saturday, Sunday, Monday, and Tuesday in FIG. 6F). In FIG. 6F, object 626a represents the user's health metrics measured for Wednesday. There are no objects in region 624b or region 624c above the “W” that represents Wednesday, indicating that none of the user's health metrics were outside of the baseline range on Wednesday. Object 626b is displayed above the “T” representing Thursday, and represents the user's health metrics measured for Thursday. Object 626c is displayed above the “F” representing Friday, and represents the user's health metrics measured for Friday. Object 626d is displayed above the “S” representing Saturday, and represents the user's health metrics measured for Saturday. Object 626e is displayed above the “S” representing Sunday, and represents the user's health metrics measured for Sunday. Object 626f is displayed above the “M” representing Monday, and represents the user's health metrics measured for Monday. The regions for Wednesday, Thursday, Friday, Saturday, Sunday, and Monday do not include any objects in region 624b or region 624c, indicating that the user's health metrics from Wednesday through Monday did not include any outliers that fell outside the baseline range for the user. However, the region above the “T” for Tuesday includes object 626g in region 624a, indicating that one or more health metrics were within the baseline range, as well as objects 626g-1 in region 624b, indicating that one or more health metrics were above the baseline range, and object 626g-2 in region 624c, indicating that one or more health metrics were below the baseline range.

In some embodiments, the size and/or placement of objects in region 624a depends on the measured values for the various health metrics for a particular day. For example, in some embodiments, object 626d is representative of all metrics for Tuesday (e.g., the current day) that fell within the baseline range for the user, which includes the user's respiratory rate and the user's seep duration, as seen in FIG. 6E. In some embodiments, the size and/or placement of object 626d is determined based on the measurements of the in-baseline health metrics for Tuesday, and where they would be displayed within region 610a in user interface 608. For example, in FIG. 6E, the lowest object in region 610a is object 612b and, accordingly, the lower end of object 626d in FIG. 6F is positioned at the same vertical position as object 612b; and in FIG. 6E, the lowest object in region 610a is object 612e and, accordingly, the upper end of object 626g in FIG. 6F is positioned at the same vertical position as object 612e.

At FIG. 6F, computer system 600 detects user input 627, which is a touch input (e.g., a tap input) corresponding to selection of option 622a. At FIG. 6G, in response to user input 627, computer system 600 re-displays user interface 608. At FIG. 6G, computer system 600 detects user input 628, which is a rotational input of rotatable and depressible input mechanism 604.

FIGS. 6H1 through 6H5 show how a user can scroll through the different health metrics and view more specific information for each health metric via a rotational input with rotatable and depressible input mechanism 604. At FIG. 6H1, in response to user input 628, computer system 600 displays object 612a in a darker color to indicate that the heart rate metric is selected. In some embodiments, rotation of rotatable and depressible input mechanism 604 in a first rotation direction (e.g., clockwise) will cause the selection indication to move in a first direction (e.g., to the right) and rotation in a second rotation direction (e.g., counterclockwise) will cause the selection indication to move in a second direction different from the first direction (e.g., to the left). Additionally, in response to user input 628, computer system 600 displays heart rate information in platter 608b. The heart rate information in platter 608b indicates that the user's measured heart rate the previous night was 116 beats per minute, and that this value was 25 beats per minute above the user's average heart rate. At FIG. 6H1, computer system 600 detects user input 629b, which is a rotation of rotatable and depressible input mechanism 604.

At FIG. 6H2, in response to user input 629b, computer system 600 displays object 612b in a darker color to indicate that the respiratory rate metric is selected. Additionally, in response to user input 629b, computer system 600 displays respiratory rate information in platter 608b. The respiratory rate information in platter 608b indicates that the user's measured respiratory rate the previous night was 12.2 breaths per minute, and that this value is within the user's typical (e.g., baseline) range. At FIG. 6H2, computer system 600 detects user input 629d, which is a rotation of rotatable and depressible input mechanism 604.

At FIG. 6H3, in response to user input 629d, computer system 600 displays object 612c in a darker color to indicate that the body temperature metric is selected. Additionally, in response to user input 629d, computer system 600 displays body temperature information in platter 608b. The body temperature information in platter 608b indicates that the user's measured body temperature the previous night was 101 degrees, and that this value is above the user's typical (e.g., baseline) range. At FIG. 6H3, computer system 600 detects user input 629f, which is a rotation of rotatable and depressible input mechanism 604.

At FIG. 6H4, in response to user input 629f, computer system 600 displays object 612d in a darker color to indicate that the blood oxygen level metric is selected. Additionally, in response to user input 629f, computer system 600 displays blood oxygen level information in platter 608b. The blood oxygen level information in platter 608b indicates that the user's measured blood oxygen level the previous night was 92%, and that this value is lower than the user's typical (e.g., baseline) range. At FIG. 6H4, computer system 600 detects user input 629h, which is a rotation of rotatable and depressible input mechanism 604.

At FIG. 6H5, in response to user input 629h, computer system 600 displays object 612e in a darker color to indicate that the sleep duration metric is selected. Additionally, in response to user input 629h, computer system 600 displays sleep duration information in platter 608b. The sleep duration information in platter 608b indicates that the user's measured sleep duration the previous night was 8 hours and 4 minutes, and that this value is within the user's typical (e.g., baseline) range. At FIG. 6H4, computer system 600 detects user input 629j, which is a rotation of rotatable and depressible input mechanism 604. In some embodiments, in response to user input 629, computer system 600 returns to the state shown in FIG. 6G, in which none of the individual health metrics is selected.

FIGS. 6H1 through 6H5 in combination with FIGS. 6I through 6M also demonstrate a feature in which, while a particular health metric is selected, a user is able to select option 608a to view a weekly metrics user interface (e.g., user interface 622) specifically for the selected health metric.

At FIG. 6H1, while the heart rate metric is selected, computer system 600 detects user input 629a, which is a touch input (e.g., a tap input) corresponding to selection of option 608a. At FIG. 61, in response to user input 629a, computer system 600 displays user interface 622, which was described above with reference to FIG. 6F. However, in FIG. 6I, rather than objects 626a-626g representing all of the user's health metrics on each day, objects 626a-626g now represent the user's heart rate metric on each day. For example, in FIG. 6I, object 626a is indicative of the user's measured heart rate metric for Wednesday, object 626b is indicative of the user's measured heart rate metric for Thursday, object 626c is indicative of the user's measured heart rate metric for Friday, and so forth. Additionally, whereas in FIG. 6F, platter 622b showed how many outliers there were in a given day (e.g., for a selected day), platter 622b now shows the user's heart rate metric for the selected day.

At FIG. 6H2, while the respiratory rate metric is selected, computer system 600 detects user input 629c, which is a touch input (e.g., a tap input) corresponding to selection of option 608a. At FIG. 6J, in response to user input 629c, computer system 600 displays user interface 622, but objects 626a-626g now represent the user's respiratory rate metric on each day. For example, in FIG. 6J, object 626a is indicative of the user's measured respiratory rate metric for Wednesday, object 626b is indicative of the user's measured respiratory rate metric for Thursday, object 626c is indicative of the user's measured respiratory rate metric for Friday, and so forth. Additionally, platter 622b now shows the user's respiratory rate metric for the selected day.

At FIG. 6H3, while the body temperature metric is selected, computer system 600 detects user input 629e, which is a touch input (e.g., a tap input) corresponding to selection of option 608a. At FIG. 6K, in response to user input 629e, computer system 600 displays user interface 622, but objects 626a-626g now represent the user's body temperature metric on each day. For example, in FIG. 6K, object 626a is indicative of the user's measured body temperature metric for Wednesday, object 626b is indicative of the user's measured body temperature metric for Thursday, object 626c is indicative of the user's measured body temperature metric for Friday, and so forth. Additionally, platter 622b now shows the user's body temperature metric for the selected day.

At FIG. 6H4, while the blood oxygen level metric is selected, computer system 600 detects user input 629g, which is a touch input (e.g., a tap input) corresponding to selection of option 608a. At FIG. 6L, in response to user input 629g, computer system 600 displays user interface 622, but objects 626a-626g now represent the user's blood oxygen level metric on each day. For example, in FIG. 6L, object 626a is indicative of the user's measured blood oxygen level metric for Wednesday, object 626b is indicative of the user's measured blood oxygen level metric for Thursday, object 626c is indicative of the user's measured blood oxygen level metric for Friday, and so forth. Additionally, platter 622b now shows the user's blood oxygen level metric for the selected day.

At FIG. 6H5, while the sleep duration metric is selected, computer system 600 detects user input 629i, which is a touch input (e.g., a tap input) corresponding to selection of option 608a. At FIG. 6M, in response to user input 629i, computer system 600 displays user interface 622, but objects 626a-626g now represent the user's sleep duration metric on each day. For example, in FIG. 6M, object 626a is indicative of the user's measured sleep duration metric for Wednesday, object 626b is indicative of the user's measured sleep duration metric for Thursday, object 626c is indicative of the user's measured sleep duration metric for Friday, and so forth. Additionally, platter 622b now shows the user's sleep duration metric for the selected day.

FIGS. 6M through 6N6 demonstrate a feature in which a user can scroll through and/or select different days within user interface 622 via user input, such as rotation of rotatable and depressible input mechanism 604. In FIG. 6M, the current day (Tuesday), represented by object 626g, is selected, as indicated by object 626g being displayed in a darker color than objects 626a-626f. At FIG. 6M, computer system 600 detects user input 630, which is a rotation of rotatable and depressible input mechanism 604.

At FIG. 6N1, in response to user input 630, computer system 600 displays object 626f in a darker color to indicate that Monday is now selected, and displays Monday's sleep duration information within platter 622b. In some embodiments, rotation of rotatable and depressible input mechanism 604 in a first rotation direction (e.g., clockwise) will cause the selection indication to move in a first direction (e.g., to the right) and rotation in a second rotation direction (e.g., counterclockwise) will cause the selection indication to move in a second direction different from the first direction (e.g., to the left). At FIG. 6N1, computer system 600 detects user input 632a, which is a rotation of rotatable and depressible input mechanism 604.

At FIG. 6N2, in response to user input 632a, computer system 600 displays object 626e in a darker color to indicate that Sunday is now selected, and displays Sunday's sleep duration information within platter 622b. At FIG. 6N2, computer system 600 detects user input 632b, which is a rotation of rotatable and depressible input mechanism 604.

At FIG. 6N3, in response to user input 632b, computer system 600 displays object 626d in a darker color to indicate that Saturday is now selected, and displays Saturday's sleep duration information within platter 622b. At FIG. 6N3, computer system 600 detects user input 632c, which is a rotation of rotatable and depressible input mechanism 604.

At FIG. 6N4, in response to user input 632c, computer system 600 displays object 626c in a darker color to indicate that Friday is now selected, and displays Friday's sleep duration information within platter 622b. At FIG. 6N4, computer system 600 detects user input 632d, which is a rotation of rotatable and depressible input mechanism 604.

At FIG. 6N5, in response to user input 632d, computer system 600 displays object 626b in a darker color to indicate that Thursday is now selected, and displays Thursday's sleep duration information within platter 622b. At FIG. 6N5, computer system 600 detects user input 632e, which is a rotation of rotatable and depressible input mechanism 604.

At FIG. 6N6, in response to user input 632c, computer system 600 displays object 626a in a darker color to indicate that Wednesday is now selected, and displays Wednesday's sleep duration information within platter 622b. At FIG. 6N6, computer system 600 detects user input 632f, which is a rotation of rotatable and depressible input mechanism 604. In some embodiments, in response to user input 632f, computer system 600 returns to the state shown in FIG. 6M.

FIG. 6O depicts an example week within user interface 622. In FIG. 6O, user interface 622 is displayed without any particular health metric selected, such that chart 624 shows a summary of all of the health metrics, rather than charting a single health metric (as was the case in FIGS. 6I through 6N6). In FIG. 6O, all five health metrics were within the baseline range on Wednesday, as represented by object 626a and as indicated by the lack of any objects in region 624b and region 624c for Wednesday. On Thursday, one or more health metrics were within the baseline range, as represented by object 626b in region 624a, and one or more health metrics were above the baseline range, as represented by object 626b-1 in region 624b. On Thursday, no health metrics were below the baseline range, as indicated by the lack of any objects in region 624b for Thursday. On Friday, one or more health metrics were within the baseline range, as represented by object 626c in region 624a, and one or more health metrics were below the baseline range, as represented by object 626c-2 in region 624c. On Friday, no health metrics were above the baseline range, as indicated by the lack of any objects in region 624a for Friday. On Saturday, one or more health metrics were within the baseline range, as indicated by object 626d in region 624a; one or more health metrics were above the baseline range, as indicated by object 626d-1 in region 624b; and one or more health metrics were below the baseline range, as indicated by object 626d-2 in region 624c. On Sunday, Monday, and Tuesday, all health metrics were within the baseline range, as indicated by objects 626e, 626f, 626g in region 624a, respectively, and the lack of objects in regions 624b and 624c for Sunday, Monday, and Tuesday. At FIG. 6O, computer system 600 detects user input 633, which is a rotation of rotatable and depressible input mechanism 604.

At FIG. 6P, in response to user input 633, computer system 600 displays objects 626d, 626d-1, and 626d-2 in darker colors to indicate that Saturday is now selected. Additionally, in response to user input 633, computer system 600 updates platter 622b to indicate that there were two health metric outliers (e.g., two health metrics that were outside of baseline range) on Saturday, the selected day, and also to indicate that health metrics were recorded between 10:08 PM and 6:05 AM for Saturday (e.g., 10:08 PM on Friday to 6:08 AM on Saturday). FIG. 6P depicts two example scenarios in which computer system 600 detects two user inputs: user input 634a, corresponding to selection of option 622a, and user input 634b, corresponding to selection of option 622b-1.

At FIG. 6Q, in response to user input 634a, computer system 600 displays user interface 608 (e.g., the daily metrics user interface). Based on user input 634a being received while Saturday was selected, user interface 608 displays health metric information corresponding to the selected day, Saturday. It can be seen in user interface 608 that on Saturday, the user's respiratory rate was below the baseline range, and the user's blood oxygen level was above the user's baseline range.

At FIG. 6R, in response to user input 634b, computer system 600 displays user interface 636. User interface 636 includes option 636a that, when selected, causes computer system 600 to return to the state shown in FIG. 6P. User interface 636 also identifies the two health metrics that were outliers on Saturday, e.g., bloody oxygen level and respiratory rate.

At FIG. 6S, computer system 600 displays time user interface 638, which is a watch face user interface that includes time indication 638a as well as complications 638b-638d. Complication 638b corresponds to a fitness application and, in some embodiments, displays information provided by the fitness application and, when selected, causes computer system 600 to open the fitness application. Complication 638c corresponds to a stopwatch application and, in some embodiments, displays information provided by the stopwatch application and, when selected, causes computer system 600 to open the stopwatch application. Complication 638d corresponds to a health metrics application (e.g., a health metrics application that displays user interface 608 and/or user interface 622). In some embodiments, complication 638d displays chart 610 and/or portions of user interface 608. At FIG. 6T, time user interface 638 includes complication 638e instead of complication 638d. Complication 638e corresponds to the health metrics application, and displays chart 624 and/or portions of user interface 622.

FIGS. 6U through 6AC depict example scenarios in which health metric information is displayed on a different computer system, e.g., computer system 640. FIG. 6U depicts computer system 640, which is a smart phone with touch-sensitive display 642 and buttons 644a-644c. At FIG. 6U, computer system 640 displays health summary user interface 642. Health summary user interface 642 includes platters 646a-646f. Platter 646a displays an indication of a user's health metrics for the current day, as well as an indication of whether the user's health metrics are within the user's baseline ranges. In FIG. 6U, all of the user's health metrics are within the user's baseline ranges. In the depicted embodiment, platter 646a also displays a representation of user interface 608 and/or chart 610 (or an analogous user interface and/or chart displayed on computer system 640) for the current day. Platter 646b displays user heart rate information for the current day and, in some embodiments, when selected, causes computer system 600 to display a heart rate user interface with more detailed heart rate information (e.g., historical heart rate information). Platter 646c displays user body temperature information for the current day and, in some embodiments, when selected, causes computer system 600 to display a body temperature user interface with more detailed body temperature information (e.g., historical body temperature information). Platter 646d displays calorie information for the current day (e.g., how many calories the user has burned based on detected user activity levels) and, in some embodiments, when selected, causes computer system 600 to display a calorie user interface with more detailed calorie information (e.g., historical calories burned information). Platter 646e displays steps information for the current day (e.g., how many steps the user has taken in the current day) and, in some embodiments, when selected, causes computer system 600 to display a steps interface with more detailed steps information (e.g., historical steps taken information). Platter 646f, when selected, causes computer system 600 to display additional health data that is not displayed in health summary user interface 642. At FIG. 6U, computer system 640 detects user input 647, which is a touch input (e.g., a tap input) corresponding to selection of platter 647.

At FIG. 6V, in response to user input 647, computer system 640 displays user interface 648. User interface 648 is a daily metrics user interface similar to user interface 608 discussed above. User interface 648 includes options 648a-648d, chart 650, and platter 648c. Option 648a, when selected, causes computer system 640 to display user interface 648. Option 648b, when selected, causes computer system 640 to display a weekly metrics user interface, as will be described in greater detail below. Option 648c, when selected, causes computer system 640 to display a monthly metrics user interface, as will be described in greater detail below. Option 648d, when selected, causes computer system 640 to display a six-month metrics user interface, as will be described in greater detail below. Chart 650 includes many and/or all of the features of chart 610 described above. Chart 650 includes regions 650a-650c. Region 650a is representative of metrics being within the user's baseline range (e.g., like region 610a described above), region 650b is representative of metrics being above the user's baseline range (e.g., like region 610b described above), and region 650c is representative of metrics being below the user's baseline range (e.g., like region 610c described above). Chart 650 includes objects 652a-652e, which correspond to and/or are representative of different respective health metrics (e.g., heart rate, respiratory rate, body temperature, bloody oxygen level, and sleep duration, respectively). In FIG. 6V, platter 648e indicates that all of the user's health metrics are within the user's baseline range. User interface 648 also includes option 648f which, when selected, causes computer system 600 to display a user interface that displays all of the individual sensor readings and/or measurements that were made the previous night. At FIG. 6V, computer system 640 detects user input 653, which is a touch input (e.g., a tap input) corresponding to selection of object 652d, which is representative of the blood oxygen level metric.

At FIG. 6W, in response to user input 653, computer system 640 displays lollipop 654, which displays the user's blood oxygen level measurement for the displayed and/or selected day (which, in FIG. 6W, is the current day). In this way, a user is able to select an individual object 652a-652e to view the specific measurement for the health metric that corresponds to the selected object. At FIG. 6W, computer system 640 detects user input 655, which is a touch input (e.g., a tap input) corresponding to selection of option 648b.

At FIG. 6X, in response to user input 655, computer system 650 displays user interface 656, which is a weekly metrics user interface similar to user interface 622 discussed above. User interface 656 includes options 648a-648d, chart 658, and platter 656a. Chart 658 includes regions 658a, 658b, 658c. Region 658a is representative of metrics being within the user's baseline range (e.g., like region 624a described above), region 658b is representative of metrics being above the user's baseline range (e.g., like region 624b described above), and region 658c is representative of metrics being below the user's baseline range (e.g., like region 624c described above). Chart 658 includes objects 660a-66g. Object 660a is representative of the user's health metrics on Sunday; object 660b is representative of the user's health metrics on Monday; object 660c is representative of the user's health metrics on Tuesday; object 660d is representative of the user's health metrics on Wednesday; object 660e is representative of the user's health metrics on Thursday; object 660f is representative of the user's health metrics on Friday; and object 660g is representative of the user's health metrics on Saturday. Object 660a, 660b, 660d, 660f, and 660g each are positioned entirely within region 658a, indicating that all of the user's health metrics were within the baseline range on Sunday, Monday, Wednesday, Friday, and Saturday, respectively. Object 660c extends from region 658a to region 658c, indicating that one or more health metrics were within baseline range on Tuesday, and one or more health metrics were below the baseline range on Tuesday. Object 660e is positioned in all three regions, 658a, 658b, 658c, indicating that one or more health metrics were above the baseline range, one or more health metrics were within the baseline range, and one or more health metrics were below the baseline range on Thursday. In some embodiments, the object representative of a particular day includes one or more sub-objects that are each representative of a single health metric. For example, object 660e includes one circle at the top of object 660e in region 658b, two circles in region 658a, and two circles in region 658c, indicating that one health metric was above baseline range, two health metrics were within baseline range, and two health metrics were below baseline range on Thursday. At FIG. 6X, computer system 640 detects user input 661, which is a touch input (e.g., a tap input) corresponding to selection of object 660c.

At FIG. 6Y, in response to user input 661, computer system 640 displays lollipop 662, which indicates that there are three outliers on the selected day (e.g., Thursday Nov. 10, 2023). Additionally, in response to user input 661, user interface 656 now displays individual health metric measurements for the selected day. In FIG. 6Y, the three outlier measurements (e.g., body temperature, heart rate, and sleep duration) are shown. However, in some embodiments, measurements for all health metrics are shown. At FIG. 6Y, computer system 640 detects user input 663, which is a touch input (e.g., a tap input) corresponding to selection of option 648c.

At FIG. 6Z, in response to user input 663, computer system 640 displays user interface 664, which is a monthly metrics user interface. Monthly metrics user interface 664 includes chart 666, which includes regions 666a-666c. As with the other charts discussed above, region 666a is representative of metrics within the user's baseline range, region 666b is representative of metrics above the user's baseline range, and region 666c is representative of metrics below the user's baseline range. Chart 666 includes objects 668 that show whether the user's health metrics were within or outside of baseline on a given day of the selected month. In some embodiments, each object of objects 668 is representative of one day in the month. At FIG. 6Z, computer system 640 detects user input 669, which is a touch input (e.g., a tap input) corresponding to selection of option 648d.

At FIG. 6AA, in response to user input 669, computer system 640 displays user interface 670, which is a six month metrics user interface that shows the user's health metrics over a six month period. User interface 670 includes chart 672, which includes regions 672-672c. As with the other charts discussed above, region 672a is representative of metrics within the user's baseline range, region 672b is representative of metrics above the user's baseline range, and region 672c is representative of metrics below the user's baseline range. Chart 672 includes objects 674 that show whether the user's health metrics were within or outside of baseline during the six month period. In some embodiments, each object of objects 674 is representative of one day in the six month period. In some embodiments, each object of objects 674 is representative of one week in the six month period.

As discussed above with reference to FIGS. 6S-6T, in various embodiments, the health metric information shown in the user interfaces above can also be displayed as widgets and/or complications in other user interfaces. In FIG. 6AB, computer system 640 displays lock screen transition user interface 676. Lock screen transition user interface 676 is indicative of computer system 640 having transitioned from a locked state to an unlocked state (e.g., based on receiving authentication information from the user). In some embodiments, lock screen transition user interface 676 is a lock screen user interface indicative of computer system 640 being in a locked state. Lock screen transition user interface 676 includes complication 678a, complication 678b, complication 678c (in the left figure), and complication 678d (in the right figure). Complication 678a corresponds to a fitness application, and displays information from the fitness application and, in some embodiments, when selected, causes computer system 640 to open the fitness application. Complication 678b corresponds to a health metrics application (e.g., a health metrics application that displays user interfaces 646, 648, 656, 664, and/or 670 described above), and indicates how many of the user's health metrics are outside of baseline range on the current day. For example, in FIG. 6AB, complication 678b includes first region 679a that is representative of metrics that are within the user's baseline range, region 679b that is representative of metrics that are above the user's baseline range, and region 679c that is representative of metrics that are below the user's baseline range. In FIG. 6AB, region 679a includes three circles that are representative of three health metrics that are within the user's baseline range for the current day; region 679b includes one circle that is representative of one health metric that is above the user's baseline range for the current day, and region 679c includes one circle that is representative of one health metric that is below the user's baseline range for the current day. In some embodiments, complication 678b, when selected, causes computer system 640 to display user interface 648.

Complication 678c corresponds to the health metrics application, and displays a representation of chart 650 and/or user interface 648 and, when selected, causes computer system 640 to display user interface 648. Complication 678d corresponds to the health metrics application, and displays a representation of chart 658 and/or user interface 656 and, when selected, causes computer system 640 to display user interface 656.

At FIG. 6AC, computer system 640 displays home screen user interface 680. Home screen user interface 680 includes application icons 682a-682m. Each application icon 682a-682m, when selected, causes computer system 640 to open a corresponding application. Home screen user interface also includes widget 684a (on the left figure) and widget 684b (on the right figure). Widget 684a corresponds to the health metrics application, and displays a representation of chart 650 and/or user interface 648 and, when selected, causes computer system 640 to display user interface 648. Widget 684b corresponds to the health metrics application, and displays a representation of chart 658 and/or user interface 656 and, when selected, causes computer system 640 to display user interface 656.

FIG. 7 is a flow diagram illustrating a method for tracking and providing user health metrics using a computer system in accordance with some embodiments. Method 700 is performed at a computer system (e.g., 100, 300, 500, 600, and/or 640) (e.g., a smart phone, a smart watch, a tablet, a laptop, a desktop, a wearable device, wrist-worn device, and/or head-mounted device) that is in communication with one or more display generation components (e.g., a display, a touch-sensitive display, and/or a display controller) (e.g., 602 and/or 642) and one or more input devices (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor) (e.g., 602, 604, 642, and/or 644a-c). Some operations in method 700 are, optionally, combined, the orders of some operations are, optionally, changed, and some operations are, optionally, omitted.

As described below, method 700 provides an intuitive way for tracking and providing user health metrics. The method reduces the cognitive burden on a user for tracking and accessing user health metrics, thereby creating a more efficient human-machine interface. For battery-operated computing devices, enabling a user to access health data faster and more efficiently conserves power and increases the time between battery charges.

The computer system (e.g., 600 and/or 640) displays (702), via the one or more display generation components (e.g., 602 and/or 6420), a daily metrics user interface (e.g., 608 and/or 648), including displaying, within the daily metrics user interface, first vital metric information that is representative of a first day (e.g., objects 612a-612e and/or objects 652a-652c) (e.g., a first day of the week, a first calendar day, and/or a first twenty-four hour period) (in some embodiments, the day user interface is representative of a first day without being representative of a second day and/or any other day) (e.g., first vital metric information that was captured during the first day and/or otherwise corresponds to the first day) (e.g., without displaying second vital metric information that is representative of a second day; without displaying vital metric information that corresponds to days other than the first day; without displaying vital metric information that does not correspond to the first day; and/or without displaying vital metric information that is not representative of the first day), wherein the first vital metric information includes two or more vital metrics (e.g., heart rate, respiratory rate, body temperature, blood-oxygen level, and/or sleep duration). While displaying the daily metrics user interface, the computer system detects (704), via the one or more input devices, a first user input (e.g., 629a, 629c, 629e, 629g, and/or 629i) (e.g., one or more user inputs, one or more touch inputs, one or more tap inputs, one or more hardware control inputs, one or more button presses, one or more rotational inputs, one or more gesture inputs, and/or one or more spoken inputs). In response to detecting the first user input (706): in accordance with a determination that a first vital metric of the two or more vital metrics was selected when the first user input was received (e.g., in each of FIGS. 6H1 through 6H5, a different vital metric is selected), the computer system displays (708), via the one or more display generation components, a first weekly metrics user interface (e.g., 622 in FIGS. 6I through 6M), wherein the first weekly metrics user interface includes first weekly vital metric information that is representative of a first week (e.g., a first calendar week and/or a first seven-day period) (in some embodiments, the first week includes the first day) and displays information pertaining to the first vital metric for the first week without displaying information corresponding to a second vital metric different from the first vital metric (e.g., in some embodiments, the first weekly metrics user interface displays information pertaining to the first vital metric over the course of the first week without displaying information pertaining to other vital metrics); and in accordance with a determination that the second vital metric different from the first vital metric was selected when the first user input was received (e.g., in each of FIGS. 6H1 through 6H5, a different vital metric is selected), the computer system displays (710), via the one or more display generation components, a second weekly metrics user interface (e.g., 622 in FIGS. 6I through 6M), wherein the second weekly metrics user interface includes second weekly vital metric information that is representative of the first week and displays information pertaining to the second vital metric for the first week without displaying information corresponding to first vital metric (e.g., in some embodiments, the second weekly metrics user interface displays information pertaining to the second vital metric over the course of the first week without displaying information pertaining to other vital metrics). Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, detecting the first user input comprises detecting, via the one or more input devices, a selection input corresponding to selection of a first displayed user interface object (e.g., 608a). Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, detecting the first user input comprises detecting, via the one or more input devices, a touch input (e.g., a tap input) (e.g., 629a, 629c, 629c, 629g, and/or 629i) corresponding to selection of the first displayed user interface object (e.g., 608a) (e.g., a touch input on a touch-sensitive display at a first position that corresponds to and/or is the display position of the first displayed user interface object). Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the daily metrics user interface (e.g., 608) in a first manner (e.g., with a first set of visual characteristics) indicating that the first vital metric of the two or more vital metrics is selected and the second vital metric of the two or more vital metrics is not selected, the computer system detects rotation of a rotatable input mechanism (e.g., 604) (e.g., a physical rotatable input mechanism and/or a physically rotatable input mechanism) (e.g., user inputs 628, 629b, 629d, 629f, and/or 629h). In response to detecting rotation of the rotatable input mechanism, the computer system displays the daily metrics user interface in a second manner indicating that the second vital metric is selected and the first vital metric is not selected (e.g., FIGS. 6G and 6H1 through 6H5 highlighting different objects as different metrics are selected). Allowing a user to select different vital metrics by rotating a rotatable input mechanism allows a user to perform these operations without displaying additional user interface objects, which conserves display space. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the daily metrics user interface (e.g. 608) includes: in accordance with a determination that the first vital metric is selected (e.g., and the second vital metric is not selected) (e.g., in each of FIGS. 6H1 through 6H5, a different metric is selected), displaying, within a first region (e.g., 608b) of the daily metrics user interface, a first measured value corresponding to the first vital metric for the first day (e.g., a numerical value and/or other measured value for the first vital metric measured on and/or for the first day) (e.g., without displaying a measured value corresponding to the second vital metric for the first day); and in accordance with a determination that the second vital metric is selected (e.g., and the first vital metric is not selected) (e.g., in each of FIGS. 6H1 through 6H5, a different metric is selected), displaying, within the first region (e.g., 608b) of the daily metrics user interface, a second measured value corresponding to the second vital metric for the first day (e.g., a numerical value and/or other measured value for the second vital metric measured on and/or for the first day) (e.g., without displaying a measured value corresponding to the first vital metric for the first day). Allowing a user to switch between different vital metrics via user input in order to see the measured value for each vital metric allows the user to access this information while conserving display space. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first weekly metrics user interface (e.g., 622) includes concurrently displaying: a representation of a first day (e.g., 626a-626g) of the first week that is indicative of a first measured value for the first vital metric for the first day of the first week (e.g., a numerical value and/or measured value that was measured for the first vital metric on and/or for the first day of the first week); a representation of a second day (e.g., 626a-626g) of the first week that is different from the representation of the first day of the first week and that is indicative of a second measured value for the first vital metric for the second day of the first week (e.g., a numerical value and/or measured value that was measured for the first vital metric on and/or for the second day of the first week); a representation of a third day (e.g., 626a-626g) of the first week that is different from the representation of the first day of the first week and the representation of the second day of the first week, and that is indicative of a third measured value for the first vital metric for the third day of the first week (e.g., a numerical value and/or measured value that was measured for the first vital metric on and/or for the third day of the first week); a representation of a fourth day (e.g., 626a-626g) of the first week that is different from the representation of the first day, the representation of the second day, and the representation of the third day, and that is indicative of a fourth measured value for the first vital metric for the fourth day of the first week (e.g., a numerical value and/or measured value that was measured for the first vital metric on and/or for the fourth day of the first week); and a representation of a fifth day (e.g., 626a-626g) of the first week that is different from the representation of the first day, the representation of the second day, the representation of the third day, and the representation of the fourth day, and that is indicative of a fifth measured value for the first vital metric for the first day of the first week (e.g., a numerical value and/or measured value that was measured for the first vital metric on and/or for the fifth day of the first week). Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first weekly metrics user interface includes: in accordance with a determination that the first day of the first week is selected (e.g., and the other days of the first week are not selected) (e.g., in each of FIGS. 6N1 through 6N6, a different day of the week is selected), displaying, within a first region (e.g., 622b in FIGS. 6N1 through 6N6) of the first weekly metrics user interface (e.g., 622), the first measured value corresponding to the first vital metric for the first day of the first week (e.g., without displaying a measured value corresponding to the other days of the first week); in accordance with a determination that the second day of the first week is selected (e.g., and the other days of the first week are not selected), displaying, within the first region (e.g., 622b in FIGS. 6N1 through 6N6) of the first weekly metrics user interface, the second measured value corresponding to the first vital metric for the second day of the first week (e.g., without displaying a measured value corresponding to the other days of the first week); in accordance with a determination that the third day of the first week is selected (e.g., and the other days of the first week are not selected), displaying, within the first region of the first weekly metrics user interface (e.g., 622b in FIGS. 6N1 through 6N6), the third measured value corresponding to the first vital metric for the third day of the first week (e.g., without displaying a measured value corresponding to the other days of the first week); in accordance with a determination that the fourth day of the first week is selected (e.g., and the other days of the first week are not selected), displaying, within the first region of the first weekly metrics user interface (e.g., 622b in FIGS. 6N1 through 6N6), the fourth measured value corresponding to the first vital metric for the fourth day of the first week (e.g., without displaying a measured value corresponding to the other days of the first week); and in accordance with a determination that the fifth day of the first week is selected (e.g., and the other days of the first week are not selected), displaying, within the first region of the first weekly metrics user interface (e.g., 622b in FIGS. 6N1 through 6N6), the fifth measured value corresponding to the first vital metric for the fifth day of the first week (e.g., without displaying a measured value corresponding to the other days of the first week). Allowing a user to switch between different days of the week via user input in order to see the measured value for the first vital metric during each day of the week allows the user to access this information while conserving display space. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first weekly metrics user interface (e.g., 622) in a first manner (e.g., with a first set of visual characteristics) indicating that the first day of the first week is selected (e.g., and other days of the first week are not selected) (e.g., FIG. 6N1), the computer system detects a rotation input (e.g., 632a, 632b, 632c, 632d, 632c, and/or 632f) that includes rotation of a rotatable input mechanism (e.g., 604) (e.g., a physical rotatable input mechanism and/or a physically rotatable input mechanism). In response to detecting the rotation input: in accordance with a determination that the rotation input comprises rotation of the rotatable input mechanism by a first amount, the computer system displays the first weekly metrics user interface in a second manner (e.g., with a second set of visual characteristics) that is different from the first manner and that indicates that the second day of the first week is selected (e.g., FIG. 6N2); in accordance with a determination that the rotation input comprises rotation of the rotatable input mechanism by a second amount different from the first amount, the computer system displays the first weekly metrics user interface in a third manner (e.g., with a third set of visual characteristics) that is different from the first manner and the second manner and that indicates that the third day of the first week is selected (e.g., FIG. 6N3); in accordance with a determination that the rotation input comprises rotation of the rotatable input mechanism by a third amount different from the first amount and the second amount, the computer system displays the first weekly metrics user interface in a fourth manner (e.g., with a fourth set of visual characteristics) that is different from the first manner, the second manner, and the third manner, and that indicates that the fourth day of the first week is selected (e.g., FIG. 6N4); and in accordance with a determination that the rotation input comprises rotation of the rotatable input mechanism by a fourth amount different from the first amount, the second amount, and the third amount, the computer system displays the first weekly metrics user interface in a fifth manner (e.g., with a fifth set of visual characteristics) that is different from the first manner, the second manner, the third manner, and the fourth manner, and that indicates that the fifth day of the first week is selected (e.g., FIG. 6N5). Allowing a user to switch between different days of the week via a rotation input in order to see the measured value for the first vital metric during each day of the week allows the user to access this information while conserving display space. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the daily metrics user interface (e.g., 608) includes concurrently displaying a representation of the first vital metric (e.g., 612a, 612b, 612c, 612d, and/or 612c) and a representation of the second vital metric (e.g., 612a, 612b, 612c, 612d, and/or 612c). In some embodiments, concurrently displaying the representation of the first vital metric and the representation of the second vital metric comprises: in accordance with a determination that the first vital metric falls within a threshold range for the first day, displaying the representation of the first vital metric in a first visual manner (e.g., with a first set of visual characteristics) (e.g., displaying the representation of the first vital metric within a first display region, at a first size, with a first shape, and/or with a first color) indicating that the first vital metric falls within the threshold range for the first day (e.g., displaying object 612a, 612b, 612c, 612d, and/or 612e within region 610a); and in accordance with a determination that the first vital metric falls outside the threshold range for the first day, displaying the representation of the first vital metric in a second visual manner (e.g., with a second set of visual characteristics) (e.g., displaying the representation of the first vital metric outside the first display region, at a second size, with a second shape, and/or with a second color) that is different from the first visual manner and that indicates that the first vital metric falls outside the threshold range for the first day (e.g., displaying object 612a, 612b, 612c, 612d, and/or 612e outside of region 610a and/or within region 610b or 610c). Displaying the representation of the first vital metric differently based on whether or not the first vital metric is within the threshold range provides the user with improved visual feedback about a state of the system (e.g., the system has determined that the first vital metric is inside or outside the threshold range). Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the representation of the first vital metric (e.g., 612a, 612b, 612c, 612d, and/or 612e) in the first visual manner comprises displaying the representation of the first vital metric within a first display region (e.g., 610a) of the daily metrics user interface that is indicative of the first vital metric falling within the threshold range for the first day; and displaying the representation of the first vital metric in the second visual manner comprises displaying the representation of the first vital metric outside of the first display region (e.g., 610a) of the daily metrics user interface. Displaying the representation of the first vital metric differently based on whether or not the first vital metric is within the threshold range provides the user with improved visual feedback about a state of the system (e.g., the system has determined that the first vital metric is inside or outside the threshold range). Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the representation of the first vital metric in the second visual manner further comprises: in accordance with a determination that the first vital metric is above the threshold range for first day (e.g., the measured value for the first vital metric is higher than the upper limit of the threshold range), displaying the representation of the first vital metric above the first display region (e.g., region 610b above region 610a) (e.g., displaying the representation of the first vital metric in a second display region that is above the first display region); and in accordance with a determination that the first vital metric is below the threshold range for first day (e.g., the measured value for the first vital metric is lower than the lower limit of the threshold range), displaying the representation of the first vital metric below the first display region (e.g., region 610c below region 610a) (e.g., displaying the representation of the first vital metric in a third display region that is below the first display region). Displaying the representation of the first vital metric differently based on whether or not the first vital metric is within the threshold range provides the user with improved visual feedback about a state of the system (e.g., the system has determined that the first vital metric is inside or outside the threshold range). Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, concurrently displaying the representation of the first vital metric (e.g., 612a, 612b, 612c, 612d, and/or 612e) and the representation of the second vital metric (e.g., 612a, 612b, 612c, 612d, and/or 612e) comprises: in accordance with a determination that the second vital metric falls within a second threshold range for the first day (e.g., a second threshold range that is the same as or different from the threshold range), displaying the representation of the second vital metric in the first visual manner (e.g., within region 610a) to indicate that the second vital metric falls within the second threshold range for the first day; and in accordance with a determination that the second vital metric falls outside the second threshold range for the first day, displaying the representation of the second vital metric in the second visual manner (e.g., within region 610a) to indicate that the second vital metric falls outside the second threshold range for the first day. Displaying the representation of the second vital metric differently based on whether or not second first vital metric is within a threshold range provides the user with improved visual feedback about a state of the system (e.g., the system has determined that the second vital metric is inside or outside the threshold range). Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, in response to detecting the first user input: in accordance with a determination that no vital metric of the two or more vital metrics is selected when the first user input was received (e.g., in FIG. 6E, no one vital metric is selected when suer input 620 is received), the computer system displays, via the one or more display generation components, a third weekly metrics user interface (e.g., 622 in FIG. 6F), wherein: the third weekly metrics user interface is different from the first weekly metrics user interface and the second weekly metrics user interface (e.g., 622 in FIGS. 6H1 through 6H5); the third weekly metrics user interface includes a first set of user interface objects (e.g., one or more user interface objects) (e.g., 626a, 626b, 626c, 626d, 626c, 626f, and/or 626g) that are representative of the two or more vital metrics for the first day; and the third weekly metrics user interface includes a second set of user interface objects (e.g., one or more user interface objects) that are representative of the two or more vital metrics for a second day different from the first day (e.g., 626a, 626b, 626c, 626d, 626c, 626f, and/or 626g). In some embodiments, the first set of user interface objects includes a first user interface object that is representative of a plurality of the two or more vital metrics (e.g., a single object that is representative of multiple vital metrics). In some embodiments, the third weekly metrics user interface includes a third set of user interface objects (e.g., one or more user interface objects) that are representative of the two or more vital metrics for a third day different from the first day and the second day. In some embodiments, the third weekly metrics user interface includes a fourth set of user interface objects (e.g., one or more user interface objects) that are representative of the two or more vital metrics for a fourth day different from the first day, the second day, and the third day. Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the two or more vital metrics includes two or more of: heart rate, respiratory rate, body temperature, blood oxygen level, and sleep duration. Displaying different weekly metrics user interfaces for different vital metrics based on which vital metric is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a time user interface (e.g., 638) (e.g., a user interface that includes an analog and/or digital indication of time, a clock face user interface, a watch face user interface, a reduced-power screen, a wake screen, and/or a lock screen), wherein the time user interface includes a first displayed user interface object (e.g., 638d) that corresponds to the daily metrics user interface (e.g., 608) (e.g., a first watch face complication). While displaying the time user interface, the computer system detects, via the one or more input devices, a selection input (e.g., one or more user inputs, one or more touch inputs, one or more tap inputs, one or more hardware control inputs, one or more button presses, one or more rotational inputs, one or more gesture inputs, and/or one or more spoken inputs) corresponding to selection of the first displayed user interface object (e.g., selection of complication 638d). In response to detecting the selection input corresponding to selection of the first displayed user interface object: the computer system displays, via the one or more display generation components, the daily metrics user interface (e.g., 608). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first displayed user interface object (e.g., 638d) displays a representation of the daily metrics user interface (e.g., displays the daily metrics user interface and/or a smaller version of the daily metrics user interface) (e.g., 608). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system (e.g., 600 and/or 640) displays, via the one or more display generation components, an application selection user interface (e.g., 680), wherein the application selection user interface includes: a representation of a first application that, when selected, causes the computer system to open the first application (e.g., one of applications icons 682a-682m); a representation of a second application that, when selected, causes the computer system to open the second application (e.g., one of application icons 682a-682m); and a second displayed user interface object (e.g., 684a) (e.g., a widget and/or complication) that corresponds to the daily metrics user interface (e.g., 648). While displaying the application selection user interface, the computer system detects, via the one or more input devices, a selection input (e.g., one or more user inputs, one or more touch inputs, one or more tap inputs, one or more hardware control inputs, one or more button presses, one or more rotational inputs, one or more gesture inputs, and/or one or more spoken inputs) corresponding to selection of the second displayed user interface object (e.g., user input selecting widget 684a). In response to detecting the selection input corresponding to selection of the second displayed user interface object, the computer system displays, via the one or more display generation components, the daily metrics user interface (e.g., 648). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the second displayed user interface object (e.g., 684a) displays a representation of the daily metrics user interface (e.g., 648) (e.g., displays the daily metrics user interface and/or a smaller version of the daily metrics user interface). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a lock screen transition user interface (e.g., 676), wherein the lock screen transition user interface is indicative of the computer system (e.g., 640) having transitioned from a locked state to an unlocked state (e.g., an unlocked state that has access to more features than the locked state) (e.g., having transitioned from the locked state to the unlocked state in response to receiving user authentication information (e.g., passcode-based authentication and/or biometric authentication)) and the lock screen transition user interface includes a third displayed user interface object (e.g., 678c) (e.g., a widget and/or complication) that corresponds to the daily metrics user interface (e.g., 648). While displaying the locked screen transition user interface, the computer system detects, via the one or more input devices, a selection input (e.g., one or more user inputs, one or more touch inputs, one or more tap inputs, one or more hardware control inputs, one or more button presses, one or more rotational inputs, one or more gesture inputs, and/or one or more spoken inputs) corresponding to selection of the third displayed user interface object (e.g., selection of complication 678c). In response to detecting the selection input corresponding to selection of the third displayed user interface object, the computer system displays, via the one or more display generation components, the daily metrics user interface (e.g., 648). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the third displayed user interface object (e.g., 678c) displays a representation of the daily metrics user interface (e.g., 648) (e.g., displays the daily metrics user interface and/or a smaller version of the daily metrics user interface). Displaying a user interface object that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system (e.g., 600 and/or 640) receives (e.g., via one or more sensors of the computer system and/or one or more sensors of an external device) second vital metric information that includes information pertaining to the two or more vital metrics of a user of the computer system for a second day different from the first day. In response to receiving the second vital metric information: in accordance with a determination that at least two of the two or more vital metrics are outside of a threshold range for the second day, the computer system displays, via the one or more display generation components, a first notification (e.g., 618) (e.g., a pop-up notification and/or banner notification) (e.g., a notification that is overlaid on another user interface (e.g., a user interface corresponding to a first application)) indicating that at least two or more of the two or more vital metrics are outside of the threshold range for the second day. In some embodiments, in response to receiving the second vital metric information: in accordance with a determination that less than two of the two or more vital metrics (e.g., only one and/or none of the two or more vital metrics) are outside of the threshold range for the second day, the computer system forgoes displaying the first notification (e.g., does not display any notification pertaining to the second vital metric information). In some embodiments, the threshold range encompasses multiple different threshold ranges for different vital metrics. In some embodiments, the threshold range differs for different vital metrics in absolute values, but is still the same for different vital metrics in that the threshold range is indicative of whether each respective vital metric is within an interquartile range and/or a standard deviation range of a baseline and/or target value for the respective vital metric (e.g., within 1.5 IQR of a baseline and/or target value; within Q3+1.5IQR and/or Q1−1.5IQR). As such, even though the baseline and/or target values and the absolute values of the ranges will differ for different vital metrics, the different vital metrics can be graphed in the same region based on how far the vital metrics fall from their respective target values based on interquartile range, percentage deviation, and/or standard deviation. Displaying a notification when two or more vital metrics are outside of their baseline range provides the user with visual feedback about a state of the system (e.g., the system has detected that two or more vital metrics are outside of baseline). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first notification (e.g., 618), the computer system detects, via the one or more input devices, a selection input (e.g., one or more user inputs, one or more touch inputs, one or more tap inputs, one or more hardware control inputs, one or more button presses, one or more rotational inputs, one or more gesture inputs, and/or one or more spoken inputs) corresponding to selection of the first notification (e.g., user input 619). In response to detecting the selection input corresponding to selection of the first notification, the computer system displays, via the one or more display generation components, the daily metrics user interface (e.g., 608). Displaying a notification that, when selected, causes the computer system to display the daily metrics user interface allows a user to access the daily metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 700 (e.g., FIG. 7) are also applicable in an analogous manner to the methods described below. For example, methods 800, 900, and/or 1100 optionally include one or more of the characteristics of the various methods described above with reference to method 700. For example, in some embodiments, the same computer system is used to perform methods 700, 800, 900, and/or 1100. In another example, in some embodiments, the alert recited in method 900 pertains to data shown in the metrics user interfaces and/or workload user interfaces recited in methods 700, 800, and/or 1100. For brevity, these details are not repeated below.

FIG. 8 is a flow diagram illustrating a method for tracking and providing user health metrics using a computer system in accordance with some embodiments. Method 800 is performed at a computer system (e.g., 100, 300, 500, 600, and/or 640) (e.g., a smart phone, a smart watch, a tablet, a laptop, a desktop, a wearable device, wrist-worn device, and/or head-mounted device) that is in communication with one or more display generation components (e.g., 602 and/or 642) (e.g., a display, a touch-sensitive display, and/or a display controller) (and, optionally, one or more input devices (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor)). Some operations in method 800 are, optionally, combined, the orders of some operations are, optionally, changed, and some operations are, optionally, omitted.

As described below, method 800 provides an intuitive way for tracking and providing user health metrics. The method reduces the cognitive burden on a user for tracking and accessing user health metrics, thereby creating a more efficient human-machine interface. For battery-operated computing devices, enabling a user to access health data faster and more efficiently conserves power and increases the time between battery charges.

In some embodiments, the computer system (e.g., 600 and/or 640) displays (802), via the one or more display generation components (e.g., 602 and/or 642), a metrics user interface (e.g., 608, 622, 648, and/or 656) that is representative of a first duration of time (e.g., one day, one or more days, two days, three days, five days, and/or a week (e.g., a first calendar week and/or a first seven-day period)), wherein: the metrics user interface includes (804) a first region that is representative of a first day (e.g., a first calendar day and/or a first 24-hour period of time) of the first duration of time (e.g., in user interface chart 608; in user interface 622, the region in chart 624 corresponding to an individual day (e.g., above W, T, F, S, S, M, and/or T in FIG. 6F); in user interface 648, chart 650; and/or in user interface 656, the region in chart 658 corresponding to an individual day (e.g., above SUN, MON, TUE, WED, THU, FRI, and/or SAT in chart 658 in FIG. 6X)). In some embodiments, the first region includes (806) a first set of objects (e.g., one or more objects) that are representative of a set of vital metrics for the first day (e.g., 612a-612e in chart 610; 626a, 626b, 626c, 626d, 626c, 626f, 626g, 626g-1, and/or 626g-2 in chart 624 in FIG. 6F; 652a-652e in chart 650; and/or 660a, 660b, 660c, 660d, 660c, 660f, and/or 660g in chart 658) (e.g., in some embodiments, the first region is not representative of and/or correspond to any other day), wherein the first set of vital metrics includes two or more vital metrics (e.g., heart rate, respiratory rate, body temperature, blood-oxygen level, and/or sleep duration). In some embodiments, displaying the metrics user interface includes (808): in accordance with a determination that at least one or more vital metrics of the set of vital metrics are within a threshold range for the first day, displaying (810) a first subset of the first set of objects (e.g., one or more objects) within a first section (e.g., 610a, 624a, 650a, and/or 658a) of the first region, wherein the first section of the first region is representative of the threshold range (e.g., objects displayed within the first section of the first region are indicative of one or more metrics being within the threshold range on the first day); and in accordance with a determination that at least one or more vital metrics of the set of vital metrics are outside the threshold range for the first day, displaying (812) a second subset of the first set of objects (e.g., one or more objects) within a second section (e.g., 610b, 610c, 624b, 624c, 650b, 650c, 658b, and/or 658c) of the first region, wherein the second section of the first region is different from the first section of the first region and is indicative of being outside of the threshold range. In some embodiments, the second subset of the first set of objects is distinct and/or separate from the first subset of the first set of objects. In some embodiments, the second subset of the first set of objects is part of and/or joined with the first subset of the first set objects, but extends into the second section of the first region. In some embodiments, the threshold range encompasses multiple different threshold ranges for different vital metrics. In some embodiments, the threshold range differs for different vital metrics in absolute values, but is still the same for different vital metrics in that the threshold range is indicative of whether each respective vital metric is within an interquartile range and/or a standard deviation range of a baseline and/or target value for the respective vital metric (e.g., within 1.5 IQR of a baseline and/or target value; and/or within Q3+1.5IQR and Q1−1.5IQR). As such, even though the baseline and/or target values and the absolute values of the ranges will differ for different vital metrics, the different vital metrics can be graphed in the same region based on how far the vital metrics fall from their respective target values based on interquartile range, percentage deviation, and/or standard deviation. Displaying user interface objects in different regions of a user interface based on whether vital metries are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the metrics user interface further includes: in accordance with a determination that none of the set of vital metrics are outside the threshold range for the first day, forgoing displaying the second subset of the first set of objects within the second section of the first region (e.g., in FIG. 6B, there are no outliers and no objects in regions 610b and/or 610c; and/or in FIG. 6F, for Wednesday through Monday, there are no outliers and no objects in regions 624b or 624c for those days) (e.g., forgoing displaying any objects and/or any objects representative of vital metrics within the second section of the first region). Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the first subset of the first set of objects within the first section of the first region includes displaying a first object (e.g., a single object and/or a single shape; and/or an unbroken object); and the first object is representative of a plurality of vital metrics of the set of vital metrics, including a first vital metric (e.g., heart rate, respiratory rate, body temperature, blood-oxygen level, and/or sleep duration) and a second vital metric (e.g., heart rate, respiratory rate, body temperature, blood-oxygen level, and/or sleep duration) different from the first vital metric (e.g., object 626a, 626b, 626c, 626d, 626e, and/or 626f are representative of multiple vital metrics). Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, a first end (e.g., a top end, a bottom end, a right end, or a left end) of the first object is representative of the first vital metric (e.g., the position of the first end of the first object within the first section of the first region is representative of the first vital metric and/or is representative of how close the first vital metric is to a top boundary and/or a top value of the threshold range) (e.g., the top end of object 626a, 626b, 626c, 626d, 626e, and/or 626f is representative of a first vital metric); and a second end (e.g., a top end, a bottom end, a right end, or a left end) of the first object different from the first end (e.g., opposite the first end) is representative of the second vital metric (e.g., the position of the second end of the first object within the first section of the first region is representative of the second vital metric and/or is representative of how close the second vital metric is to a bottom boundary and/or a bottom value of the threshold range) (e.g., the bottom end of object 626a, 626b, 626c, 626d, 626e, and/or 626f is representative of a second vital metric). Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the set of vital metrics includes two or more of: heart rate, respiratory rate, body temperature, blood oxygen level, and sleep duration. Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the metrics user interface (e.g., 622) includes a second region that is separate from the first region and is representative of a second day (e.g., a second calendar day and/or a second 24-hour period of time) of the first duration of time different from the first day (e.g., region above “W” in FIG. 6F is representative of a first day, Wednesday, and the region above “F” in FIG. 6F is representative of a second day, Friday); the second region includes a second set of objects (e.g., 626a, 626b, 626c, 626d, 626e, 626f, 626g, 626g-1, and/or 626g-2) (e.g., one or more objects) that are separate from the first set of objects and are representative of the set of vital metrics for the second day (e.g., in some embodiments, the second region is not representative of and/or correspond to any other day); and displaying the metrics user interface includes: in accordance with a determination that at least one or more vital metrics of the set of vital metrics are within the threshold range for the second day, displaying a first subset of the second set of objects within a first section (e.g., region 624a) of the second region (the area above the letter representing a particular day in chart 624), wherein the first section of the second region is representative of the threshold range (e.g., objects displayed within the first section of the second region are indicative of one or more metrics being within the threshold range on the second day); and in accordance with a determination that at least one or more vital metrics of the set of vital metrics are outside the threshold range for the second day, displaying a second subset of the second set of objects within a second section (e.g., 624b and/or 624c) of the second region (e.g., the area above the letter representing a particular day in chart 624), wherein the second section of the second region is different from the first section of the second region and is indicative of being outside of the threshold range. In some embodiments, the second subset of the second set of objects is distinct and/or separate from the first subset of the second set of objects. In some embodiments, the second subset of the second set of objects is part of and/or joined with the first subset of the second set of objects, but extends into the second section of the second region. In some embodiments, the threshold range encompasses multiple different threshold ranges for different vital metrics. In some embodiments, the threshold range differs for different vital metrics in absolute values, but is still the same for different vital metrics in that the threshold range is indicative of whether each respective vital metric is within an interquartile range and/or a standard deviation range of a baseline and/or target value for the respective vital metric (e.g., within 1.5 IQR of a baseline and/or target value). As such, even though the baseline and/or target values and the absolute values of the ranges will differ for different vital metrics, the different vital metrics can be graphed in the same region based on how far the vital metrics fall from their respective target values based on interquartile range, percentage deviation, and/or standard deviation. Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the metrics user interface (e.g., 622) further includes: in accordance with a determination that at least one or more vital metrics of the set of vital metrics are outside of the threshold range for the first day, displaying the first set of objects in a first color (e.g., including displaying the first subset and the second subset of the first set of objects in the first color); and in accordance with a determination that none of the vital metrics of the set of vital metrics is outside of the threshold range for the first day, displaying the first set of objects in a second color different from the first color (e.g., in some embodiments, objects 626a-626f in FIG. 6F are displayed in a different color than objects 626g, 626g-1, and 626g-2 based on Tuesday (represented by objects 626g, 626g-1, and 626g-2) having one or more outliers, and Wednesday through Monday (represented by objects 626a-626f, respectively) having no outliers). Displaying user interface objects in different regions of a user interface and in different colors based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the metrics user interface (e.g., 622) in a first manner (e.g., with a first set of visual characteristics) indicating that the first day is selected (e.g., the first set of objects representative of the set of vital metrics for the first day are selected) and the second day is not selected (e.g., in FIG. 6M, Tuesday is selected), the computer system detects rotation (e.g., 630) of a rotatable input mechanism (e.g., 604) (e.g., a physical rotatable input mechanism and/or a physically rotatable input mechanism). In response to detecting rotation of the rotatable input mechanism, the computer system displays the metrics user interface in a second manner (e.g., with a second set of visual characteristics) indicating that the second day is selected (e.g., the second set of objects representative of the set of vital metrics for the second day are selected) and the first day is not selected (e.g., FIG. 6N1, object 626g is no longer darkened and object 626f is now darkened). Allowing a user to select different days by rotating a rotatable input mechanism allows a user to perform these operations without displaying additional user interface objects, which conserves display space. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while the first day is selected and while displaying the metrics user interface in the first manner, the computer system displays, within the metrics user interface, a textual indication (e.g., alpha-numerical indication) of the number of vital metrics of the set of vital metrics that are outside the threshold range for the first day without displaying a textual indication of the number of vital metrics of the set of vital metrics that are outside the threshold range for the second day (e.g., in FIG. 6P, platter 622b displays how many outliers there are for Saturday); and while the second day is selected and while displaying the metrics user interface in the second manner, displaying, within the metrics user interface, a textual indication (e.g., alpha-numerical indication) of the number of vital metrics of the set of vital metrics that are outside the threshold range for the second day without displaying the textual indication of the number of vital metrics of the set of vital metrics that are outside the threshold range for the first day (e.g., had a different day been selected in FIG. 6P, platter 622b would depict how many outliers there are on the different selected day). Allowing a user to select different days by rotating a rotatable input mechanism allows a user to perform these operations without displaying additional user interface objects, which conserves display space. Furthermore, doing so also enhances the operability of the system and makes the user-system interface more efficient (e.g., by preventing erroneous inputs and helping the user to provide proper inputs and reducing errors) which, additionally, redaces power usage and improves the battery life of the device by enabling the user to use the system more quickly and efficiently.

In some embodiments, while displaying the metrics user interface (e.g., 622) (e.g., a weekly metrics user interface and/or a multi-day metrics user interface), the computer system detects, via one or more input devices, a first user input (e.g., one or more user inputs, one or more touch inputs, one or more tap inputs, one or more hardware control inputs, one or more button presses, one or more rotational inputs, one or more gesture inputs, and/or one or more spoken inputs) corresponding to a user request to display a second metrics user interface (e.g., a daily metrics user interface that is representative of a single calendar day and/or a single 24-hour period) different from the metrics user interface (e.g., user input 634a and/or user input 627). In response to detecting the first user input: in accordance with a determination that the first day was selected (e.g., the first set of objects representative of the set of vital metrics for the first day were selected) when the first user input was received, the computer system displays, via the one or more display generation components, a first respective metrics user interface (e.g., 608), wherein the first respective metrics user interface displays first vital metric information that is representative of the first day and the set of vital metrics for the first day without displaying the set of vital metrics for the second day (e.g., in FIG. 6Q, in response to user input 634a when Saturday is selected, computer system 600 displays user interface 608 for Saturday); and in accordance with a determination that the second day was selected (e.g., the second set of objects representative of the set of vital metrics for the second day were selected) when the first user input was received, displaying, via the one or more display generation components, a second respective metrics user interface, wherein the second respective metrics user interface displays second vital metric information that is representative of the second day and the set of vital metrics for the second day without displaying the set of vital metrics for the first day (e.g., in FIGS. 6P-6Q, had a different day been selected in FIG. 6P, computer system 600 would display user interface 608 for the different selected day in FIG. 6Q). Displaying different metrics user interfaces for different days based on which day is selected when user input is received enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the metrics user interface (e.g., 622) includes a third region that is separate from the first region and the second region and is representative of a third day (e.g., a third calendar day and/or a third 24-hour period of time) of the first duration of time different from the first day and the second day; the third region includes a third set of objects (e.g., one or more objects) that are separate from the first set of objects and the second set of objects and are representative of the set of vital metrics for the third day (e.g., in some embodiments, the third region is not representative of and/or correspond to any other day); and displaying the metrics user interface includes: in accordance with a determination that at least one or more vital metrics of the set of vital metrics are within the threshold range for the third day, displaying a first subset of the third set of objects within a first section (e.g., region 624a) of the third region, wherein the first section of the third region is representative of the threshold range (e.g., objects displayed within the first section of the third region are indicative of one or more metrics being within the threshold range on the third day); and in accordance with a determination that at least one or more vital metrics of the set of vital metrics are outside the threshold range for the third day, displaying a second subset of the third set of objects within a second section (e.g., region 624b and/or region 624c) of the third region, wherein the second section of the third region is different from the first section of the third region and is indicative of being outside of the threshold range. Displaying user interface objects in different regions of a user interface based on whether vital metrics are within a threshold range or outside of the threshold range provides the user with visual feedback about a state of the computer system (e.g., the computer system has detected that one or more metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a time user interface (e.g., 638) (e.g., a user interface that includes an analog and/or digital indication of time, a clock face user interface, a watch face user interface, a reduced-power screen, a wake screen, and/or a lock screen), wherein: the time user interface includes a first displayed user interface object (e.g., 638d and/or 638e) that corresponds to the metrics user interface (e.g., 608 and/or 622) (e.g., a first watch face complication); and the first displayed user interface object displays a representation of the metrics user interface (e.g., displays the metrics user interface and/or a smaller version of the metrics user interface). In some embodiments, the first displayed user interface object, when selected, causes the computer system to cease display of the time user interface and display the metrics user interface. Displaying the metrics user interface as a watch complication allows a user to access the metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a lock screen transition user interface (e.g., 676), wherein: the lock screen transition user interface is indicative of the computer system (e.g., 640) having transitioned from a locked state to an unlocked state (e.g., an unlocked state that has access to more features than the locked state) (e.g., having transitioned from the locked state to the unlocked state in response to receiving user authentication information (e.g., passcode-based authentication and/or biometric authentication)); the lock screen transition user interface includes a second displayed user interface object (e.g., 678c and/or 678d) (e.g., a widget and/or complication) that corresponds to the metrics user interface (e.g., 648 and/or 656); and the second displayed user interface object displays a representation of the metrics user interface (e.g., displays the metrics user interface and/or a smaller version of the metrics user interface). In some embodiments, the second displayed user interface object, when selected, causes the computer system to cease display of the lock screen transition user interface and display the metrics user interface. Displaying the metrics user interface as a lock screen complication allows a user to access the metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, an application selection user interface (e.g., 680), wherein: the application selection user interface includes: a representation of a first application that, when selected, causes the computer system to open the first application (e.g., one of 682a-682m); a representation of a second application that, when selected, causes the computer system to open the second application (e.g., one of 682a-682m); and a third displayed user interface object (e.g., 684a and/or 684b) (e.g., a widget and/or complication) that corresponds to the metrics user interface (e.g., 648 and/or 656); and the third displayed user interface object displays a representation of the metrics user interface (e.g., displays the metrics user interface and/or a smaller version of the metrics user interface). In some embodiments, the third displayed user interface object, when selected, causes the computer system to cease display of application selection user interface and display the metrics user interface. Displaying the metrics user interface as a lock screen complication allows a user to access the metrics user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 800 (e.g., FIG. 8) are also applicable in an analogous manner to the methods described above and/or below. For example, methods 700, 900, and/or 1100 optionally include one or more of the characteristics of the various methods described above with reference to method 800. For example, in some embodiments, the same computer system is used to perform methods 700, 800, 900, and/or 1100. In another example, in some embodiments, the alert recited in method 900 pertains to data shown in the metrics user interfaces and/or workload user interfaces recited in methods 700, 800, and/or 1100. For brevity, these details are not repeated below.

FIG. 9 is a flow diagram illustrating a method for tracking and providing user health metrics using a computer system in accordance with some embodiments. Method 900 is performed at a computer system (e.g., 100, 300, 500, 600, and/or 640) (e.g., a smart phone, a smart watch, a tablet, a laptop, a desktop, a wearable device, wrist-worn device, and/or head-mounted device) that is in communication with one or more display generation components (e.g., 602 and/or 642) (e.g., a display, a touch-sensitive display, and/or a display controller) and one or more input devices (e.g., 602, 604, 642, and/or 644a-644c) (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor). Some operations in method 900 are, optionally, combined, the orders of some operations are, optionally, changed, and some operations are, optionally, omitted.

As described below, method 900 provides an intuitive way for tracking and providing user health metrics. The method reduces the cognitive burden on a user for tracking and accessing user health metrics, thereby creating a more efficient human-machine interface. For battery-operated computing devices, enabling a user to access health data faster and more efficiently conserves power and increases the time between battery charges.

In some embodiments, the computer system receives (902), via the one or more input devices, first vital metric information pertaining to a user, wherein the first vital metric information pertaining to the user corresponds to two or more vital metrics (e.g., heart rate, respiratory rate, blood-oxygen level, body temperature, and/or sleep duration) (in some embodiments, the first vital metric information is measured and/or collected by one or more sensors of the computer system (e.g., over a duration of two or more days (e.g., three days, five days, seven days, ten days, fourteen days, or twenty days)). The computer system determines (904), based on the first vital metric information, baseline value ranges for the two or more vital metrics, including a first baseline value range for a first vital metric (e.g., a first baseline value range that includes a minimum value and a maximum value for the first vital metric) of the two or more vital metrics and a second baseline value range for a second vital metric of the two or more vital metrics (e.g., a second baseline value range that includes a minimum value and a maximum value for the second vital metric) (e.g., FIG. 6A). Subsequent to determining the baseline value ranges for the two or more vital metrics, the computer system receives (906), via the one or more input devices, second vital metric information pertaining to the user (in some embodiments, the second vital metric information is measured and/or collected by one or more sensors of the computer system) (e.g., FIG. 6C). In response to receiving the second vital metric information pertaining to the user (908): in accordance with a determination that the second vital metric information is indicative of the first vital metric falling outside of the first baseline value range, the computer system displays (910), via the one or more display generation components, a first alert (e.g. 618) that is indicative of the first vital metric falling outside of the first threshold range, wherein the first alert identifies a first potential reason for the first vital metric falling outside of the first threshold range (e.g., 618a and/or 618d) (e.g., the user is pregnant, the user is sick, the user has traveled recently, and/or the user has traveled to a different altitude recently). In some embodiments, in response to receiving the second vital metric information pertaining to the user: in accordance with a determination that the second vital metric information is not indicative of the first vital metric falling outside of the first baseline value range, the computer system forgoes displaying the first alert. Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first alert (e.g., 618) identifies the first vital metric (e.g., the first alert indicates which vital metric and/or which vital metrics are outside of their baseline ranges) (e.g., 618a identifies which vitals are outside baseline; and chart 610 identifies which vitals are outside baseline). Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first alert (e.g., 618) identifies the second vital metric and indicates that the second vital metric is within the second baseline value range (e.g., chart 6I in notification 618 indicates which vitals are within baseline and which vitals are outside baseline). In some embodiments, the first alert identifies one or more vital metrics that are not outside of their baseline range. Displaying an alert when a user's vital metrics are determined to be outside of their baseline ranges, and also displaying within the alert an indication of which vital metrics are not outside of baseline, provides the user with visual feedback about a state of the computer system (e.g., the computer system has detected that one or more metrics are outside of their baseline range and has detected that one or more metrics are not outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a time user interface (e.g., 614) (e.g., a user interface that includes an analog and/or digital indication of time, a clock face user interface, a watch face user interface, a reduced-power screen, a wake screen, and/or a lock screen). In response to receiving the second vital metric information pertaining to the user: in accordance with a determination that the second vital metric information is indicative of the first vital metric falling outside of the first baseline value range, the computer system ceases display of the time user interface and displaying the first alert (e.g., FIGS. 6C-6D). Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first alert (e.g., 618), the computer system detects, via the one or more input devices, a dismiss input (e.g., one or more user inputs, one or more touch inputs, one or more hardware inputs, one or more button presses, one or more rotational inputs, one or more gesture inputs, and/or one or more spoken inputs) corresponding to a user request to dismiss the first alert (e.g., a user input selecting dismiss option 618c). In response to detecting the dismiss input: the computer system ceases display of the first alert (e.g., 618); and displays, via the one or more display generation components, the time user interface (e.g., 614). Providing with the user with an option to dismiss the first alert enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first alert (e.g., 618), the computer system detects, via the one or more input devices, a first user input (e.g., 619) (e.g., one or more user inputs, one or more touch inputs, one or more hardware inputs, one or more button presses, one or more rotational inputs, one or more gesture inputs, and/or one or more spoken inputs) corresponding to the first alert (e.g., a first user input corresponding to selection of the first alert and/or corresponding to selection of a first user interface object within the first alert). In response to detecting the first user input, the computer system ceases display of the first alert; and displays, via the one or more display generation components, a metrics user interface (e.g., 608) (e.g., a daily metrics user interface, a weekly metrics user interface, and/or other metrics user interface that displays information corresponding to the first alert) that includes vital metric information corresponding to the second vital metric information, including a visual indication that the first vital metric is outside of the first baseline value range (e.g., FIGS. 6D-6E). Providing the user with an option to display a metrics user interface allows the user to perform these operations with fewer inputs. Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a first application user interface that corresponds to a first application (e.g., is generated by the first application) (e.g., a first application running on the computer system) (e.g., in some embodiments, in FIG. 6C, rather than display time user interface 614, computer system 600 displays a user interface by a first application). In response to receiving the second vital metric information pertaining to the user: in accordance with a determination that the second vital metric information is indicative of the first vital metric falling outside of the first baseline value range, the computer system ceases display of the first application user interface and displaying the first alert (e.g., from FIGS. 6C-6D, computer system 600 ceases display of the displayed user interface to display notification 618). Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the first alert (e.g., 618), the computer system detects, via the one or more input devices, a first set of user inputs (e.g., one or more user inputs, one or more touch inputs, one or more hardware inputs, one or more button presses, one or more rotational inputs, one or more gesture inputs, and/or one or more spoken inputs) corresponding to the first alert (e.g., a first user input corresponding to selection of the first alert and/or corresponding to selection of a first user interface object within the first alert). In response to detecting the first user input: in accordance with a determination that the first set of user inputs is a first type of user input (e.g., a user input that selects a first object within the first alert and/or a user input directed to a first region of the first alert) (e.g., selecting option 618b), the computer system displays, via the one or more display generation components, a metrics user interface (e.g., 608) (e.g., a daily metrics user interface, a weekly metrics user interface, and/or other metrics user interface that displays information corresponding to the first alert) that includes vital metric information corresponding to the second vital metric information, including a visual indication that the first vital metric is outside of the first baseline value range without displaying the first application user interface; and in accordance with a determination that the first set of user inputs is a second type of user input different from the first type of user input (e.g., a user input that selects a second object within the first alert and/or a user input directed to a second region of the first alert) (e.g., user input selecting option 618c), the computer system displays the first application user interface without displaying the metrics user interface (e.g., in FIG. 6D, if option 618c is selected, the computer system returns to displaying the user interface it was previously displaying in FIG. 6C). Providing the user options to either display a metrics user interface or dismiss the first alert and re-display the first application user interface allows the user to perform these operations with fewer inputs. Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first vital metric is a heart rate metric. Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first vital metric is a respiratory rate metric. Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first vital metric is a body temperature metric. Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the first vital metric is a blood oxygen level metric. Displaying an alert when a user's vital metrics are determined to be outside of their baseline ranges provides the user with visual feedback about a state of the computer system (e.g., the computer system has detected that one or more metrics are outside of their baseline range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the second vital metric information pertaining to the user includes information pertaining to the first vital metric, information pertaining to the second vital metric, and information pertaining to a sleep duration vital metric; and the sleep duration vital metric corresponds to a third baseline value range (e.g., a third baseline value range that includes a minimum value and a maximum value for the sleep duration metric). In some embodiments, in response to receiving the second vital metric information pertaining to the user: in accordance with a determination that the second vital metric information is indicative of the sleep duration vital metric falling outside of the third baseline value range, the computer system forgoes displaying the first alert (e.g., forgoing displaying any alert that is indicative of the sleep duration metric falling outside of the third baseline value range) (e.g., in some embodiments, if the sleep duration metrics is outside of baseline, computer system 600 does not display notification 618; but if one or more of the other vital metrics are outside of baseline, computer system 600 does display notification 618). In some embodiments, when the sleep duration vital metric is determined to be outside of the baseline value range, the computer system does not display and/or output a corresponding alert. However, when other vital metrics are determined to be outside of their respective baseline value ranges, the computer system does display and/or output a corresponding alert. In some embodiments, in response to receiving the second vital metric information pertaining to the user: in accordance with a determination that the second vital metric information is indicative of the sleep duration vital metric falling within the third baseline value range, the computer system forgoes displaying the first alert (e.g., forgoes displaying any alert that is indicative of the sleep duration metric falling outside of the third baseline value range). Forgoing displaying an alert when the user's sleep duration metric is outside of the baseline value range conserves computing resources, such as display space, processing bandwidth, and battery power, and also avoids displaying content to the user that is not of interest and/or is not useful to the user (e.g., as the user already knows how long the user has slept).

Note that details of the processes described above with respect to method 900 (e.g., FIG. 9) are also applicable in an analogous manner to the methods described above and/or below. For example, methods 700, 800, and/or 1100 optionally include one or more of the characteristics of the various methods described above with reference to method 900. For example, in some embodiments, the same computer system is used to perform methods 700, 800, 900, and/or 1100. In another example, in some embodiments, the alert recited in method 900 pertains to data shown in the metrics user interfaces and/or workload user interfaces recited in methods 700, 800, and/or 1100. For brevity, these details are not repeated below.

FIGS. 10A-10AG illustrate exemplary user interfaces for tracking and providing user health metrics, in accordance with some embodiments. The user interfaces in these figures are used to illustrate the processes described below, including the processes in FIG. 11.

FIG. 10A illustrates computer system 600, which is a smart watch with touch-sensitive display 602 and rotatable and depressible input mechanism 604. At FIG. 10A, computer system 600 displays workout selection user interface 1000. Workout selection user interface 1000 includes platter 1000a and platter 1000b. Platter 1000a, when selected, causes computer system 600 to initiate a workout session corresponding to a first workout type (e.g., outdoor run), and platter 1000b, when selected, causes computer system 600 to initiate a workout session corresponding to a second workout type (e.g., outdoor walk). At FIG. 10A, computer system 600 detects user input 1001, which is a touch input (e.g., a tap input) corresponding to selection of platter 1000a.

At FIG. 10B, in response to user input 1001, computer system 600 initiates an outdoor run workout session, and displays workout session user interface 1002. Workout session user interface 1002 is indicative of an active workout session, and displays workout metrics 1002a corresponding to the current workout session. For example, in FIG. 10B, the workout metrics include (from top down), workout session duration, workout zone (e.g., indicative of the intensity with which the user is exercising), heart rate, workout zone time (e.g., the user's time in the current workout zone), and maximum heart rate for the current workout session. Workout session user interface 1002 also include modality indication 1002b which indicates the workout type and/or workout modality of the current workout session (e.g., outdoor run).

At FIG. 10C, the workout session has progressed, and the user has been working out for 58 minutes and 12 seconds. At FIG. 10C, computer system 600 detects user input 1003, which is a swipe right touch input. At FIG. 10D, in response to user input 1003, computer system 600 displays user interface 1004. User interface 1004 includes options 1004a-1004d. Option 1004a, when selected, causes computer system 600 to end the current workout session. Option 1004b, when selected, causes computer system 600 to pause the current workout session. Option 1004c, when selected, causes computer system 600 to initiate a process for initiating a new workout session (e.g., a new workout session of a different workout type or the same workout type). Option 1004d, when selected, causes computer system 600 to mark and/or create a new segment for the current workout session. At FIG. 10D, computer system 600 detects user input 1005, which is a touch input (e.g., a tap input) corresponding to selection of option 1004a.

At FIG. 10E, in response to user input 1005, computer system 600 displays workout summary user interface 1006. Workout summary user interface 1006 displays workout metrics for the workout session, including heart rate range information 1006a, elevation gain information 1006c and workout time/duration information 1006d. Workout summary user interface 1006 also includes option 1000e that, when selected, cause computer system 600 to cease display of workout summary user interface 1006. Workout summary user interface 1006 also includes effort indication 1006b. In FIG. 10E, effort indication includes an automatically calculated effort score for the workout session. In some embodiments, the automatically calculated effort score is calculated based on various factors including one or more of: duration of the workout, the user's heart rate during the workout, distance traveled during the workout, and/or elevation gain during the workout. At FIG. 10E, computer system 600 detects user input 1007, which is a touch input (e.g., a tap input) corresponding to selection of effort indication 1006b.

At FIG. 10F, in response to user input 1007, computer system 600 displays user interface 1008. User interface 1008 allows the user to manually enter an effort score for the workout session. User interface 1008 includes platter 1008a, option 1008b and option 1008c. Platter 1008a displays the current setting for the effort score. Option 1008b, when selected, causes computer system 600 to return to user interface 1006 without changing the effort score. Option 1008c, when selected, causes computer system 600 to save the effort score for the workout session. At FIG. 10F, computer system 600 detects user input 1009, which is a rotation of rotatable and depressible input mechanism 604.

At FIG. 10G, in response to user input 1009, computer system 600 increases the effort score from 7 to 9. In some embodiments, rotating rotatable and depressible input mechanism 604 in a first rotation direction (e.g., clockwise) causes the effort score to be increased, and rotating rotatable and depressible input mechanism 604 in a second rotation direction different from the first direction (e.g., counterclockwise) causes the effort score to be decreased. At FIG. 10G, computer system 600 detects user input 1008c, which is a touch input (e.g., a tap input) corresponding to selection of option 1008c.

At FIG. 10H, in response to user input 1008c, computer system 600 re-displays workout summary user interface 1006, but effort indication 1006b has been updated to indicate the manually-entered effort score of 9.

At FIG. 10I, computer system 600 displays workout selection user interface 1000. In In FIG. 10I, workout selection user interface 1000 displays different workout platters (for example, based on the user navigating to a different portion of workout selection user interface 1000). In FIG. 10I, workout selection user interface 1000 includes platter 1000c and platter 1000d. Platter 1000c, when selected, causes computer system 600 to initiate a workout session corresponding to a strength training workout type and platter 1000d, when selected, causes computer system 600 to initiate a workout session corresponding to a different workout type (e.g., stretching). At FIG. 10I, computer system 600 detects user input 1011, which is a touch input (e.g., a tap input) corresponding to selection of platter 1000c.

At FIG. 10J, in response to user input 1011, computer system 600 initiates a strength training workout session, and displays workout session user interface 1002. Workout session user interface 1002 is indicative of an active workout session, and displays workout metrics 1002a corresponding to the current workout session and modality indication 1002b which indicates the workout type and/or workout modality of the current workout session (e.g., strength training). At FIG. 10J, computer system 600 detects user input 1012, which is a swipe right touch input.

At FIG. 10K, in response to user input 1012, computer system 600 displays user interface 1004, which includes options 1004a-1004d which were described above with reference to FIG. 10D. At FIG. 10K, computer system 600 detects user input 1013, which is a touch input (e.g., a tap input) corresponding to selection of option 1004a.

At FIG. 10L, in response to user input 1013, computer system 600 displays workout summary user interface 1006. In some embodiments, different workout types result in workout summary user interface 1006 displaying different workout metrics. For example, whereas the outdoor run workout type included elevation gain information 1006c, the strength training workout type does not include this information, and instead displayed calories burned information 1006f. Additionally, in some embodiments, certain workout types will result in automatic calculation of an effort score, whereas other types of workouts do not result in automatic calculation of an effort score. This may be the case, for example, because certain workout types yield sufficient workout metrics and/or data for computer system 600 to generate a reasonably reliable effort score (e.g., cardiovascular workouts, running workouts, biking workouts, and/or rowing workouts), whereas other workout types are more difficult to calculate a reliable or useful effort score (e.g., strength training, tennis, baseball, and/or stretching). In FIG. 10L, workout summary user interface 1006 does not include an automatically calculated effort score, but does still include effort indication 1006b (without any effort score filled in). At FIG. 10L, computer system 600 detects user input 1014, which is a touch input (e.g., a tap input) corresponding to selection of effort indication 1006b.

At FIG. 10M, in response to user input 1014, computer system 600 displays user interface 1008, which was described above. At FIG. 10M, computer system 600 detects user input 1015a, which is a rotation of rotatable and depressible input mechanism 604. At FIG. 10N, in response to user input 1015a, computer system 600 scrolls through effort level scores 1, 2, 3, and 4 to effort level score 5. At FIG. 10N, computer system 600 detects user input 1015b, which is a touch input (e.g., a tap input) corresponding to selection of option 1008c.

At FIG. 10O, in response to user input 1015b, computer system 600 displays workout summary user interface 1006 with effort indication 1006b displaying the manually-entered effort score of 5.

In some embodiments, computer system 600 uses workout information, including workout effort scores, to calculate a workload metric. In some embodiments, the workload metric is calculated for each day (e.g., each calendar day or for a 24-hour period), and is indicative of the user's energy expenditure and/or physical effort during that day. In some embodiments, the workload metric is also cumulative, such that the workload score for a particular day takes into account the workload scores for one or more days before and/or leading into the previous day. For example, in some embodiments, a first workload score for a first day is calculated based on the user's physical activity during the first day (e.g., based on duration of workouts during the first day, effort scores for workouts on the first day, based on the total number of steps taken by the user during the first day, based on the total elevation gain of the user during the first day, and/or based on total distance traveled by the user during the first day). When the first day ends, and a second day begins, a portion of the user's first workload score is deducted (e.g., a predetermined number of points is deducted, or a predetermined percentage of the first workload score is deducted), and the user begins the second day with the new work score value as an initial work score value, and the workload score for the second day increases from the initial score based on the user's physical activity during the second day (e.g., based on the same factors that were considered for calculating the first work day's workload score). And then as the second day ends and a third day begins, the second day's final workload score is reduced to calculate the third day's initial workload score, and so forth. In this way, the workload score for the current day is indicative of the cumulative toll of the user's physical activity from the current day and the days leading up to the current day. In some embodiments, a low workload score is indicative of the user's body having rested sufficiently that it is able to recover, but is also indicative of low physical activity by the user (e.g., suggestive of low gains in physical performance). In contrast, in some embodiments, a high workload score is indicative of high physical activity and high performance gains by the user, but potentially also indicative of the need for the user to recover and rest and/or a higher risk of injury.

At FIG. 10P, computer system 600 displays workload user interface 1016, which displays workload scores for the user across seven days. Workload user interface 1016 includes option 1016a, platter 1016b, platter 1016c, option 1016d, and chart 1018. Chart 1018 includes objects (e.g., data points) 1022a-1022g. Object 1022a is representative of a first day (e.g., Wednesday), and is displayed at a position (e.g. a vertical position in which a higher position is indicative of a higher workload score) in chart 1018 indicative of the user's workload score for the first day. Object 1022b is representative of a second day (e.g., Thursday), and is displayed at a position in chart 1018 indicative of the user's workload score for the second day. Objects 1022c-1022g are representative of third, fourth, fifth, sixth, and seventh days, respectively, and are displayed at positions in chart 1018 indicative of the user's workload score for each of these days. Chart 1018 also includes average score indication 1020. In some embodiments, average score indication 1020 charts the moving average (or, in some embodiments, a moving weighted average) of the user's workload score over time. Using average score indication 1020, the user is able to see the trend of how the user's average workload is moving (e.g., moving up, moving down, or remaining steady), and is also able to see whether the user's workload score for a given day is above or below the user's moving average.

Option 1016a, when selected, causes computer system 600 to cease display of workload user interface 1016. Platter 1016b displays workload score information for a currently selected day, which in FIG. 10P is the current day, Tuesday, as indicated by object 1022g being displayed in a darker color than objects 1022a-1022f. Option 1016c is a health metric (e.g., vital metric) platter that indicates whether the user's health metrics for the selected day are within the baseline range or outside of the baseline range, as discussed above with reference to FIGS. 6A-6AC. In some embodiments, platter 1016c, when selected, causes computer system 600 to display user interface 608 (e.g., a daily metrics user interface), as discussed above with reference to FIGS. 6A-6AC. Option 1016d, when selected, causes computer system 600 to display a data filtering user interface, as will be described in greater detail below. At FIG. 10P, computer system 600 detects user input 1023a, which is a rotation of rotatable and depressible input mechanism 604.

At FIG. 10Q, in response to user input 1023a, computer system 640 displays object 1022f in a darker color and larger size to indicate that Monday is now selected, and platter 1016b is updated with workload information for Monday (e.g., indicating that the user's workload score Monday was 9% above the moving average), and platter 1016c is updated with health metric information for Monday (e.g., indicating that the user's health metrics were all within baseline range on Monday). In some embodiments, rotation of rotatable and depressible input mechanism 604 in a first rotation direction (e.g., clockwise) causes the selection to move in a first direction (e.g., left), and rotation of rotatable and depressible input mechanism 604 in a second rotation direction different from the first rotation direction (e.g., counterclockwise) causes the selection to move in a second direction (e.g., right). At FIG. 10Q, computer system 600 detects user input 1023b, which is a rotation of rotatable and depressible input mechanism 604.

At FIG. 10R, in response to user input 1023b, computer system 640 displays object 1022e in a darker color and larger size to indicate that Sunday is now selected, and platter 1016b is updated with workload information for Sunday (e.g., indicating that the user's workload score Sunday was 7% above the moving average), and platter 1016c is updated with health metric information for Sunday (e.g., indicating that two of the user's health metrics were outside of baseline range on Sunday). At FIG. 10R, computer system 600 detects user input 1024, which is a touch input (e.g., a tap input) corresponding to selection of option 1016d.

At FIG. 10S, in response to user input 1024, computer system 600 displays user interface 1026. User interface 1026 includes option 1026a that, when selected, causes computer system to return to displaying user interface 1016. User interface 1026 also includes platters 1026a-1026c. In some embodiments, platters 1026a-1026c can be used to filter the data that is shown in workload user interface 1016. For example, platter 1026a, when selected, causes computer system 1016 to display workload user interface 1016 using data corresponding to all workouts during each day, but excluding non-workout activity (e.g., steps taken or distance walked); platter 1026b, when selected, causes computer system 1016 to display workload user interface 1016 using only running workout data (e.g., showing the user's running workload score based on running workouts and not using any other types of workouts); and platter 1026c, when selected, causes computer system 1016 to display workload user interface 1016 using all physical activity metrics during the day (e.g., including all workouts and all non-workout physical activities, such as steps taken and distance walked). These features will be demonstrated and discussed in greater detail below with reference to FIGS. 10AA-10AC. Platter 1026a includes option 1026a-1 that, when selected, causes computer system 600 to display representations of all workouts that were performed by the user on the selected day (e.g., the selected day was Sunday in FIG. 10R when user interface 1026 was opened). Platter 1026b includes option 1026b-1 that, when selected, causes computer system 600 to display representations of all running workouts that were performed by the user on the selected day (e.g., Sunday in FIGS. 10R-10S). Platter 1026b includes option 1026c-1 that, when selected, causes computer system 600 to display representations of all physical activity metrics on the selected day, including workouts and non-workout physical activity. At FIG. 10S, computer system 600 detects user input 1029, which is a touch input (e.g., a tap input) corresponding to selection of option 1026a-1.

At FIG. 10T, in response to user input 1029, computer system 600 displays user interface 1030. User interface 1030 includes platter 1032a, which is representative of a strength training workout completed by the user on Sunday, and platter 1032b, which is representative of an outdoor run workout completed by the user on Sunday. In some embodiments, had the user selected option 1026b-1 in FIG. 10S, the user interface would include platter 1032b (a running workout), but not platter 1032a (a non-running workout). Platter 1032a indicates that the effort score for the strength training workout was 5, and platter 1032b indicates that the effort score for the running workout was 7. At FIG. 10T, computer system 600 detects user input 1033, which is a touch input corresponding to selection of platter 1032b.

At FIG. 10U, in response to user input 1033, computer system 600 displays workout summary user interface 1034. Workout summary user interface 1034 displays workout metrics 1036a for the running workout the user completed on Sunday, and also includes effort platter 1036b that displays the effort score for the running workout. Workout summary user interface 1034 also includes option 1034a which, when selected, causes computer system 600 to close workout summary user interface 1034 and re-display user interface 1030 in FIG. 10T. At FIG. 10U, computer system 600 detects user input 1037, which is at ouch input (e.g., a tap input) corresponding to selection of platter 1036b.

At FIG. 10V, in response to user input 1037, computer system 600 displays user interface 1008, which was introduced and discussed above. In FIG. 10V, platter 1008a indicates that the current effort score for the running workout is 7. At FIG. 10V, computer system 600 detects user input 1038, which is a rotation of rotatable and depressible input mechanism 604. At FIG. 10W, in response to user input 1038, computer system 600 changes the effort score from 7 to 2. At FIG. 10W, computer system detects user input 1039, which is a touch input (e.g., a tap input) corresponding to selection of option 1008c. At FIG. 10X, in response to user input 1039, computer system 600 re-displays user interface 1034, which now shows the effort score changed to 2 in platter 1036b. At FIG. 10X, computer system 600 detects user input 1040, which is a touch input (e.g., a tap input) corresponding to selection of option 1034a.

At FIG. 10Y, in response to user input 1040, computer system 600 re-displays workload user interface 1016. However, in FIG. 10Y, based on the user changing the effort score of the running workout on Sunday from a 7 to a 2, the workload score for the user for Sunday dropped. Furthermore, due to the workload score for Sunday dropping, the workload scores for Monday and Tuesday also dropped (e.g., based on workload scores being dependent on workload scores from previous days, as discussed above). Accordingly, objects 1022e, 1022f, and 1022g have moved lower compared to FIG. 10R, and average score indication 1020 is also trending lower compared to FIG. 10R.

FIGS. 10Z-10AF depict scenarios in which user workload information is displayed on a different computer system, e.g., computer system 640. FIG. 10Z depicts computer system 640, which is a smart phone with touch-sensitive display 642 and buttons 644a-644c. At FIG. 10Z, computer system 600 displays health summary user interface 646, which was described above (e.g., with reference to FIG. 6U). In FIG. 10Z, health summary user interface 646 includes additional platter 1042, which displays user workload information for the current day. In FIG. 10Z, platter 1042 indicates that the user's workload for the current day is 12% above the average (e.g., the moving average). At FIG. 10Z, computer system 600 detects user input 1043, which is a touch input corresponding to selection of platter 1042.

At FIG. 10AA, in response to user input 1043, computer system 600 displays workload user interface 1044. Workload user interface displays chart 1048, which displays objects 1052a-1052g which, similar to objects 1022a-1022g in chart 1018, each represent a respective day, and are indicative of the user's workload score for the corresponding day. Chart 1048 also includes average score indication 1050 which, similar to average score indication 1020, indicates and/or charts the moving average of the user's workload score over time. User interface 1044 includes platter 1056, which displays the health metric information for the user for the current day, and includes representation 1056a that is representative of and/or displays chart 650 from FIG. 6V. In some embodiments, platter 1056, when selected, causes computer system 600 to display user interface 648 of FIG. 6V. User interface 1044 includes option 1044a which, when selected, causes computer system 640 to re-display user interface 646. User interface 1044 also includes a recent activities section 1054, which includes platter 1054a, representative of a traditional strength training workout completed by the user on the current day, and platter 1054b, representative of an outdoor running workout completed by the user on the current day. User interface 1044 also includes filtering options 1046a-1046c for filtering the data shown in chart 1048. In FIG. 10AA, filtering option 1046a is selected, which means that the data shown in chart 1048 includes data for all workouts performed during the week (and, optionally, excludes non-workout data, such as steps taken or distance walked). In FIG. 10AA, computer system 640 detects user input 1057, which is a touch input (e.g., a tap input) corresponding to selection of filtering option 1046b.

At FIG. 10AB, in response to user input 1057, computer system 640 updates chart 1048 to display workload information only pertaining to the user's running workouts. In some embodiments, this includes updating average score indication 1050 to display the user's average workload score based on running workouts, and updating the positions of objects 1052a-1052g to reflect the workload score for each day based only on running workouts (and, for example, excluding other types of workouts). Additionally, recent activities section 1054 is updated to include only running workouts, such that platter 1054a is no longer displayed while platter 1054b continues to be displayed. At FIG. 10AB, computer system 640 detects user input 1058, which is a touch input (e.g., a tap input) corresponding to selection of filtering option 1046c.

At FIG. 10AC, in response to user input 1058, computer system 640 updates chart 1048 to display workload information pertaining to all activities during the user's day, including all workouts and all non-workout activity (e.g., steps taken, distance walked, and/or elevation gained outside of workouts).

At FIG. 10AD, computer system 640 displays recent workouts user interface 1060. Recent workouts user interface 1060 includes representations 1062a-1062c indicative of three different workouts recently completed by the user. Recent workouts user interface 1060 also includes option 1060b that, when selected, causes computer system 640 to display representations of additional workouts (e.g., older workouts) not shown in recent workouts user interface 1060. Representation 1062a corresponds to a strength training workout that was completed today, and has an effort score of five; representation 1062b corresponds to an outdoor run workout that was completed Friday and has an effort score of seven; and representation 1062c corresponds to a Pilates workout that was completed Friday and does not yet have an effort score assigned. At FIG. 10AD, computer system 640 detects user input 1063, which is a touch input (e.g., a tap input) corresponding to selection of representation 1062a.

At FIG. 10AE, in response to user input 1063, computer system 640 displays user interface 1064, which allows the user to change the effort score for the selected workout. User interface 1064 includes option 1064a which, when selected, causes computer system 640 to re-display user interface 1060 without changing the effort score for the workout; option 1064b which, when selected, causes computer system 640 to re-display user interface 1060 and update the effort score for the workout; and platter 1064c, which displays the currently selected effort level for the workout. In some embodiments, the user can change the effort score for the workout by interacting with user interface 1064 (e.g., such as via touch input 1065), and selecting option 1064b.

In various embodiments, computer system 640 displays workload information as a widget and/or a complication in a different user interface. For example, in FIG. 10AF, computer system 640 is depicted displaying home screen user interface 680 (on the left) and displaying lock screen transition user interface 676 (in the center), both of which were previously disclosed above. On the left side of FIG. 10AF, home screen user interface includes widget 1066, which displays an indication of the user's workload score for the current day. In some embodiments, widget 1066 displays chart 1048. In the center of FIG. 10AF, lock screen transition user interface 676 includes complication 1668, which displays an indication of the user's workload score for the current day. In some embodiments, complication 1668 displays chart 1048. On the right side of FIG. 10AF, computer system 600 displays time user interface 638, which was previously discussed above. In FIG. 10AF, time user interface 638 includes complication 1070, which displays an indication of the user's workload score for the current day. In some embodiments, complication 1070 displays chart 1048.

In FIG. 10AG, computer system 600 displays time user interface 1072. Time user interface 1072 is watch face that includes current time indication 1072a and complications 1074a-1074f. In some embodiments, complications 1074a-1074f correspond to various respective applications, and display information provided by the various respective application and, in some embodiments, when selected, cause computer system 600 to display the corresponding respective application. Time user interface 1072 also includes border complication 1076 and border complication 1078. Border complication 1076 displays health metric information for the user, and indicates whether any of the user's health metrics on the current day are outside of the user's baseline range. Border complication 1076 includes region 1076a, which is representative of health metrics being within the baseline range, region 1076b, which is representative of health metrics being above the baseline range, and region 1076c, which is representative of health metrics being below the baseline range. In FIG. 10AG, object 1077a is displayed in region 1076b, indicating that one health metric is above baseline range for the current day (e.g., as measured overnight the previous night); object 1077b and object 1077c are displayed in region 1076a, indicating that two health metrics are within baseline range for the current day; and object 1077d and object 1077e are displayed in region 1076c, indicating that two health metrics are below baseline range for the current day. Border complication 1078 displays workload information for the user, and includes regions 1078a, 1078b, 1708c. Region 1708a is indicative of a workload score for the current day that is within a threshold percentage of the average score (e.g., a moving and/or weighted average score), region 1708b is indicative of a workload score for the current day that is more than the threshold percentage above the average score, and region 1708c is indicative of a workload score for the current day that is less than the threshold percentage below the average score. In FIG. 10AG, object 1079 is displayed in region 1078b, indicating that the user's current workload score for the current day is more than the threshold percentage above the average score.

FIG. 11 is a flow diagram illustrating a method for tracking and providing user health metrics using a computer system in accordance with some embodiments. Method 1100 is performed at a computer system (e.g., 100, 300, 500, 600, and/or 640) (e.g., a smart phone, a smart watch, a tablet, a laptop, a desktop, a wearable device, wrist-worn device, and/or head-mounted device) that is in communication with one or more display generation components (e.g., 602 and/or 642) (e.g., a display, a touch-sensitive display, and/or a display controller) and one or more input devices (e.g., 602, 604, 642, and/or 644a-644c) (e.g., a touch-sensitive surface, a touch-sensitive display, a button, a rotatable input mechanism, a depressible and rotatable input mechanism, a camera, an accelerometer, an inertial measurement unit (IMU), a heartrate sensor, a body temperature sensor, and/or a blood-oxygen level sensor). Some operations in method 1100 are, optionally, combined, the orders of some operations are, optionally, changed, and some operations are, optionally, omitted.

As described below, method 1100 provides an intuitive way for tracking and providing user health metrics. The method reduces the cognitive burden on a user for tracking and accessing user health metrics, thereby creating a more efficient human-machine interface. For battery-operated computing devices, enabling a user to access health data faster and more efficiently conserves power and increases the time between battery charges.

In some embodiments, the computer system detects (1102), via the one or more input devices, that a user has completed a first workout (e.g., one or more user inputs indicating that the user has completed a workout; one or more user inputs requesting to end a workout; and/or one or more movements by a user indicating that the user has completed a workout) (e.g., user input 1005 and/or user input 1013). Subsequent to detecting that the user has completed the first workout (e.g., in some embodiments, in response to detecting that the user has completed the first workout and/or based on detecting that the user has completed the first workout), the computer system detects (1104), via the one or more input devices, one or more user inputs (e.g., one or more touch inputs, one or more hardware inputs, one or more button presses, one or more rotational inputs, one or more gesture inputs, and/or one or more spoken inputs) corresponding to user entry of a first workout effort value (e.g., a numerical value and/or a magnitude value) for a workout effort metric corresponding to the first workout (e.g., a first workout effort value indicative of the user's assessment of how difficult and/or strenuous the first workout was) (e.g., user inputs 1009, 1010, 1014, 1015a, and/or 1015b). After detecting the one or more user inputs corresponding to user entry of the first workout effort value for the workout effort metric corresponding to the first workout (e.g., in some embodiments, in response to detecting the one or more user inputs corresponding to user entry of the first value and/or based on detecting the one or more user inputs corresponding to user entry of the first value), the computer system displays (1106), via the one or more display generation components, a workload user interface (e.g., 1016), wherein: the workload user interface displays a first workload indication (e.g., 1022a, 1022b, 1022c, 1022d, 1022e, 1022f, and/or 1022g) that is representative of a first day (e.g., a first calendar day, a first day of the week, and/or a first 24-hour period) and is indicative of a first workload value (e.g., a numerical value and/or a magnitude value) that is calculated for the user for the first day (e.g., the display position of the first workload indication is indicative of the first workload value that is calculated for the user for the first day), wherein the first workload indication and the first workload value are determined (e.g., the first workload value is determined and/or a display position of the first workload indication is determined) based on the first workout effort value for the workout effort metric corresponding to the first workout that was entered by the user. In some embodiments, the workload value of the user on a particular day is indicative of how much effort and/or energy the user expended during the first day (e.g., in workouts and/or cumulatively over the course of the day including in workouts and outside of workouts). In some embodiments, the workload value for the user on a particular day takes into account workload values for the user in one or more previous days such that the workload value on a particular day is indicative of the user's expended effort and/or energy over the course of multiple days including the particular day. In some embodiments, the workload value of the user on a particular day is determined, at least in part, based on the duration of workouts performed on the day and the workout effort values (e.g., user-entered workout effort values and/or automatically calculated workout effort values) of workouts performed on the day. In some embodiments, the effect of a particular workout on the workload value of a user is determined based on the duration of the workout and the workout effort value (e.g., a user-entered workout effort value and/or an automatically determined workout effort value) of the workout. Allowing a user to enter workout effort information, and displaying workload information based on the user-entered workout effort information, enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, detecting that the user has completed a first workout comprises detecting, via the one or more input devices, a first user input (e.g., 1005 and/or 1013) (e.g., one or more user inputs) (e.g., one or more touch inputs, one or more hardware inputs, one or more button presses, one or more rotational inputs, one or more gesture inputs, and/or one or more spoken inputs) corresponding to a user request to end the first workout. Allowing a user to enter workout effort information, and displaying workload information based on the user-entered workout effort information, enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the one or more user inputs corresponding to user entry of a first workout effort value for the workout effort metric corresponding to the first workout comprises one or more rotations (e.g., 1009 and/or 1015a) of a rotatable input mechanism (e.g., 604) (e.g., a physical rotatable input mechanism and/or a physically rotatable input mechanism). Allowing a user to enter a workout effort metric with a rotational input allows the user to perform these operations without displaying additional user interface objects, which conserves limited display space. Furthermore, allowing a user to enter workout effort information, and displaying workload information based on the user-entered workout effort information, enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the workload user interface (e.g., 1016) further displays a second workload indication (e.g., 1022a, 1022b, 1022c, 1022d, 1022e, 1022f, and/or 1022g) that is representative of a second day (e.g., a second calendar day, a second day of the week, and/or a second 24-hour period) that is different from the first day, and further wherein the second workload indication is indicative of a second workload value that is calculated for the user for the second day (e.g., the display position of the second workload indication is indicative of the second workload value that is calculated for the user for the second day), wherein the second workload indication and the second workload value are determined (e.g., the second workload value is determined and/or a display position of the second workload indication is determined) based on a second workout effort value for the workout effort metric that was entered by the user, wherein the second workout effort value for the workout effort metric corresponds to a second workout different from the first workout (e.g., was entered by the user for a second workout different from the first workout) (e.g., a second workout that was performed and/or completed on the second day). In some embodiments, the workload user interface further displays a third workload indication (e.g., 1022a, 1022b, 1022c, 1022d, 1022e, 1022f, and/or 1022g) that is representative of a third day (e.g., a third calendar day, a third day of the week, and/or a third 24-hour period) that is different from the first day and the second day, and further wherein the third workload indication is indicative of a third workload value that is calculated for the user for the third day (e.g., the display position of the third workload indication is indicative of the second workload value that is calculated for the user for the third day), wherein the third workload indication and the third workload value are determined (e.g., the third workload value is determined and/or a display position of the third workload indication is determined) based on a third workout effort value for the workout effort metric that was entered by the user, wherein the third workout effort value for the workout effort metric corresponds to a third workout different from the first workout and the second workout (e.g., was entered by the user for a third workout different from the first workout and the second workout) (e.g., a third workout that was performed and/or completed on the third day). Allowing a user to enter workout effort information, and displaying workload information based on the user-entered workout effort information, enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the workload user interface (e.g., 1016) further includes an aggregated workload indication (e.g., 1020) that is indicative of a first set of aggregated workload values (e.g., one or more aggregated workload values), wherein the first set of aggregated workload values is determined by averaging (e.g., an average and/or a weighted average) workload values for a plurality of days (e.g., an average and/or a weighted average of workload values for a plurality of days (e.g., the previous 7 days, the previous 10 days, the previous 14 days, the previous 21 days, the previous 28 days, or the previous 30 days)). In some embodiments, the aggregated workload indication is displayed separately from the first workload indication and the second workload indication. In some embodiments, the first workload indication is a first user interface object, the second workload indication is a second user interface object different from and/or distinct from the first user interface object, and the aggregated workload indication is a third user interface object that is different from and/or distinct from the first user interface object and the second user interface object. In some embodiments, the aggregated workload value changes over time, such that the aggregated workload indication is indicative of and/or representative of a plurality of aggregated workload values. In some embodiments, the aggregated workload indication is a line that charts and/or graphs the aggregated workload value as it changes over time. Displaying an aggregated workload indication allows the user to quickly see whether their workload for a given day is above or below their typical workload, thereby improving visual feedback to the user. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the workload user interface (e.g., 1016) comprises: in accordance with a determination that the first workload value exceeds the aggregated workload value (e.g., exceeds the aggregated workload value for the first day), displaying the first workload indication in a first manner (e.g., with a first set of visual characteristics) (e.g., at a first size, with a first color, with a first shape, within a first display region, and/or at a first display position) (e.g., displaying the first workload indication above the aggregated workload indication) (e.g., in FIG. 10P, objects 1022b, 1022d, 1022e, and 1022f are displayed above line 1020); and in accordance with a determination that the first workload value does not exceed the aggregated workload value (e.g., does not exceed and/or falls below the aggregated workload value for the first day), displaying the first workload indication in a second manner different from the first manner (e.g., with a second set of visual characteristics) (e.g., at a second size, with a second color, with a second shape, within a second display region, and/or at a second display position) (e.g., displaying the first workload indication below the aggregated workload indication) (e.g., in FIG. 10P, object 1022c is displayed below line 1020). In some embodiments, displaying the workload user interface further comprises: in accordance with a determination that the second workload value exceeds the aggregated workload value (e.g., exceeds the aggregated workload value for the second day), displaying the second workload indication in a third manner (e.g., with a third set of visual characteristics) (e.g., at a third size, with a third color, with a third shape, within a third display region, and/or at a third display position) (e.g., displaying the second workload indication above the aggregated workload indication) (e.g., in some embodiments, a third manner this is the same as the first manner or different from the first manner); and in accordance with a determination that the second workload value does not exceed the aggregated workload value (e.g., does not exceed and/or falls below the aggregated workload value for the second day), displaying the second workload indication in a fourth manner different from the third manner (e.g., with a fourth set of visual characteristics) (e.g., at a fourth size, with a fourth color, with a fourth shape, within a fourth display region, and/or at a fourth display position) (e.g., displaying the second workload indication below the aggregated workload indication) (e.g., in some embodiments, a fourth manner that is the same as the second manner or different from the second manner). Displaying an aggregated workload indication allows the user to quickly see whether their workload for a given day is above or below their typical workload, thereby improving visual feedback to the user. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, a first instance of the workload user interface (e.g., 1016 in FIG. 10R), including concurrently displaying: the first workload indication (e.g., 1022a, 1022b, 1022c, 1022d, 1022c, 1022f, and/or 1022g in FIG. 10R) representative of the first day at a first display position that is indicative of the first workload value; the second workload indication (e.g., 1022a, 1022b, 1022c, 1022d, 1022e, 1022f, and/or 1022g in FIG. 10R) representative of the second day at a second display position that is indicative of the second workload value; and the aggregated workload indication (e.g., 1020 in FIG. 10R) at a third display position that is indicative of the first set of aggregated workload values (e.g., in some embodiments, a line, chart, and/or graph that spans a plurality of display positions based on the first set of aggregated workload values). Subsequent to displaying the first instance of the workload user interface, the computer system detects, via the one or more input devices, a first set of user inputs (e.g., one or more user inputs) (e.g., one or more touch inputs, one or more hardware inputs, one or more button presses, one or more rotational inputs, one or more gesture inputs, and/or one or more spoken inputs) corresponding to a user request to modify the workout effort value for the first workout from the first workout effort value to a first modified workout effort value that is different from the first workout effort value (e.g., 1033, 1037, 1038, and/or 1039). Subsequent to detecting the first set of user inputs, the computer system displays, via the one or more display generation components, a second instance of the workload user interface (e.g., 1016 in FIG. 10Y), including displaying the first workload indication representative of the first day at a fourth display position that is different from the first display position and that is indicative of a third workload value different from the first workload value, wherein the workload value for the first day is changed from the first workload value to the third workload value and the display position of the first workload indication is changed from the first display position to the fourth display position based on the first set of user inputs (e.g., based on the user changing the workout effort value for the first workout from the first workout effort value to the first modified workout effort value) (e.g., the positions of objects 1022e, 1022f, and 1022g change from FIG. 10R to FIG. 10Y based on modification of effort level in FIGS. 10S-10X). In some embodiments, displaying the second instance of the workload user interface includes concurrently displaying the first workload indication at the fourth display position, and the second workload indication representative of the second day at a fifth display position that is different from the second display position and that is indicative of a fourth workload value different from the second workload value, wherein the workload value for the second day is changed from the second workload value to the fourth workload value and the display position of the second workload indication is changed from the second display position to the fifth display position based on the first set of user inputs (e.g., based on the user changing the workout effort value for the first workout from the first workout effort value to the first modified workout effort value). In some embodiments, changing the workout effort value for a single workout changes the workload values for a plurality of days (e.g., a plurality of days that follow the day on which the workout was performed) due to the workload values for subsequent days being the result of an average (e.g., a weighed or non-weighted average) of workload values across a plurality of days. Allowing a user to modify the workout effort value for a workout, and then displaying updated workload information based on the user modified workout effort value, provides the user with visual feedback about a state of the system (e.g., that the system has received the modified workout effort value and has modified the workload value accordingly). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, displaying the second instance of the workload user interface (e.g., 1016 in FIG. 10Y) includes displaying, concurrently with the first workload indication at the fourth display position, the aggregated workload indication (e.g., 1020) at a fifth display position that is different from the third display position and that is indicative of a second set of aggregated workload values different from the first set of aggregated workload values, wherein: the second set of aggregated workload values differs from the first set of aggregated workload values based on the first set of user inputs (e.g., based on and/or as a result of the user changing the workout effort value for the first workout from the first workout effort value to the first modified workout effort value); and the aggregated workload indication is displayed at the fifth display position different from the third display position based on the first set of user inputs (e.g., based on the first set of user inputs changing the aggregated workload values from the first set of aggregated workload values to the second set of aggregated workload values) (e.g., line 1020 moves from FIG. 10R to FIG. 10Y based on modification of effort level in FIGS. 10S-10X). Allowing a user to modify the workout effort value for a workout, and then displaying updated workload information based on the user modified workout effort value, provides the user with visual feedback about a state of the system (e.g., that the system has received the modified workout effort value and has modified the workload value accordingly). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the workload user interface (e.g., 1016) includes a vital metrics indication (e.g., 1016c). In some embodiments, displaying the workload user interface includes: in accordance with a determination that the first day is selected within the workload user interface (e.g., in some embodiments, the first workload indication representative of the first day is selected) (e.g., and the second day is not selected), displaying, within the vital metrics indication, an indication of whether one or more vital metrics of a set of vital metrics (e.g., one or more vital metrics different from workout effort and/or workload) (e.g., heart rate, respiratory rate, body temperature, blood oxygen level, and/or sleep duration) for the user of the computer system are outside of a threshold range for the first day (e.g., in some embodiments, without displaying an indication of whether one or more vital metrics of a set of vital metrics are outside of the threshold range for the second day) (e.g., in FIG. 10Q, Monday is selected and platter 1016c indicates no outliers for Monday); and in accordance with a determination that the second day is selected within the workload user interface (e.g., in some embodiments, the second workload indication representative of the second day is selected) (e.g., and the first day is not selected), displaying, within the vital metrics indication, an indication of whether one or more vital metrics of the set of vital metrics (e.g., one or more vital metrics different from workout effort and/or workload) (e.g., heart rate, respiratory rate, body temperature, blood oxygen level, and/or sleep duration) for the user of the computer system are outside of a threshold range for the second day (e.g., in some embodiments, without displaying an indication of whether one or more vital metrics of a set of vital metrics are outside of the threshold range for the first day) (e.g., in FIG. 10R, Sunday is selected, and platter 1016c indicates two outliers for Sunday). Displaying an indication of whether one or more vital metrics are within or outside of a threshold range provides the user with visual feedback about a state of the system (e.g., that the system bas detected that the user's vital metrics are within the threshold range or outside of the threshold range). Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, while displaying the workload user interface (e.g., 1016), the computer system detects, via the one or more input devices, a selection input corresponding to selection of the vital metrics indication (e.g., one or more user inputs, one or more touch inputs, one or more tap inputs, one or more hardware control inputs, one or more button presses, one or more rotational inputs, one or more gesture inputs, and/or one or more spoken inputs) (e.g., user input selecting platter 1016c). In response to detecting the selection input corresponding to selection of the vital metrics indication, the computer system displays, via the one or more display generation components, a daily metrics user interface (e.g., 608), including displaying, within the daily metrics user interface, first vital metric information that is representative of the first day (e.g., a first day of the week, a first calendar day, and/or a first twenty-four hour period) (in some embodiments, the day user interface is representative of a first day without being representative of a second day and/or any other day) (e.g., first vital metric information that was captured during the first day and/or otherwise corresponds to the first day) (e.g., without displaying second vital metric information that is representative of a second day; without displaying vital metric information that corresponds to days other than the first day; without displaying vital metric information that does not correspond to the first day; and/or without displaying vital metric information that is not representative of the first day), wherein the first vital metric information corresponds to the set of vital metrics, and the set of vital metrics includes two or more vital metrics (e.g., heart rate, respiratory rate, body temperature, blood-oxygen level, and/or sleep duration). Providing the user with a selectable user interface object that is selectable to display a daily metrics user interface enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, within the workload user interface (e.g., 1016 and/or 1044), a first set of workload information that corresponds to a first category of physical activity (e.g., a first workout type (e.g., running workouts, swimming workouts, cycling workouts, and/or strength workouts), all workouts, and/or all day physical activity (e.g., steps taken, miles walked, and/or calories burned throughout the day)), including displaying the first workload indication that is representative of the first day at a first display position that is representative of a first workload value that is calculated for the user for the first day based on the first category of physical activity (e.g., in FIG. 10AA, user interface 1044 displays “all workouts” workload information). While displaying the first set of workload information within the workload user interface, the computer system detects, via the one or more input devices, a selection input (e.g., one or more user inputs, one or more touch inputs, one or more tap inputs, one or more hardware control inputs, one or more button presses, one or more rotational inputs, one or more gesture inputs, and/or one or more spoken inputs) corresponding to user selection of a second category of physical activity different from the first category of physical activity (e.g., 1057). In response to detecting the selection input corresponding to user selection of the second category of physical activity, the computer system displays, within the workload user interface, a second set of workload information that is different from the first set of workload information and that corresponds to the second category of physical activity, including displaying the first workload indication that is representative of the first day at a second display position that is different from the first display position and that is representative of a second workload value that is different from the first workload value, wherein the second workload value is calculated for the user for the first day based on the second category of physical activity (e.g., in FIG. 10AB, objects 1052a-1052g move from where they were in FIG. 10AA, and now display “running” workouts workload information). Providing the user with selectable options to change the workload information that is displayed within the workload user interface enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the workload user interface displays workload information that corresponds to a workload metric. In some embodiments, the computer system displays concurrently with the workload user interface (e.g., 1042) (e.g., in some embodiments, platter 1042 displays user interface 1016 and/or chart 1018), a first vital metrics user interface that corresponds to a first vital metric (e.g., heart rate, blood pressure, respiratory rate, blood oxygen level, body temperature, and/or sleep duration) that is different from the workload metric, wherein the first vital metrics user interface depicts measurements of the first vital metric over time (e.g., in some embodiments, platter 646a displays user interface 622 instead of user interface 608) (e.g., in some embodiments, platter 646b displays heart rate values charted over time; platter 646d displays calories burned values for each day charted over a period of multiple days; and/or platter 646e displays steps taken each day charted over a time period of multiple days). In some embodiments, the computer system displays, concurrently with the workload user interface and the first vital metrics user interface, a second vital metrics user interface that corresponds to a second vital metric that is different from the workload metric and the first vital metric, wherein the second vital metrics user interface depicts measurements of the second vital metric over time. Concurrently displaying multiple vital metric user interfaces allows the user to view changes in different vital metrics over time, which can help the user to identify potential patterns and/or relationships between different vital metrics. Doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays via the one or more display generation components, a time user interface (e.g., 638) (e.g., a user interface that includes an analog and/or digital indication of time, a clock face user interface, a watch face user interface, a reduced-power screen, a wake screen, and/or a lock screen), wherein: the time user interface includes a first displayed user interface object (e.g., 1070) that corresponds to the workload user interface (e.g., a first watch face complication) (e.g., in some embodiments, complication 1070 displays user interface 1016 and/or chart 1018); and the first displayed user interface object displays a representation of the workload user interface (e.g., displays the workload user interface and/or a smaller version of the workload user interface) (e.g., in some embodiments, complication 1070 displays user interface 1016 and/or chart 1018). In some embodiments, the first displayed user interface object (e.g., 1070), when selected, causes the computer system to cease display of the time user interface (e.g., 638) and display the workload user interface (e.g., 1016). Displaying the workload user interface as a watch complication allows a user to access the workload user interface with fewer user inputs. Furthermore, doing so also enbances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system (e.g., 640) displays, via the one or more display generation components, a lock screen transition user interface (e.g., 676), wherein: the lock screen transition user interface is indicative of the computer system having transitioned from a locked state to an unlocked state (e.g., an unlocked state that has access to more features than the locked state) (e.g., having transitioned from the locked state to the unlocked state in response to receiving user authentication information (e.g., passcode-based authentication and/or biometric authentication)); the lock screen transition user interface includes a second displayed user interface object (e.g., 1668) (e.g., a widget and/or complication) that corresponds to the workload user interface (e.g., 1044); and the second displayed user interface object displays a representation of the workload user interface (e.g., displays the workload user interface and/or a smaller version of the workload user interface) (e.g., in some embodiments, complication 1668 displays user interface 1044 and/or chart 1048). In some embodiments, the second displayed user interface object (e.g., 1668), when selected, causes the computer system to cease display of the lock screen transition user interface (e.g., 676) and display the workload user interface (e.g., 1044). Displaying the workload user interface as a lock screen complication allows a user to access the workload user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

In some embodiments, the computer system displays, via the one or more display generation components, an application selection user interface (e.g., 680), wherein: the application selection user interface includes: a representation of a first application (e.g., one of application icons 682a-682m) that, when selected, causes the computer system to open the first application; a representation of a second application (e.g., one of application icons 682a-682m) that, when selected, causes the computer system to open the second application; and a third displayed user interface object (e.g., 1066) (e.g., a widget and/or complication) that corresponds to the workload user interface (e.g., 1044). In some embodiments, the third displayed user interface object displays a representation of the workload user interface (e.g., displays the workload user interface and/or a smaller version of the workload user interface) (e.g., in some embodiments, widget 1066 displays user interface 1044 and/or chart 1048). In some embodiments, the third displayed user interface object (e.g., 1066), when selected, causes the computer system to cease display of the application selection user interface (e.g., 680) and display the workload user interface (e.g., 1044). Displaying the workload user interface as a lock screen complication allows a user to access the workload user interface with fewer user inputs. Furthermore, doing so also enhances the operability of the system and makes the user-system interface more efficient (e.g., by preventing erroneous inputs and helping the user to provide proper inputs and reducing errors) which, additionally, reduces power usage and improves the battery life of the device by enabling the user to use the system more quickly and efficiently.

Note that details of the processes described above with respect to method 1100 (e.g., FIG. 11) are also applicable in an analogous manner to the methods described above. For example, methods 700, 800, and/or 900 optionally include one or more of the characteristics of the various methods described above with reference to method 1100. For example, in some embodiments, the same computer system is used to perform methods 700, 800, 900, and/or 1100. In another example, in some embodiments, the alert recited in method 900 pertains to data shown in the metrics user interfaces and/or workload user interfaces recited in methods 700, 800, and/or 1100. For brevity, these details are not repeated below.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to improve the delivery to users of health data or any other content that may be of interest to them. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to deliver targeted content and/or data that is of greater interest to the user. Accordingly, use of such personal information data enables users to have calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of tracking and presenting user health metrics, the present technology can be configured to allow users to select to “opt in” or “opt out” of participation in the collection of personal information data during registration for services or anytime thereafter. In another example, users can select not to provide health data or other personal data. In yet another example, users can select to limit the length of time health data or other data is maintained or entirely prohibit the tracking of health data and/or metrics. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences based on non-personal information data or a bare minimum amount of personal information, such as the content being requested by the device associated with a user, other non-personal information available to the content delivery services, or publicly available information.

1-96. (canceled)

97. A computer system configured to communicate with one or more display generation components, comprising:
one or more processors; and
memory storing one or more programs configured to be executed by the one or more processors, the one or more programs including instructions for:
displaying, via the one or more display generation components, a metrics user interface that is representative of a first duration of time, wherein:
the metrics user interface includes a first region that is representative of a first day of the first duration of time; and
the first region includes a first set of objects that are representative of a set of vital metrics for the first day, wherein the first set of vital metrics includes two or more vital metrics; and
displaying the metrics user interface includes:
in accordance with a determination that at least one or more vital metrics of the set of vital metrics are within a threshold range for the first day, displaying a first subset of the first set of objects within a first section of the first region, wherein the first section of the first region is representative of the threshold range; and
in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

98. The computer system of claim 97, wherein displaying the metrics user interface further includes:
in accordance with a determination that none of the set of vital metrics are outside the threshold range for the first day, forgoing displaying the second subset of the first set of objects within the second section of the first region.

99. The computer system of claim 97, wherein:
displaying the first subset of the first set of objects within the first section of the first region includes displaying a first object; and
the first object is representative of a plurality of vital metrics of the set of vital metrics, including a first vital metric and a second vital metric different from the first vital metric.

100. The computer system of claim 99, wherein:
a first end of the first object is representative of the first vital metric; and
a second end of the first object different from the first end is representative of the second vital metric.

101. The computer system of claim 97, wherein the set of vital metrics includes two or more of: heart rate, respiratory rate, body temperature, blood oxygen level, and sleep duration.

102. The computer system of claim 97, wherein:
the metrics user interface includes a second region that is separate from the first region and is representative of a second day of the first duration of time different from the first day;
the second region includes a second set of objects that are separate from the first set of objects and are representative of the set of vital metrics for the second day;
and displaying the metrics user interface includes:
in accordance with a determination that at least one or more vital metrics of the set of vital metrics are within the threshold range for the second day, displaying a first subset of the second set of objects within a first section of the second region, wherein the first section of the second region is representative of the threshold range; and
in accordance with a determination that at least one or more vital metrics of the set of vital metrics are outside the threshold range for the second day, displaying a second subset of the second set of objects within a second section of the second region, wherein the second section of the second region is different from the first section of the second region and is indicative of being outside of the threshold range.

103. The computer system of claim 102, wherein displaying the metrics user interface further includes:
in accordance with a determination that at least one or more vital metrics of the set of vital metrics are outside of the threshold range for the first day, displaying the first set of objects in a first color; and
in accordance with a determination that none of the vital metrics of the set of vital metrics is outside of the threshold range for the first day, displaying the first set of objects in a second color different from the first color.

104. The computer system of claim 102, the one or more programs further including instructions for:
while displaying the metrics user interface in a first manner indicating that the first day is selected and the second day is not selected, detecting rotation of a rotatable input mechanism; and
in response to detecting rotation of the rotatable input mechanism, displaying the metrics user interface in a second manner indicating that the second day is selected and the first day is not selected.

105. The computer system of claim 104, the one or more programs further including instructions for:
while the first day is selected and while displaying the metrics user interface in the first manner, displaying, within the metrics user interface, a textual indication of a number of vital metrics of the set of vital metrics that are outside the threshold range for the first day without displaying a textual indication of a number of vital metrics of the set of vital metrics that are outside the threshold range for the second day; and
while the second day is selected and while displaying the metrics user interface in the second manner, displaying, within the metrics user interface, the textual indication of the number of vital metrics of the set of vital metrics that are outside the threshold range for the second day without displaying the textual indication of the number of vital metrics of the set of vital metrics that are outside the threshold range for the first day.

106. The computer system of claim 102, the one or more programs further including instructions for:
while displaying the metrics user interface, detecting, via one or more input devices, a first user input corresponding to a user request to display a second metrics user interface different from the metrics user interface; and
in response to detecting the first user input:
in accordance with a determination that the first day was selected when the first user input was received, displaying, via the one or more display generation components, a first respective metrics user interface, wherein the first respective metrics user interface displays first vital metric information that is representative of the first day and the set of vital metrics for the first day without displaying the set of vital metrics for the second day; and
in accordance with a determination that the second day was selected when the first user input was received, displaying, via the one or more display generation components, a second respective metrics user interface, wherein the second respective metrics user interface displays second vital metric information that is representative of the second day and the set of vital metrics for the second day without displaying the set of vital metrics for the first day.

107. The computer system of claim 102, wherein:
the metrics user interface includes a third region that is separate from the first region and the second region and is representative of a third day of the first duration of time different from the first day and the second day;
the third region includes a third set of objects that are separate from the first set of objects and the second set of objects and are representative of the set of vital metrics for the third day;
and displaying the metrics user interface includes:
in accordance with a determination that at least one or more vital metrics of the set of vital metrics are within the threshold range for the third day, displaying a first subset of the third set of objects within a first section of the third region, wherein the first section of the third region is representative of the threshold range; and
in accordance with a determination that at least one or more vital metrics of the set of vital metrics are outside the threshold range for the third day, displaying a second subset of the third set of objects within a second section of the third region, wherein the second section of the third region is different from the first section of the third region and is indicative of being outside of the threshold range.

108. The computer system of claim 97, the one or more programs further including instructions for:
displaying, via the one or more display generation components, a time user interface, wherein:
the time user interface includes a first displayed user interface object that corresponds to the metrics user interface; and
the first displayed user interface object displays a representation of the metrics user interface.

109. The computer system of claim 97, the one or more programs further including instructions for:
displaying, via the one or more display generation components, a lock screen transition user interface, wherein:
the lock screen transition user interface is indicative of the computer system having transitioned from a locked state to an unlocked state;
the lock screen transition user interface includes a second displayed user interface object that corresponds to the metrics user interface; and
the second displayed user interface object displays a representation of the metrics user interface.

110. The computer system of claim 97, the one or more programs further including instructions for:
displaying, via the one or more display generation components, an application selection user interface, wherein:
the application selection user interface includes:
a representation of a first application that, when selected, causes the computer system to open the first application;
a representation of a second application that, when selected, causes the computer system to open the second application; and
a third displayed user interface object that corresponds to the metrics user interface; and
the third displayed user interface object displays a representation of the metrics user interface.

111. A non-transitory computer-readable storage medium storing one or more programs configured to be executed by one or more processors of a computer system that is in communication with one or more display generation components, the one or more programs including instructions for:
displaying, via the one or more display generation components, a metrics user interface that is representative of a first duration of time, wherein:
the metrics user interface includes a first region that is representative of a first day of the first duration of time; and
the first region includes a first set of objects that are representative of a set of vital metrics for the first day, wherein the first set of vital metrics includes two or more vital metrics; and
displaying the metrics user interface includes:
in accordance with a determination that at least one or more vital metrics of the set of vital metrics are within a threshold range for the first day, displaying a first subset of the first set of objects within a first section of the first region, wherein the first section of the first region is representative of the threshold range; and
in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.

112. A method, comprising:
at a computer system that is in communication with one or more display generation components:
displaying, via the one or more display generation components, a metrics user interface that is representative of a first duration of time, wherein:
the metrics user interface includes a first region that is representative of a first day of the first duration of time; and
the first region includes a first set of objects that are representative of a set of vital metrics for the first day, wherein the first set of vital metrics includes two or more vital metrics; and
displaying the metrics user interface includes:
in accordance with a determination that at least one or more vital metrics of the set of vital metrics are within a threshold range for the first day, displaying a first subset of the first set of objects within a first section of the first region, wherein the first section of the first region is representative of the threshold range; and
in accordance with a determination that at least one or more vital metrics of the set of vital metrics are outside the threshold range for the first day, displaying a second subset of the first set of objects within a second section of the first region, wherein the second section of the first region is different from the first section of the first region and is indicative of being outside of the threshold range.
